SEQUENCE LISTING (1046 sequences)

SEQ ID NO: 1 - protein, 8 aa, Artificial Sequence
  Artificial peptide sequence
  MISC_FEATURE (1)..(1): Xaa in position 1 is A, L, I, or V
  MISC_FEATURE (2)..(2): Xaa in position 2 is S, T, G, A, N, E or D
  MISC_FEATURE (3)..(3): Xaa in position 3 is D, E, or G
  MISC_FEATURE (5)..(5): Xaa in position 5 is P, N, S, D, A, T, K, or G
  MISC_FEATURE (6)..(6): Xaa in position 6 is Y, N, I, W, or F
  MISC_FEATURE (7)..(7): Xaa in position 7 is I, L, R, or V
  MISC_FEATURE (8)..(8): Xaa in position 8 is L, I, V, S, M, or T
XXXKXXXX

SEQ ID NO: 2 - protein, 8 aa, Artificial Sequence
  Artificial peptide sequence
  MISC_FEATURE (1)..(1): Xaa in position 1 is N-term (-NH2) A, L, I, or V
  MISC_FEATURE (2)..(2): Xaa in position 2 is S, T, G, A, N, E or D
  MISC_FEATURE (3)..(3): Xaa in position 3 is D, E, or G
  MISC_FEATURE (5)..(5): Xaa in position 5 is P, N, S, D, A, T, K, or G
  MISC_FEATURE (6)..(6): Xaa in position 6 is Y, N, I, W, or F
  MISC_FEATURE (7)..(7): Xaa in position 7 is I, L, R, or V
  MISC_FEATURE (8)..(8): Xaa in position 8 is a C-term L, I, V, S, M, or T
XXXKXXXX

SEQ ID NO: 3 - protein, 8 aa, Artificial Sequence
  Artificial peptide sequence
  MISC_FEATURE (1)..(1): Xaa in position 1 is A, L, I, or V
  MISC_FEATURE (2)..(2): Xaa in position 2 is S, T, G, A, N, E or D
  MISC_FEATURE (3)..(3): Xaa in position 3 is D, R, K, E, or G
  MISC_FEATURE (4)..(4): Xaa in position 4 is K or R
  MISC_FEATURE (5)..(5): Xaa in position 5 is P, N, S, D, A, T, K, or G
  MISC_FEATURE (6)..(6): Xaa in position 6 is Y, N, I, W, or F
  MISC_FEATURE (7)..(7): Xaa in position 7 is I, L, R, or V
  MISC_FEATURE (8)..(8): Xaa in position 8 is L, I, V, S, M, or T
XXXXXXXX

SEQ ID NO: 4 - protein, 4 aa, Artificial Sequence
  Artificial peptide sequence
PYIL

SEQ ID NO: 5 - protein, 16 aa, Artificial Sequence
  Artificial peptide sequence
AVTEKKYILYDFSVTS

SEQ ID NO: 6 - protein, 8 aa, Artificial Sequence
  Artificial peptide sequence
ASDKPYIL

SEQ ID NO: 7 - protein, 7 aa, Artificial Sequence
  Artificial peptide sequence
SDKPYIL

SEQ ID NO: 8 - protein, 6 aa, Artificial Sequence
  Artificial peptide sequence
DKPYIL

SEQ ID NO: 9 - protein, 5 aa, Artificial Sequence
  Artificial peptide sequence
KPYIL

SEQ ID NO: 10 - protein, 8 aa, Artificial Sequence
  Artificial peptide sequence
AGDKNYIL

SEQ ID NO: 11 - protein, 8 aa, Artificial Sequence
  Artificial peptide sequence
AGDKNYIT

SEQ ID NO: 12 - protein, 8 aa, Artificial Sequence
  Artificial peptide sequence
AGDKSYIT

SEQ ID NO: 13 - protein, 8 aa, Artificial Sequence
  Artificial peptide sequence
ADGKPYIV

SEQ ID NO: 14 - protein, 8 aa, Artificial Sequence
  Artificial peptide sequence
AEDKDFIT

SEQ ID NO: 15 - protein, 8 aa, Artificial Sequence
  Artificial peptide sequence
AADKPYIL

SEQ ID NO: 16 - protein, 8 aa, Artificial Sequence
  Artificial peptide sequence
ATDKPYIL

SEQ ID NO: 17 - protein, 8 aa, Artificial Sequence
  Artificial peptide sequence
AGDKPYIT

SEQ ID NO: 18 - protein, 8 aa, Artificial Sequence
  Artificial peptide sequence
ASEKPYIL

SEQ ID NO: 19 - protein, 8 aa, Artificial Sequence
  Artificial peptide sequence
ADGKPYVT

SEQ ID NO: 20 - protein, 8 aa, Artificial Sequence
  Artificial peptide sequence
AGDKPYIL

SEQ ID NO: 21 - protein, 8 aa, Artificial Sequence
  Artificial peptide sequence
ASDKPNIL

SEQ ID NO: 22 - protein, 8 aa, Artificial Sequence
  Artificial peptide sequence
ASDKPYIT

SEQ ID NO: 23 - protein, 8 aa, Artificial Sequence
  Artificial peptide sequence
AADKPFIL

SEQ ID NO: 24 - protein, 8 aa, Artificial Sequence
  Artificial peptide sequence
ASDKAYIT

SEQ ID NO: 25 - protein, 8 aa, Artificial Sequence
  Artificial peptide sequence
AGDKAYIT

SEQ ID NO: 26 - protein, 8 aa, Artificial Sequence
  Artificial peptide sequence
ANGKPFIT

SEQ ID NO: 27 - protein, 8 aa, Artificial Sequence
  Artificial peptide sequence
AGDKNFIT

SEQ ID NO: 28 - protein, 8 aa, Artificial Sequence
  Artificial peptide sequence
ASDKSYIT

SEQ ID NO: 29 - protein, 8 aa, Artificial Sequence
  Artificial peptide sequence
ASDKTYIT

SEQ ID NO: 30 - protein, 8 aa, Artificial Sequence
  Artificial peptide sequence
ASDKNYIT

SEQ ID NO: 31 - protein, 8 aa, Artificial Sequence
  Artificial peptide sequence
AGDKKYIT

SEQ ID NO: 32 - protein, 8 aa, Artificial Sequence
  Artificial peptide sequence
AGDKNYIS

SEQ ID NO: 33 - protein, 8 aa, Artificial Sequence
  Artificial peptide sequence
AADKNYIT

SEQ ID NO: 34 - protein, 8 aa, Artificial Sequence
  Artificial peptide sequence
AGDKNYIM

SEQ ID NO: 35 - protein, 8 aa, Artificial Sequence
  Artificial peptide sequence
AADKNFIM

SEQ ID NO: 36 - protein, 8 aa, Artificial Sequence
  Artificial peptide sequence
AADKNFIT

SEQ ID NO: 37 - protein, 8 aa, Artificial Sequence
  Artificial peptide sequence
AGDKGIRS

SEQ ID NO: 38 - protein, 7 aa, Artificial Sequence
  Artificial peptide sequence
PRRPYIL

SEQ ID NO: 39 - protein, 6 aa, Artificial Sequence
  Artificial peptide sequence
RRPYIL

SEQ ID NO: 40 - protein, 5 aa, Artificial Sequence
  Artificial peptide sequence
RPYIL

SEQ ID NO: 41 - protein, 6 aa, Artificial Sequence
  Artificial peptide sequence
RRPWIL

SEQ ID NO: 42 - protein, 6 aa, Artificial Sequence
  Artificial peptide sequence
KRPYIL

SEQ ID NO: 43 - protein, 6 aa, Artificial Sequence
  Artificial peptide sequence
KKPYIL

SEQ ID NO: 44 - protein, 6 aa, Artificial Sequence
  Artificial peptide sequence
  misc_feature (2)..(2): Xaa can be any naturally occurring amino acid
KXPYIL

SEQ ID NO: 45 - protein, 5 aa, Artificial Sequence
  Artificial peptide sequence
KPYII

SEQ ID NO: 46 - protein, 5 aa, Artificial Sequence
  Artificial peptide sequence
KPYIV

SEQ ID NO: 47 - protein, 5 aa, Artificial Sequence
  Artificial peptide sequence
KPYLL

SEQ ID NO: 48 - protein, 5 aa, Artificial Sequence
  Artificial peptide sequence
KPYLI

SEQ ID NO: 49 - protein, 5 aa, Artificial Sequence
  Artificial peptide sequence
KPYLV

SEQ ID NO: 50 - protein, 5 aa, Artificial Sequence
  Artificial peptide sequence
KPYVL

SEQ ID NO: 51 - protein, 5 aa, Artificial Sequence
  Artificial peptide sequence
KPYVI

SEQ ID NO: 52 - protein, 5 aa, Artificial Sequence
  Artificial peptide sequence
KPYVV

SEQ ID NO: 53 - protein, 5 aa, Artificial Sequence
  Artificial peptide sequence
KPWIL

SEQ ID NO: 54 - protein, 5 aa, Artificial Sequence
  Artificial peptide sequence
KPWII

SEQ ID NO: 55 - protein, 5 aa, Artificial Sequence
  Artificial peptide sequence
KPWIV

SEQ ID NO: 56 - protein, 5 aa, Artificial Sequence
  Artificial peptide sequence
KPWLL

SEQ ID NO: 57 - protein, 5 aa, Artificial Sequence
  Artificial peptide sequence
KPWLI

SEQ ID NO: 58 - protein, 5 aa, Artificial Sequence
  Artificial peptide sequence
KPWLV

SEQ ID NO: 59 - protein, 5 aa, Artificial Sequence
  Artificial peptide sequence
KPWVL

SEQ ID NO: 60 - protein, 5 aa, Artificial Sequence
  Artificial peptide sequence
KPWVI

SEQ ID NO: 61 - protein, 5 aa, Artificial Sequence
  Artificial peptide sequence
KPWVV

SEQ ID NO: 62 - protein, 5 aa, Artificial Sequence
  Artificial peptide sequence
RPYII

SEQ ID NO: 63 - protein, 5 aa, Artificial Sequence
  Artificial peptide sequence
RPYIV

SEQ ID NO: 64 - protein, 5 aa, Artificial Sequence
  Artificial peptide sequence
RPYLL

SEQ ID NO: 65 - protein, 5 aa, Artificial Sequence
  Artificial peptide sequence
RPYLI

SEQ ID NO: 66 - protein, 5 aa, Artificial Sequence
  Artificial peptide sequence
RPYLV

SEQ ID NO: 67 - protein, 5 aa, Artificial Sequence
  Artificial peptide sequence
RPYVL

SEQ ID NO: 68 - protein, 5 aa, Artificial Sequence
  Artificial peptide sequence
RPYVI

SEQ ID NO: 69 - protein, 5 aa, Artificial Sequence
  Artificial peptide sequence
RPYVV

SEQ ID NO: 70 - protein, 5 aa, Artificial Sequence
  Artificial peptide sequence
RPWIL

SEQ ID NO: 71 - protein, 5 aa, Artificial Sequence
  Artificial peptide sequence
RPWII

SEQ ID NO: 72 - protein, 5 aa, Artificial Sequence
  Artificial peptide sequence
RPWIV

SEQ ID NO: 73 - protein, 5 aa, Artificial Sequence
  Artificial peptide sequence
RPWLL

SEQ ID NO: 74 - protein, 5 aa, Artificial Sequence
  Artificial peptide sequence
RPWLI

SEQ ID NO: 75 - protein, 5 aa, Artificial Sequence
  Artificial peptide sequence
RPWLV

SEQ ID NO: 76 - protein, 5 aa, Artificial Sequence
  Artificial peptide sequence
RPWVL

SEQ ID NO: 77 - protein, 5 aa, Artificial Sequence
  Artificial peptide sequence
RPWVI

SEQ ID NO: 78 - protein, 5 aa, Artificial Sequence
  Artificial peptide sequence
RPWVV

SEQ ID NO: 79 - protein, 6 aa, Artificial Sequence
  Artificial peptide sequence
DKPYII

SEQ ID NO: 80 - protein, 6 aa, Artificial Sequence
  Artificial peptide sequence
DKPYIV

SEQ ID NO: 81 - protein, 6 aa, Artificial Sequence
  Artificial peptide sequence
DKPYLL

SEQ ID NO: 82 - protein, 6 aa, Artificial Sequence
  Artificial peptide sequence
DKPYLI

SEQ ID NO: 83 - protein, 6 aa, Artificial Sequence
  Artificial peptide sequence
DKPYLV

SEQ ID NO: 84 - protein, 6 aa, Artificial Sequence
  Artificial peptide sequence
DKPYVL

SEQ ID NO: 85 - protein, 6 aa, Artificial Sequence
  Artificial peptide sequence
DKPYVI

SEQ ID NO: 86 - protein, 6 aa, Artificial Sequence
  Artificial peptide sequence
DKPYVV

SEQ ID NO: 87 - protein, 6 aa, Artificial Sequence
  Artificial peptide sequence
DKPWIL

SEQ ID NO: 88 - protein, 6 aa, Artificial Sequence
  Artificial peptide sequence
DKPWII

SEQ ID NO: 89 - protein, 6 aa, Artificial Sequence
  Artificial peptide sequence
DKPWIV

SEQ ID NO: 90 - protein, 6 aa, Artificial Sequence
  Artificial peptide sequence
DKPWLL

SEQ ID NO: 91 - protein, 6 aa, Artificial Sequence
  Artificial peptide sequence
DKPWLI

SEQ ID NO: 92 - protein, 6 aa, Artificial Sequence
  Artificial peptide sequence
DKPWLV

SEQ ID NO: 93 - protein, 6 aa, Artificial Sequence
  Artificial peptide sequence
DKPWVL

SEQ ID NO: 94 - protein, 6 aa, Artificial Sequence
  Artificial peptide sequence
DKPWVI

SEQ ID NO: 95 - protein, 6 aa, Artificial Sequence
  Artificial peptide sequence
DKPWVV

SEQ ID NO: 96 - protein, 6 aa, Artificial Sequence
  Artificial peptide sequence
DRPYIL

SEQ ID NO: 97 - protein, 6 aa, Artificial Sequence
  Artificial peptide sequence
DRPYII

SEQ ID NO: 98 - protein, 6 aa, Artificial Sequence
  Artificial peptide sequence
DRPYIV

SEQ ID NO: 99 - protein, 6 aa, Artificial Sequence
  Artificial peptide sequence
DRPYLL

SEQ ID NO: 100 - protein, 6 aa, Artificial Sequence
  Artificial peptide sequence
DRPYLI

SEQ ID NO: 101 - protein, 6 aa, Artificial Sequence
  Artificial peptide sequence
DRPYLV

SEQ ID NO: 102 - protein, 6 aa, Artificial Sequence
  Artificial peptide sequence
DRPYVL

SEQ ID NO: 103 - protein, 6 aa, Artificial Sequence
  Artificial peptide sequence
DRPYVI

SEQ ID NO: 104 - protein, 6 aa, Artificial Sequence
  Artificial peptide sequence
DRPYVV

SEQ ID NO: 105 - protein, 6 aa, Artificial Sequence
  Artificial peptide sequence
DRPWIL

SEQ ID NO: 106 - protein, 6 aa, Artificial Sequence
  Artificial peptide sequence
DRPWII

SEQ ID NO: 107 - protein, 6 aa, Artificial Sequence
  Artificial peptide sequence
DRPWIV

SEQ ID NO: 108 - protein, 6 aa, Artificial Sequence
  Artificial peptide sequence
DRPWLL

SEQ ID NO: 109 - protein, 6 aa, Artificial Sequence
  Artificial peptide sequence
DRPWLI

SEQ ID NO: 110 - protein, 6 aa, Artificial Sequence
  Artificial peptide sequence
DRPWLV

SEQ ID NO: 111 - protein, 6 aa, Artificial Sequence
  Artificial peptide sequence
DRPWVL

SEQ ID NO: 112 - protein, 6 aa, Artificial Sequence
  Artificial peptide sequence
DRPWVI

SEQ ID NO: 113 - protein, 6 aa, Artificial Sequence
  Artificial peptide sequence
DRPWVV

SEQ ID NO: 114 - protein, 6 aa, Artificial Sequence
  Artificial peptide sequence
EKPYIL

SEQ ID NO: 115 - protein, 6 aa, Artificial Sequence
  Artificial peptide sequence
EKPYII

SEQ ID NO: 116 - protein, 6 aa, Artificial Sequence
  Artificial peptide sequence
EKPYIV

SEQ ID NO: 117 - protein, 6 aa, Artificial Sequence
  Artificial peptide sequence
EKPYLL

SEQ ID NO: 118 - protein, 6 aa, Artificial Sequence
  Artificial peptide sequence
EKPYLI

SEQ ID NO: 119 - protein, 6 aa, Artificial Sequence
  Artificial peptide sequence
EKPYLV

SEQ ID NO: 120 - protein, 6 aa, Artificial Sequence
  Artificial peptide sequence
EKPYVL

SEQ ID NO: 121 - protein, 6 aa, Artificial Sequence
  Artificial peptide sequence
EKPYVI

SEQ ID NO: 122 - protein, 6 aa, Artificial Sequence
  Artificial peptide sequence
EKPYVV

SEQ ID NO: 123 - protein, 6 aa, Artificial Sequence
  Artificial peptide sequence
EKPWIL

SEQ ID NO: 124 - protein, 6 aa, Artificial Sequence
  Artificial peptide sequence
EKPWII

SEQ ID NO: 125 - protein, 6 aa, Artificial Sequence
  Artificial peptide sequence
EKPWIV

SEQ ID NO: 126 - protein, 6 aa, Artificial Sequence
  Artificial peptide sequence
EKPWLL

SEQ ID NO: 127 - protein, 6 aa, Artificial Sequence
  Artificial peptide sequence
EKPWLI

SEQ ID NO: 128 - protein, 6 aa, Artificial Sequence
  Artificial peptide sequence
EKPWLV

SEQ ID NO: 129 - protein, 6 aa, Artificial Sequence
  Artificial peptide sequence
EKPWVL

SEQ ID NO: 130 - protein, 6 aa, Artificial Sequence
  Artificial peptide sequence
EKPWVI

SEQ ID NO: 131 - protein, 6 aa, Artificial Sequence
  Artificial peptide sequence
EKPWVV

SEQ ID NO: 132 - protein, 6 aa, Artificial Sequence
  Artificial peptide sequence
ERPYIL

SEQ ID NO: 133 - protein, 6 aa, Artificial Sequence
  Artificial peptide sequence
ERPYII

SEQ ID NO: 134 - protein, 6 aa, Artificial Sequence
  Artificial peptide sequence
ERPYIV

SEQ ID NO: 135 - protein, 6 aa, Artificial Sequence
  Artificial peptide sequence
ERPYLL

SEQ ID NO: 136 - protein, 6 aa, Artificial Sequence
  Artificial peptide sequence
ERPYLI

SEQ ID NO: 137 - protein, 6 aa, Artificial Sequence
  Artificial peptide sequence
ERPYLV

SEQ ID NO: 138 - protein, 6 aa, Artificial Sequence
  Artificial peptide sequence
ERPYVL

SEQ ID NO: 139 - protein, 6 aa, Artificial Sequence
  Artificial peptide sequence
ERPYVI

SEQ ID NO: 140 - protein, 6 aa, Artificial Sequence
  Artificial peptide sequence
ERPYVV

SEQ ID NO: 141 - protein, 6 aa, Artificial Sequence
  Artificial peptide sequence
ERPWIL

SEQ ID NO: 142 - protein, 6 aa, Artificial Sequence
  Artificial peptide sequence
ERPWII

SEQ ID NO: 143 - protein, 6 aa, Artificial Sequence
  Artificial peptide sequence
ERPWIV

SEQ ID NO: 144 - protein, 6 aa, Artificial Sequence
  Artificial peptide sequence
ERPWLL

SEQ ID NO: 145 - protein, 6 aa, Artificial Sequence
  Artificial peptide sequence
ERPWLI

SEQ ID NO: 146 - protein, 6 aa, Artificial Sequence
  Artificial peptide sequence
ERPWLV

SEQ ID NO: 147 - protein, 6 aa, Artificial Sequence
  Artificial peptide sequence
ERPWVL

SEQ ID NO: 148 - protein, 6 aa, Artificial Sequence
  Artificial peptide sequence
ERPWVI

SEQ ID NO: 149 - protein, 6 aa, Artificial Sequence
  Artificial peptide sequence
ERPWVV

SEQ ID NO: 150 - protein, 6 aa, Artificial Sequence
  Artificial peptide sequence
RKPYIL

SEQ ID NO: 151 - protein, 6 aa, Artificial Sequence
  Artificial peptide sequence
RKPYII

SEQ ID NO: 152 - protein, 6 aa, Artificial Sequence
  Artificial peptide sequence
RKPYIV

SEQ ID NO: 153 - protein, 6 aa, Artificial Sequence
  Artificial peptide sequence
RKPYLL

SEQ ID NO: 154 - protein, 6 aa, Artificial Sequence
  Artificial peptide sequence
RKPYLI

SEQ ID NO: 155 - protein, 6 aa, Artificial Sequence
  Artificial peptide sequence
RKPYLV

SEQ ID NO: 156 - protein, 6 aa, Artificial Sequence
  Artificial peptide sequence
RKPYVL

SEQ ID NO: 157 - protein, 6 aa, Artificial Sequence
  Artificial peptide sequence
RKPYVI

SEQ ID NO: 158 - protein, 6 aa, Artificial Sequence
  Artificial peptide sequence
RKPYVV

SEQ ID NO: 159 - protein, 6 aa, Artificial Sequence
  Artificial peptide sequence
RKPWIL

SEQ ID NO: 160 - protein, 6 aa, Artificial Sequence
  Artificial peptide sequence
RKPWII

SEQ ID NO: 161 - protein, 6 aa, Artificial Sequence
  Artificial peptide sequence
RKPWIV

SEQ ID NO: 162 - protein, 6 aa, Artificial Sequence
  Artificial peptide sequence
RKPWLL

SEQ ID NO: 163 - protein, 6 aa, Artificial Sequence
  Artificial peptide sequence
RKPWLI

SEQ ID NO: 164 - protein, 6 aa, Artificial Sequence
  Artificial peptide sequence
RKPWLV

SEQ ID NO: 165 - protein, 6 aa, Artificial Sequence
  Artificial peptide sequence
RKPWVL

SEQ ID NO: 166 - protein, 6 aa, Artificial Sequence
  Artificial peptide sequence
RKPWVI

SEQ ID NO: 167 - protein, 6 aa, Artificial Sequence
  Artificial peptide sequence
RKPWVV

SEQ ID NO: 168 - protein, 6 aa, Artificial Sequence
  Artificial peptide sequence
RRPYII

SEQ ID NO: 169 - protein, 6 aa, Artificial Sequence
  Artificial peptide sequence
RRPYIV

SEQ ID NO: 170 - protein, 6 aa, Artificial Sequence
  Artificial peptide sequence
RRPYLL

SEQ ID NO: 171 - protein, 6 aa, Artificial Sequence
  Artificial peptide sequence
RRPYLI

SEQ ID NO: 172 - protein, 6 aa, Artificial Sequence
  Artificial peptide sequence
RRPYLV

SEQ ID NO: 173 - protein, 6 aa, Artificial Sequence
  Artificial peptide sequence
RRPYVL

SEQ ID NO: 174 - protein, 6 aa, Artificial Sequence
  Artificial peptide sequence
RRPYVI

SEQ ID NO: 175 - protein, 6 aa, Artificial Sequence
  Artificial peptide sequence
RRPYVV

SEQ ID NO: 176 - protein, 6 aa, Artificial Sequence
  Artificial peptide sequence
RRPWII

SEQ ID NO: 177 - protein, 6 aa, Artificial Sequence
  Artificial peptide sequence
RRPWIV

SEQ ID NO: 178 - protein, 6 aa, Artificial Sequence
  Artificial peptide sequence
RRPWLL

SEQ ID NO: 179 - protein, 6 aa, Artificial Sequence
  Artificial peptide sequence
RRPWLI

SEQ ID NO: 180 - protein, 6 aa, Artificial Sequence
  Artificial peptide sequence
RRPWLV

SEQ ID NO: 181 - protein, 6 aa, Artificial Sequence
  Artificial peptide sequence
RRPWVL

SEQ ID NO: 182 - protein, 6 aa, Artificial Sequence
  Artificial peptide sequence
RRPWVI

SEQ ID NO: 183 - protein, 6 aa, Artificial Sequence
  Artificial peptide sequence
RRPWVV

SEQ ID NO: 184 - protein, 6 aa, Artificial Sequence
  Artificial peptide sequence
GKPYIL

SEQ ID NO: 185 - protein, 6 aa, Artificial Sequence
  Artificial peptide sequence
GKPYII

SEQ ID NO: 186 - protein, 6 aa, Artificial Sequence
  Artificial peptide sequence
GKPYIV

SEQ ID NO: 187 - protein, 6 aa, Artificial Sequence
  Artificial peptide sequence
GKPYLL

SEQ ID NO: 188 - protein, 6 aa, Artificial Sequence
  Artificial peptide sequence
GKPYLI

SEQ ID NO: 189 - protein, 6 aa, Artificial Sequence
  Artificial peptide sequence
GKPYLV

SEQ ID NO: 190 - protein, 6 aa, Artificial Sequence
  Artificial peptide sequence
GKPYVL

SEQ ID NO: 191 - protein, 6 aa, Artificial Sequence
  Artificial peptide sequence
GKPYVI

SEQ ID NO: 192 - protein, 6 aa, Artificial Sequence
  Artificial peptide sequence
GKPYVV

SEQ ID NO: 193 - protein, 6 aa, Artificial Sequence
  Artificial peptide sequence
GKPWIL

SEQ ID NO: 194 - protein, 6 aa, Artificial Sequence
  Artificial peptide sequence
GKPWII

SEQ ID NO: 195 - protein, 6 aa, Artificial Sequence
  Artificial peptide sequence
GKPWIV

SEQ ID NO: 196 - protein, 6 aa, Artificial Sequence
  Artificial peptide sequence
GKPWLL

SEQ ID NO: 197 - protein, 6 aa, Artificial Sequence
  Artificial peptide sequence
GKPWLI

SEQ ID NO: 198 - protein, 6 aa, Artificial Sequence
  Artificial peptide sequence
GKPWLV

SEQ ID NO: 199 - protein, 6 aa, Artificial Sequence
  Artificial peptide sequence
GKPWVL

SEQ ID NO: 200 - protein, 6 aa, Artificial Sequence
  Artificial peptide sequence
GKPWVI

SEQ ID NO: 201 - protein, 6 aa, Artificial Sequence
  Artificial peptide sequence
GKPWVV

SEQ ID NO: 202 - protein, 6 aa, Artificial Sequence
  Artificial peptide sequence
GRPYIL

SEQ ID NO: 203 - protein, 6 aa, Artificial Sequence
  Artificial peptide sequence
GRPYII

SEQ ID NO: 204 - protein, 6 aa, Artificial Sequence
  Artificial peptide sequence
GRPYIV

SEQ ID NO: 205 - protein, 6 aa, Artificial Sequence
  Artificial peptide sequence
GRPYLL

SEQ ID NO: 206 - protein, 6 aa, Artificial Sequence
  Artificial peptide sequence
GRPYLI

SEQ ID NO: 207 - protein, 6 aa, Artificial Sequence
  Artificial peptide sequence
GRPYLV

SEQ ID NO: 208 - protein, 6 aa, Artificial Sequence
  Artificial peptide sequence
GRPYVL

SEQ ID NO: 209 - protein, 6 aa, Artificial Sequence
  Artificial peptide sequence
GRPYVI

SEQ ID NO: 210 - protein, 6 aa, Artificial Sequence
  Artificial peptide sequence
GRPYVV

SEQ ID NO: 211 - protein, 6 aa, Artificial Sequence
  Artificial peptide sequence
GRPWIL

SEQ ID NO: 212 - protein, 6 aa, Artificial Sequence
  Artificial peptide sequence
GRPWII

SEQ ID NO: 213 - protein, 6 aa, Artificial Sequence
  Artificial peptide sequence
GRPWIV

SEQ ID NO: 214 - protein, 6 aa, Artificial Sequence
  Artificial peptide sequence
GRPWLL

SEQ ID NO: 215 - protein, 6 aa, Artificial Sequence
  Artificial peptide sequence
GRPWLI

SEQ ID NO: 216 - protein, 6 aa, Artificial Sequence
  Artificial peptide sequence
GRPWLV

SEQ ID NO: 217 - protein, 6 aa, Artificial Sequence
  Artificial peptide sequence
GRPWVL

SEQ ID NO: 218 - protein, 6 aa, Artificial Sequence
  Artificial peptide sequence
GRPWVI

SEQ ID NO: 219 - protein, 6 aa, Artificial Sequence
  Artificial peptide sequence
GRPWVV

SEQ ID NO: 220 - protein, 7 aa, Artificial Sequence
  Artificial peptide sequence
SDKPYIL

SEQ ID NO: 221 - protein, 7 aa, Artificial Sequence
  Artificial peptide sequence
SDKPYII

SEQ ID NO: 222 - protein, 7 aa, Artificial Sequence
  Artificial peptide sequence
SDKPYIV

SEQ ID NO: 223 - protein, 7 aa, Artificial Sequence
  Artificial peptide sequence
SDKPYLL

SEQ ID NO: 224 - protein, 7 aa, Artificial Sequence
  Artificial peptide sequence
SDKPYLI

SEQ ID NO: 225 - protein, 7 aa, Artificial Sequence
  Artificial peptide sequence
SDKPYLV

SEQ ID NO: 226 - protein, 7 aa, Artificial Sequence
  Artificial peptide sequence
SDKPYVL

SEQ ID NO: 227 - protein, 7 aa, Artificial Sequence
  Artificial peptide sequence
SDKPYVI

SEQ ID NO: 228 - protein, 7 aa, Artificial Sequence
  Artificial peptide sequence
SDKPYVV

SEQ ID NO: 229 - protein, 7 aa, Artificial Sequence
  Artificial peptide sequence
SDKPWIL

SEQ ID NO: 230 - protein, 7 aa, Artificial Sequence
  Artificial peptide sequence
SDKPWII

SEQ ID NO: 231 - protein, 7 aa, Artificial Sequence
  Artificial peptide sequence
SDKPWIV

SEQ ID NO: 232 - protein, 7 aa, Artificial Sequence
  Artificial peptide sequence
SDKPWLL

SEQ ID NO: 233 - protein, 7 aa, Artificial Sequence
  Artificial peptide sequence
SDKPWLI

SEQ ID NO: 234 - protein, 7 aa, Artificial Sequence
  Artificial peptide sequence
SDKPWLV

SEQ ID NO: 235 - protein, 7 aa, Artificial Sequence
  Artificial peptide sequence
SDKPWVL

SEQ ID NO: 236 - protein, 7 aa, Artificial Sequence
  Artificial peptide sequence
SDKPWVI

SEQ ID NO: 237 - protein, 7 aa, Artificial Sequence
  Artificial peptide sequence
SDKPWVV

SEQ ID NO: 238 - protein, 7 aa, Artificial Sequence
  Artificial peptide sequence
SDRPYIL

SEQ ID NO: 239 - protein, 7 aa, Artificial Sequence
  Artificial peptide sequence
SDRPYII

SEQ ID NO: 240 - protein, 7 aa, Artificial Sequence
  Artificial peptide sequence
SDRPYIV

SEQ ID NO: 241 - protein, 7 aa, Artificial Sequence
  Artificial peptide sequence
SDRPYLL

SEQ ID NO: 242 - protein, 7 aa, Artificial Sequence
  Artificial peptide sequence
SDRPYLI

SEQ ID NO: 243 - protein, 7 aa, Artificial Sequence
  Artificial peptide sequence
SDRPYLV

SEQ ID NO: 244 - protein, 7 aa, Artificial Sequence
  Artificial peptide sequence
SDRPYVL

SEQ ID NO: 245 - protein, 7 aa, Artificial Sequence
  Artificial peptide sequence
SDRPYVI

SEQ ID NO: 246 - protein, 7 aa, Artificial Sequence
  Artificial peptide sequence
SDRPYVV

SEQ ID NO: 247 - protein, 7 aa, Artificial Sequence
  Artificial peptide sequence
SDRPWIL

SEQ ID NO: 248 - protein, 7 aa, Artificial Sequence
  Artificial peptide sequence
SDRPWII

SEQ ID NO: 249 - protein, 7 aa, Artificial Sequence
  Artificial peptide sequence
SDRPWIV

SEQ ID NO: 250 - protein, 7 aa, Artificial Sequence
  Artificial peptide sequence
SDRPWLL

SEQ ID NO: 251 - protein, 7 aa, Artificial Sequence
  Artificial peptide sequence
SDRPWLI

SEQ ID NO: 252 - protein, 7 aa, Artificial Sequence
  Artificial peptide sequence
SDRPWLV

SEQ ID NO: 253 - protein, 7 aa, Artificial Sequence
  Artificial peptide sequence
SDRPWVL

SEQ ID NO: 254 - protein, 7 aa, Artificial Sequence
  Artificial peptide sequence
SDRPWVI

SEQ ID NO: 255 - protein, 7 aa, Artificial Sequence
  Artificial peptide sequence
SDRPWVV

SEQ ID NO: 256 - protein, 7 aa, Artificial Sequence
  Artificial peptide sequence
SEKPYIL

SEQ ID NO: 257 - protein, 7 aa, Artificial Sequence
  Artificial peptide sequence
SEKPYII

SEQ ID NO: 258 - protein, 7 aa, Artificial Sequence
  Artificial peptide sequence
SEKPYIV

SEQ ID NO: 259 - protein, 7 aa, Artificial Sequence
  Artificial peptide sequence
SEKPYLL

SEQ ID NO: 260 - protein, 7 aa, Artificial Sequence
  Artificial peptide sequence
SEKPYLI

SEQ ID NO: 261 - protein, 7 aa, Artificial Sequence
  Artificial peptide sequence
SEKPYLV

SEQ ID NO: 262 - protein, 7 aa, Artificial Sequence
  Artificial peptide sequence
SEKPYVL

SEQ ID NO: 263 - protein, 7 aa, Artificial Sequence
  Artificial peptide sequence
SEKPYVI

SEQ ID NO: 264 - protein, 7 aa, Artificial Sequence
  Artificial peptide sequence
SEKPYVV

SEQ ID NO: 265 - protein, 7 aa, Artificial Sequence
  Artificial peptide sequence
SEKPWIL

SEQ ID NO: 266 - protein, 7 aa, Artificial Sequence
  Artificial peptide sequence
SEKPWII

SEQ ID NO: 267 - protein, 7 aa, Artificial Sequence
  Artificial peptide sequence
SEKPWIV

SEQ ID NO: 268 - protein, 7 aa, Artificial Sequence
  Artificial peptide sequence
SEKPWLL

SEQ ID NO: 269 - protein, 7 aa, Artificial Sequence
  Artificial peptide sequence
SEKPWLI

SEQ ID NO: 270 - protein, 7 aa, Artificial Sequence
  Artificial peptide sequence
SEKPWLV

SEQ ID NO: 271 - protein, 7 aa, Artificial Sequence
  Artificial peptide sequence
SEKPWVL

SEQ ID NO: 272 - protein, 7 aa, Artificial Sequence
  Artificial peptide sequence
SEKPWVI

SEQ ID NO: 273 - protein, 7 aa, Artificial Sequence
  Artificial peptide sequence
SEKPWVV

SEQ ID NO: 274 - protein, 7 aa, Artificial Sequence
  Artificial peptide sequence
SERPYIL

SEQ ID NO: 275 - protein, 7 aa, Artificial Sequence
  Artificial peptide sequence
SERPYII

SEQ ID NO: 276 - protein, 7 aa, Artificial Sequence
  Artificial peptide sequence
SERPYIV

SEQ ID NO: 277 - protein, 7 aa, Artificial Sequence
  Artificial peptide sequence
SERPYLL

SEQ ID NO: 278 - protein, 7 aa, Artificial Sequence
  Artificial peptide sequence
SERPYLI

SEQ ID NO: 279 - protein, 7 aa, Artificial Sequence
  Artificial peptide sequence
SERPYLV

SEQ ID NO: 280 - protein, 7 aa, Artificial Sequence
  Artificial peptide sequence
SERPYVL

SEQ ID NO: 281 - protein, 7 aa, Artificial Sequence
  Artificial peptide sequence
SERPYVI

SEQ ID NO: 282 - protein, 7 aa, Artificial Sequence
  Artificial peptide sequence
SERPYVV

SEQ ID NO: 283 - protein, 7 aa, Artificial Sequence
  Artificial peptide sequence
SERPWIL

SEQ ID NO: 284 - protein, 7 aa, Artificial Sequence
  Artificial peptide sequence
SERPWII

SEQ ID NO: 285 - protein, 7 aa, Artificial Sequence
  Artificial peptide sequence
SERPWIV

SEQ ID NO: 286 - protein, 7 aa, Artificial Sequence
  Artificial peptide sequence
SERPWLL

SEQ ID NO: 287 - protein, 7 aa, Artificial Sequence
  Artificial peptide sequence
SERPWLI

SEQ ID NO: 288 - protein, 7 aa, Artificial Sequence
  Artificial peptide sequence
SERPWLV

SEQ ID NO: 289 - protein, 7 aa, Artificial Sequence
  Artificial peptide sequence
SERPWVL

SEQ ID NO: 290 - protein, 7 aa, Artificial Sequence
  Artificial peptide sequence
SERPWVI

SEQ ID NO: 291 - protein, 7 aa, Artificial Sequence
  Artificial peptide sequence
SERPWVV

SEQ ID NO: 292 - protein, 7 aa, Artificial Sequence
  Artificial peptide sequence
TDKPYIL

SEQ ID NO: 293 - protein, 7 aa, Artificial Sequence
  Artificial peptide sequence
TDKPYII

SEQ ID NO: 294 - protein, 7 aa, Artificial Sequence
  Artificial peptide sequence
TDKPYIV

SEQ ID NO: 295 - protein, 7 aa, Artificial Sequence
  Artificial peptide sequence
TDKPYLL

SEQ ID NO: 296 - protein, 7 aa, Artificial Sequence
  Artificial peptide sequence
TDKPYLI

SEQ ID NO: 297 - protein, 7 aa, Artificial Sequence
  Artificial peptide sequence
TDKPYLV

SEQ ID NO: 298 - protein, 7 aa, Artificial Sequence
  Artificial peptide sequence
TDKPYVL

SEQ ID NO: 299 - protein, 7 aa, Artificial Sequence
  Artificial peptide sequence
TDKPYVI

SEQ ID NO: 300 - protein, 7 aa, Artificial Sequence
  Artificial peptide sequence
TDKPYVV

SEQ ID NO: 301 - protein, 7 aa, Artificial Sequence
  Artificial peptide sequence
TDKPWIL

SEQ ID NO: 302 - protein, 7 aa, Artificial Sequence
  Artificial peptide sequence
TDKPWII

SEQ ID NO: 303 - protein, 7 aa, Artificial Sequence
  Artificial peptide sequence
TDKPWIV

SEQ ID NO: 304 - protein, 7 aa, Artificial Sequence
  Artificial peptide sequence
TDKPWLL

SEQ ID NO: 305 - protein, 7 aa, Artificial Sequence
  Artificial peptide sequence
TDKPWLI

SEQ ID NO: 306 - protein, 7 aa, Artificial Sequence
  Artificial peptide sequence
TDKPWLV

SEQ ID NO: 307 - protein, 7 aa, Artificial Sequence
  Artificial peptide sequence
TDKPWVL

SEQ ID NO: 308 - protein, 7 aa, Artificial Sequence
  Artificial peptide sequence
TDKPWVI

SEQ ID NO: 309 - protein, 7 aa, Artificial Sequence
  Artificial peptide sequence
TDKPWVV

SEQ ID NO: 310 - protein, 7 aa, Artificial Sequence
  Artificial peptide sequence
TDRPYIL

SEQ ID NO: 311 - protein, 7 aa, Artificial Sequence
  Artificial peptide sequence
TDRPYII

SEQ ID NO: 312 - protein, 7 aa, Artificial Sequence
  Artificial peptide sequence
TDRPYIV

SEQ ID NO: 313 - protein, 7 aa, Artificial Sequence
  Artificial peptide sequence
TDRPYLL

SEQ ID NO: 314 - protein, 7 aa, Artificial Sequence
  Artificial peptide sequence
TDRPYLI

SEQ ID NO: 315 - protein, 7 aa, Artificial Sequence
  Artificial peptide sequence
TDRPYLV

SEQ ID NO: 316 - protein, 7 aa, Artificial Sequence
  Artificial peptide sequence
TDRPYVL

SEQ ID NO: 317 - protein, 7 aa, Artificial Sequence
  Artificial peptide sequence
TDRPYVI

SEQ ID NO: 318 - protein, 7 aa, Artificial Sequence
  Artificial peptide sequence
TDRPYVV

SEQ ID NO: 319 - protein, 7 aa, Artificial Sequence
  Artificial peptide sequence
TDRPWIL

SEQ ID NO: 320 - protein, 7 aa, Artificial Sequence
  Artificial peptide sequence
TDRPWII

SEQ ID NO: 321 - protein, 7 aa, Artificial Sequence
  Artificial peptide sequence
TDRPWIV

SEQ ID NO: 322 - protein, 7 aa, Artificial Sequence
  Artificial peptide sequence
TDRPWLL

SEQ ID NO: 323 - protein, 7 aa, Artificial Sequence
  Artificial peptide sequence
TDRPWLI

SEQ ID NO: 324 - protein, 7 aa, Artificial Sequence
  Artificial peptide sequence
TDRPWLV

SEQ ID NO: 325 - protein, 7 aa, Artificial Sequence
  Artificial peptide sequence
TDRPWVL

SEQ ID NO: 326 - protein, 7 aa, Artificial Sequence
  Artificial peptide sequence
TDRPWVI

SEQ ID NO: 327 - protein, 7 aa, Artificial Sequence
  Artificial peptide sequence
TDRPWVV

SEQ ID NO: 328 - protein, 7 aa, Artificial Sequence
  Artificial peptide sequence
TEKPYIL

SEQ ID NO: 329 - protein, 7 aa, Artificial Sequence
  Artificial peptide sequence
TEKPYII

SEQ ID NO: 330 - protein, 7 aa, Artificial Sequence
  Artificial peptide sequence
TEKPYIV

SEQ ID NO: 331 - protein, 7 aa, Artificial Sequence
  Artificial peptide sequence
TEKPYLL

SEQ ID NO: 332 - protein, 7 aa, Artificial Sequence
  Artificial peptide sequence
TEKPYLI

SEQ ID NO: 333 - protein, 7 aa, Artificial Sequence
  Artificial peptide sequence
TEKPYLV

SEQ ID NO: 334 - protein, 7 aa, Artificial Sequence
  Artificial peptide sequence
TEKPYVL

SEQ ID NO: 335 - protein, 7 aa, Artificial Sequence
  Artificial peptide sequence
TEKPYVI

SEQ ID NO: 336 - protein, 7 aa, Artificial Sequence
  Artificial peptide sequence
TEKPYVV

SEQ ID NO: 337 - protein, 7 aa, Artificial Sequence
  Artificial peptide sequence
TEKPWIL

SEQ ID NO: 338 - protein, 7 aa, Artificial Sequence
  Artificial peptide sequence
TEKPWII

SEQ ID NO: 339 - protein, 7 aa, Artificial Sequence
  Artificial peptide sequence
TEKPWIV

SEQ ID NO: 340 - protein, 7 aa, Artificial Sequence
  Artificial peptide sequence
TEKPWLL

SEQ ID NO: 341 - protein, 7 aa, Artificial Sequence
  Artificial peptide sequence
TEKPWLI

SEQ ID NO: 342 - protein, 7 aa, Artificial Sequence
  Artificial peptide sequence
TEKPWLV

SEQ ID NO: 343 - protein, 7 aa, Artificial Sequence
  Artificial peptide sequence
TEKPWVL

SEQ ID NO: 344 - protein, 7 aa, Artificial Sequence
  Artificial peptide sequence
TEKPWVI

SEQ ID NO: 345 - protein, 7 aa, Artificial Sequence
  Artificial peptide sequence
TEKPWVV

SEQ ID NO: 346 - protein, 7 aa, Artificial Sequence
  Artificial peptide sequence
TERPYIL

SEQ ID NO: 347 - protein, 7 aa, Artificial Sequence
  Artificial peptide sequence
TERPYII

SEQ ID NO: 348 - protein, 7 aa, Artificial Sequence
  Artificial peptide sequence
TERPYIV

SEQ ID NO: 349 - protein, 7 aa, Artificial Sequence
  Artificial peptide sequence
TERPYLL

SEQ ID NO: 350 - protein, 7 aa, Artificial Sequence
  Artificial peptide sequence
TERPYLI

SEQ ID NO: 351 - protein, 7 aa, Artificial Sequence
  Artificial peptide sequence
TERPYLV

SEQ ID NO: 352 - protein, 7 aa, Artificial Sequence
  Artificial peptide sequence
TERPYVL

SEQ ID NO: 353 - protein, 7 aa, Artificial Sequence
  Artificial peptide sequence
TERPYVI

SEQ ID NO: 354 - protein, 7 aa, Artificial Sequence
  Artificial peptide sequence
TERPYVV

SEQ ID NO: 355 - protein, 7 aa, Artificial Sequence
  Artificial peptide sequence
TERPWIL

SEQ ID NO: 356 - protein, 7 aa, Artificial Sequence
  Artificial peptide sequence
TERPWII

SEQ ID NO: 357 - protein, 7 aa, Artificial Sequence
  Artificial peptide sequence
TERPWIV

SEQ ID NO: 358 - protein, 7 aa, Artificial Sequence
  Artificial peptide sequence
TERPWLL

SEQ ID NO: 359 - protein, 7 aa, Artificial Sequence
  Artificial peptide sequence
TERPWLI

SEQ ID NO: 360 - protein, 7 aa, Artificial Sequence
  Artificial peptide sequence
TERPWLV

SEQ ID NO: 361 - protein, 7 aa, Artificial Sequence
  Artificial peptide sequence
TERPWVL

SEQ ID NO: 362 - protein, 7 aa, Artificial Sequence
  Artificial peptide sequence
TERPWVI

SEQ ID NO: 363 - protein, 7 aa, Artificial Sequence
  Artificial peptide sequence
TERPWVV

SEQ ID NO: 364 - protein, 8 aa, Artificial Sequence
  Artificial peptide sequence
ASDKPYII

SEQ ID NO: 365 - protein, 8 aa, Artificial Sequence
  Artificial peptide sequence
ASDKPYIV

SEQ ID NO: 366 - protein, 8 aa, Artificial Sequence
  Artificial peptide sequence
ASDKPYLL

SEQ ID NO: 367 - protein, 8 aa, Artificial Sequence
  Artificial peptide sequence
ASDKPYLI

SEQ ID NO: 368 - protein, 8 aa, Artificial Sequence
  Artificial peptide sequence
ASDKPYLV

SEQ ID NO: 369 - protein, 8 aa, Artificial Sequence
  Artificial peptide sequence
ASDKPYVL

SEQ ID NO: 370 - protein, 8 aa, Artificial Sequence
  Artificial peptide sequence
ASDKPYVI

SEQ ID NO: 371 - protein, 8 aa, Artificial Sequence
  Artificial peptide sequence
ASDKPYVV

SEQ ID NO: 372 - protein, 8 aa, Artificial Sequence
  Artificial peptide sequence
ASDKPWIL

SEQ ID NO: 373 - protein, 8 aa, Artificial Sequence
  Artificial peptide sequence
ASDKPWII

SEQ ID NO: 374 - protein, 8 aa, Artificial Sequence
  Artificial peptide sequence
ASDKPWIV

SEQ ID NO: 375 - protein, 8 aa, Artificial Sequence
  Artificial peptide sequence
ASDKPWLL

SEQ ID NO: 376 - protein, 8 aa, Artificial Sequence
  Artificial peptide sequence
ASDKPWLI

SEQ ID NO: 377 - protein, 8 aa, Artificial Sequence
  Artificial peptide sequence
ASDKPWLV

SEQ ID NO: 378 - protein, 8 aa, Artificial Sequence
  Artificial peptide sequence
ASDKPWVL

SEQ ID NO: 379 - protein, 8 aa, Artificial Sequence
  Artificial peptide sequence
ASDKPWVI

SEQ ID NO: 380 - protein, 8 aa, Artificial Sequence
  Artificial peptide sequence
ASDKPWVV

SEQ ID NO: 381 - protein, 8 aa, Artificial Sequence
  Artificial peptide sequence
ASDRPYIL

SEQ ID NO: 382 - protein, 8 aa, Artificial Sequence
  Artificial peptide sequence
ASDRPYII

SEQ ID NO: 383 - protein, 8 aa, Artificial Sequence
  Artificial peptide sequence
ASDRPYIV

SEQ ID NO: 384 - protein, 8 aa, Artificial Sequence
  Artificial peptide sequence
ASDRPYLL

SEQ ID NO: 385 - protein, 8 aa, Artificial Sequence
  Artificial peptide sequence
ASDRPYLI

SEQ ID NO: 386 - protein, 8 aa, Artificial Sequence
  Artificial peptide sequence
ASDRPYLV

SEQ ID NO: 387 - protein, 8 aa, Artificial Sequence
  Artificial peptide sequence
ASDRPYVL

SEQ ID NO: 388 - protein, 8 aa, Artificial Sequence
  Artificial peptide sequence
ASDRPYVI

SEQ ID NO: 389 - protein, 8 aa, Artificial Sequence
  Artificial peptide sequence
ASDRPYVV

SEQ ID NO: 390 - protein, 8 aa, Artificial Sequence
  Artificial peptide sequence
ASDRPWIL

SEQ ID NO: 391 - protein, 8 aa, Artificial Sequence
  Artificial peptide sequence
ASDRPWII

SEQ ID NO: 392 - protein, 8 aa, Artificial Sequence
  Artificial peptide sequence
ASDRPWIV

SEQ ID NO: 393 - protein, 8 aa, Artificial Sequence
  Artificial peptide sequence
ASDRPWLL

SEQ ID NO: 394 - protein, 8 aa, Artificial Sequence
  Artificial peptide sequence
ASDRPWLI

SEQ ID NO: 395 - protein, 8 aa, Artificial Sequence
  Artificial peptide sequence
ASDRPWLV

SEQ ID NO: 396 - protein, 8 aa, Artificial Sequence
  Artificial peptide sequence
ASDRPWVL

SEQ ID NO: 397 - protein, 8 aa, Artificial Sequence
  Artificial peptide sequence
ASDRPWVI

SEQ ID NO: 398 - protein, 8 aa, Artificial Sequence
  Artificial peptide sequence
ASDRPWVV

SEQ ID NO: 399 - protein, 8 aa, Artificial Sequence
  Artificial peptide sequence
ASEKPYIL

SEQ ID NO: 400 - protein, 8 aa, Artificial Sequence
  Artificial peptide sequence
ASEKPYII

SEQ ID NO: 401 - protein, 8 aa, Artificial Sequence
  Artificial peptide sequence
ASEKPYIV

SEQ ID NO: 402 - protein, 8 aa, Artificial Sequence
  Artificial peptide sequence
ASEKPYLL

SEQ ID NO: 403 - protein, 8 aa, Artificial Sequence
  Artificial peptide sequence
ASEKPYLI

SEQ ID NO: 404 - protein, 8 aa, Artificial Sequence
  Artificial peptide sequence
ASEKPYLV

SEQ ID NO: 405 - protein, 8 aa, Artificial Sequence
  Artificial peptide sequence
ASEKPYVL

SEQ ID NO: 406 - protein, 8 aa, Artificial Sequence
  Artificial peptide sequence
ASEKPYVI

SEQ ID NO: 407 - protein, 8 aa, Artificial Sequence
  Artificial peptide sequence
ASEKPYVV

SEQ ID NO: 408 - protein, 8 aa, Artificial Sequence
  Artificial peptide sequence
ASEKPWIL

SEQ ID NO: 409 - protein, 8 aa, Artificial Sequence
  Artificial peptide sequence
ASEKPWII

SEQ ID NO: 410 - protein, 8 aa, Artificial Sequence
  Artificial peptide sequence
ASEKPWIV

SEQ ID NO: 411 - protein, 8 aa, Artificial Sequence
  Artificial peptide sequence
ASEKPWLL

SEQ ID NO: 412 - protein, 8 aa, Artificial Sequence
  Artificial peptide sequence
ASEKPWLI

SEQ ID NO: 413 - protein, 8 aa, Artificial Sequence
  Artificial peptide sequence
ASEKPWLV

SEQ ID NO: 414 - protein, 8 aa, Artificial Sequence
  Artificial peptide sequence
ASEKPWVL

SEQ ID NO: 415 - protein, 8 aa, Artificial Sequence
  Artificial peptide sequence
ASEKPWVI

SEQ ID NO: 416 - protein, 8 aa, Artificial Sequence
  Artificial peptide sequence
ASEKPWVV

SEQ ID NO: 417 - protein, 8 aa, Artificial Sequence
  Artificial peptide sequence
ASERPYIL

SEQ ID NO: 418 - protein, 8 aa, Artificial Sequence
  Artificial peptide sequence
ASERPYII

SEQ ID NO: 419 - protein, 8 aa, Artificial Sequence
  Artificial peptide sequence
ASERPYIV

SEQ ID NO: 420 - protein, 8 aa, Artificial Sequence
  Artificial peptide sequence
ASERPYLL

SEQ ID NO: 421 - protein, 8 aa, Artificial Sequence
  Artificial peptide sequence
ASERPYLI

SEQ ID NO: 422 - protein, 8 aa, Artificial Sequence
  Artificial peptide sequence
ASERPYLV

SEQ ID NO: 423 - protein, 8 aa, Artificial Sequence
  Artificial peptide sequence
ASERPYVL

SEQ ID NO: 424 - protein, 8 aa, Artificial Sequence
  Artificial peptide sequence
ASERPYVI

SEQ ID NO: 425 - protein, 8 aa, Artificial Sequence
  Artificial peptide sequence
ASERPYVV

SEQ ID NO: 426 - protein, 8 aa, Artificial Sequence
  Artificial peptide sequence
ASERPWIL

SEQ ID NO: 427 - protein, 8 aa, Artificial Sequence
  Artificial peptide sequence
ASERPWII

SEQ ID NO: 428 - protein, 8 aa, Artificial Sequence
  Artificial peptide sequence
ASERPWIV

SEQ ID NO: 429 - protein, 8 aa, Artificial Sequence
  Artificial peptide sequence
ASERPWLL

SEQ ID NO: 430 - protein, 8 aa, Artificial Sequence
  Artificial peptide sequence
ASERPWLI

SEQ ID NO: 431 - protein, 8 aa, Artificial Sequence
  Artificial peptide sequence
ASERPWLV

SEQ ID NO: 432 - protein, 8 aa, Artificial Sequence
  Artificial peptide sequence
ASERPWVL

SEQ ID NO: 433 - protein, 8 aa, Artificial Sequence
  Artificial peptide sequence
ASERPWVI

SEQ ID NO: 434 - protein, 8 aa, Artificial Sequence
  Artificial peptide sequence
ASERPWVV

SEQ ID NO: 435 - protein, 8 aa, Artificial Sequence
  Artificial peptide sequence
ATDKPYIL

SEQ ID NO: 436 - protein, 8 aa, Artificial Sequence
  Artificial peptide sequence
ATDKPYII

SEQ ID NO: 437 - protein, 8 aa, Artificial Sequence
  Artificial peptide sequence
ATDKPYIV

SEQ ID NO: 438 - protein, 8 aa, Artificial Sequence
  Artificial peptide sequence
ATDKPYLL

SEQ ID NO: 439 - protein, 8 aa, Artificial Sequence
  Artificial peptide sequence
ATDKPYLI

SEQ ID NO: 440 - protein, 8 aa, Artificial Sequence
  Artificial peptide sequence
ATDKPYLV

SEQ ID NO: 441 - protein, 8 aa, Artificial Sequence
  Artificial peptide sequence
ATDKPYVL

SEQ ID NO: 442 - protein, 8 aa, Artificial Sequence
  Artificial peptide sequence
ATDKPYVI

SEQ ID NO: 443 - protein, 8 aa, Artificial Sequence
  Artificial peptide sequence
ATDKPYVV

SEQ ID NO: 444 - protein, 8 aa, Artificial Sequence
  Artificial peptide sequence
ATDKPWIL

SEQ ID NO: 445 - protein, 8 aa, Artificial Sequence
  Artificial peptide sequence
ATDKPWII

SEQ ID NO: 446 - protein, 8 aa, Artificial Sequence
  Artificial peptide sequence
ATDKPWIV

SEQ ID NO: 447 - protein, 8 aa, Artificial Sequence
  Artificial peptide sequence
ATDKPWLL

SEQ ID NO: 448 - protein, 8 aa, Artificial Sequence
  Artificial peptide sequence
ATDKPWLI

SEQ ID NO: 449 - protein, 8 aa, Artificial Sequence
  Artificial peptide sequence
ATDKPWLV

SEQ ID NO: 450 - protein, 8 aa, Artificial Sequence
  Artificial peptide sequence
ATDKPWVL

SEQ ID NO: 451 - protein, 8 aa, Artificial Sequence
  Artificial peptide sequence
ATDKPWVI

SEQ ID NO: 452 - protein, 8 aa, Artificial Sequence
  Artificial peptide sequence
ATDKPWVV

SEQ ID NO: 453 - protein, 8 aa, Artificial Sequence
  Artificial peptide sequence
ATDRPYIL

SEQ ID NO: 454 - protein, 8 aa, Artificial Sequence
  Artificial peptide sequence
ATDRPYII

SEQ ID NO: 455 - protein, 8 aa, Artificial Sequence
  Artificial peptide sequence
ATDRPYIV

SEQ ID NO: 456 - protein, 8 aa, Artificial Sequence
  Artificial peptide sequence
ATDRPYLL

SEQ ID NO: 457 - protein, 8 aa, Artificial Sequence
  Artificial peptide sequence
ATDRPYLI

SEQ ID NO: 458 - protein, 8 aa, Artificial Sequence
  Artificial peptide sequence
ATDRPYLV

SEQ ID NO: 459 - protein, 8 aa, Artificial Sequence
  Artificial peptide sequence
ATDRPYVL

SEQ ID NO: 460 - protein, 8 aa, Artificial Sequence
  Artificial peptide sequence
ATDRPYVI

SEQ ID NO: 461 - protein, 8 aa, Artificial Sequence
  Artificial peptide sequence
ATDRPYVV

SEQ ID NO: 462 - protein, 8 aa, Artificial Sequence
  Artificial peptide sequence
ATDRPWIL

SEQ ID NO: 463 - protein, 8 aa, Artificial Sequence
  Artificial peptide sequence
ATDRPWII

SEQ ID NO: 464 - protein, 8 aa, Artificial Sequence
  Artificial peptide sequence
ATDRPWIV

SEQ ID NO: 465 - protein, 8 aa, Artificial Sequence
  Artificial peptide sequence
ATDRPWLL

SEQ ID NO: 466 - protein, 8 aa, Artificial Sequence
  Artificial peptide sequence
ATDRPWLI

SEQ ID NO: 467 - protein, 8 aa, Artificial Sequence
  Artificial peptide sequence
ATDRPWLV

SEQ ID NO: 468 - protein, 8 aa, Artificial Sequence
  Artificial peptide sequence
ATDRPWVL

SEQ ID NO: 469 - protein, 8 aa, Artificial Sequence
  Artificial peptide sequence
ATDRPWVI

SEQ ID NO: 470 - protein, 8 aa, Artificial Sequence
  Artificial peptide sequence
ATDRPWVV

SEQ ID NO: 471 - protein, 8 aa, Artificial Sequence
  Artificial peptide sequence
ATEKPYIL

SEQ ID NO: 472 - protein, 8 aa, Artificial Sequence
  Artificial peptide sequence
ATEKPYII

SEQ ID NO: 473 - protein, 8 aa, Artificial Sequence
  Artificial peptide sequence
ATEKPYIV

SEQ ID NO: 474 - protein, 8 aa, Artificial Sequence
  Artificial peptide sequence
ATEKPYLL

SEQ ID NO: 475 - protein, 8 aa, Artificial Sequence
  Artificial peptide sequence
ATEKPYLI

SEQ ID NO: 476 - protein, 8 aa, Artificial Sequence
  Artificial peptide sequence
ATEKPYLV

SEQ ID NO: 477 - protein, 8 aa, Artificial Sequence
  Artificial peptide sequence
ATEKPYVL

SEQ ID NO: 478 - protein, 8 aa, Artificial Sequence
  Artificial peptide sequence
ATEKPYVI

SEQ ID NO: 479 - protein, 8 aa, Artificial Sequence
  Artificial peptide sequence
ATEKPYVV

SEQ ID NO: 480 - protein, 8 aa, Artificial Sequence
  Artificial peptide sequence
ATEKPWIL

SEQ ID NO: 481 - protein, 8 aa, Artificial Sequence
  Artificial peptide sequence
ATEKPWII

SEQ ID NO: 482 - protein, 8 aa, Artificial Sequence
  Artificial peptide sequence
ATEKPWIV

SEQ ID NO: 483 - protein, 8 aa, Artificial Sequence
  Artificial peptide sequence
ATEKPWLL

SEQ ID NO: 484 - protein, 8 aa, Artificial Sequence
  Artificial peptide sequence
ATEKPWLI

SEQ ID NO: 485 - protein, 8 aa, Artificial Sequence
  Artificial peptide sequence
ATEKPWLV

SEQ ID NO: 486 - protein, 8 aa, Artificial Sequence
  Artificial peptide sequence
ATEKPWVL

SEQ ID NO: 487 - protein, 8 aa, Artificial Sequence
  Artificial peptide sequence
ATEKPWVI

SEQ ID NO: 488 - protein, 8 aa, Artificial Sequence
  Artificial peptide sequence
ATEKPWVV

SEQ ID NO: 489 - protein, 8 aa, Artificial Sequence
  Artificial peptide sequence
ATERPYIL

SEQ ID NO: 490 - protein, 8 aa, Artificial Sequence
  Artificial peptide sequence
ATERPYII

SEQ ID NO: 491 - protein, 8 aa, Artificial Sequence
  Artificial peptide sequence
ATERPYIV

SEQ ID NO: 492 - protein, 8 aa, Artificial Sequence
  Artificial peptide sequence
ATERPYLL

SEQ ID NO: 493 - protein, 8 aa, Artificial Sequence
  Artificial peptide sequence
ATERPYLI

SEQ ID NO: 494 - protein, 8 aa, Artificial Sequence
  Artificial peptide sequence
ATERPYLV

SEQ ID NO: 495 - protein, 8 aa, Artificial Sequence
  Artificial peptide sequence
ATERPYVL

SEQ ID NO: 496 - protein, 8 aa, Artificial Sequence
  Artificial peptide sequence
ATERPYVI

SEQ ID NO: 497 - protein, 8 aa, Artificial Sequence
  Artificial peptide sequence
ATERPYVV

SEQ ID NO: 498 - protein, 8 aa, Artificial Sequence
  Artificial peptide sequence
ATERPWIL

SEQ ID NO: 499 - protein, 8 aa, Artificial Sequence
  Artificial peptide sequence
ATERPWII

SEQ ID NO: 500 - protein, 8 aa, Artificial Sequence
  Artificial peptide sequence
ATERPWIV

SEQ ID NO: 501 - protein, 8 aa, Artificial Sequence
  Artificial peptide sequence
ATERPWLL

SEQ ID NO: 502 - protein, 8 aa, Artificial Sequence
  Artificial peptide sequence
ATERPWLI

SEQ ID NO: 503 - protein, 8 aa, Artificial Sequence
  Artificial peptide sequence
ATERPWLV

SEQ ID NO: 504 - protein, 8 aa, Artificial Sequence
  Artificial peptide sequence
ATERPWVL

SEQ ID NO: 505 - protein, 8 aa, Artificial Sequence
  Artificial peptide sequence
ATERPWVI

SEQ ID NO: 506 - protein, 8 aa, Artificial Sequence
  Artificial peptide sequence
ATERPWVV

SEQ ID NO: 507 - protein, 8 aa, Artificial Sequence
  Artificial peptide sequence
LSDKPYIL

SEQ ID NO: 508 - protein, 8 aa, Artificial Sequence
  Artificial peptide sequence
LSDKPYII

SEQ ID NO: 509 - protein, 8 aa, Artificial Sequence
  Artificial peptide sequence
LSDKPYIV

SEQ ID NO: 510 - protein, 8 aa, Artificial Sequence
  Artificial peptide sequence
LSDKPYLL

SEQ ID NO: 511 - protein, 8 aa, Artificial Sequence
  Artificial peptide sequence
LSDKPYLI

SEQ ID NO: 512 - protein, 8 aa, Artificial Sequence
  Artificial peptide sequence
LSDKPYLV

SEQ ID NO: 513 - protein, 8 aa, Artificial Sequence
  Artificial peptide sequence
LSDKPYVL

SEQ ID NO: 514 - protein, 8 aa, Artificial Sequence
  Artificial peptide sequence
LSDKPYVI

SEQ ID NO: 515 - protein, 8 aa, Artificial Sequence
  Artificial peptide sequence
LSDKPYVV

SEQ ID NO: 516 - protein, 8 aa, Artificial Sequence
  Artificial peptide sequence
LSDKPWIL

SEQ ID NO: 517 - protein, 8 aa, Artificial Sequence
  Artificial peptide sequence
LSDKPWII

SEQ ID NO: 518 - protein, 8 aa, Artificial Sequence
  Artificial peptide sequence
LSDKPWIV

SEQ ID NO: 519 - protein, 8 aa, Artificial Sequence
  Artificial peptide sequence
LSDKPWLL

SEQ ID NO: 520 - protein, 8 aa, Artificial Sequence
  Artificial peptide sequence
LSDKPWLI

SEQ ID NO: 521 - protein, 8 aa, Artificial Sequence
  Artificial peptide sequence
LSDKPWLV

SEQ ID NO: 522 - protein, 8 aa, Artificial Sequence
  Artificial peptide sequence
LSDKPWVL

SEQ ID NO: 523 - protein, 8 aa, Artificial Sequence
  Artificial peptide sequence
LSDKPWVI

SEQ ID NO: 524 - protein, 8 aa, Artificial Sequence
  Artificial peptide sequence
LSDKPWVV

SEQ ID NO: 525 - protein, 8 aa, Artificial Sequence
  Artificial peptide sequence
LSDRPYIL

SEQ ID NO: 526 - protein, 8 aa, Artificial Sequence
  Artificial peptide sequence
LSDRPYII

SEQ ID NO: 527 - protein, 8 aa, Artificial Sequence
  Artificial peptide sequence
LSDRPYIV

SEQ ID NO: 528 - protein, 8 aa, Artificial Sequence
  Artificial peptide sequence
LSDRPYLL

SEQ ID NO: 529 - protein, 8 aa, Artificial Sequence
  Artificial peptide sequence
LSDRPYLI

SEQ ID NO: 530 - protein, 8 aa, Artificial Sequence
  Artificial peptide sequence
LSDRPYLV

SEQ ID NO: 531 - protein, 8 aa, Artificial Sequence
  Artificial peptide sequence
LSDRPYVL

SEQ ID NO: 532 - protein, 8 aa, Artificial Sequence
  Artificial peptide sequence
LSDRPYVI

SEQ ID NO: 533 - protein, 8 aa, Artificial Sequence
  Artificial peptide sequence
LSDRPYVV

SEQ ID NO: 534 - protein, 8 aa, Artificial Sequence
  Artificial peptide sequence
LSDRPWIL

SEQ ID NO: 535 - protein, 8 aa, Artificial Sequence
  Artificial peptide sequence
LSDRPWII

SEQ ID NO: 536 - protein, 8 aa, Artificial Sequence
  Artificial peptide sequence
LSDRPWIV

SEQ ID NO: 537 - protein, 8 aa, Artificial Sequence
  Artificial peptide sequence
LSDRPWLL

SEQ ID NO: 538 - protein, 8 aa, Artificial Sequence
  Artificial peptide sequence
LSDRPWLI

SEQ ID NO: 539 - protein, 8 aa, Artificial Sequence
  Artificial peptide sequence
LSDRPWLV

SEQ ID NO: 540 - protein, 8 aa, Artificial Sequence
  Artificial peptide sequence
LSDRPWVL

SEQ ID NO: 541 - protein, 8 aa, Artificial Sequence
  Artificial peptide sequence
LSDRPWVI

SEQ ID NO: 542 - protein, 8 aa, Artificial Sequence
  Artificial peptide sequence
LSDRPWVV

SEQ ID NO: 543 - protein, 8 aa, Artificial Sequence
  Artificial peptide sequence
LSEKPYIL

SEQ ID NO: 544 - protein, 8 aa, Artificial Sequence
  Artificial peptide sequence
LSEKPYII

SEQ ID NO: 545 - protein, 8 aa, Artificial Sequence
  Artificial peptide sequence
LSEKPYIV

SEQ ID NO: 546 - protein, 8 aa, Artificial Sequence
  Artificial peptide sequence
LSEKPYLL

SEQ ID NO: 547 - protein, 8 aa, Artificial Sequence
  Artificial peptide sequence
LSEKPYLI

SEQ ID NO: 548 - protein, 8 aa, Artificial Sequence
  Artificial peptide sequence
LSEKPYLV

SEQ ID NO: 549 - protein, 8 aa, Artificial Sequence
  Artificial peptide sequence
LSEKPYVL

SEQ ID NO: 550 - protein, 8 aa, Artificial Sequence
  Artificial peptide sequence
LSEKPYVI

SEQ ID NO: 551 - protein, 8 aa, Artificial Sequence
  Artificial peptide sequence
LSEKPYVV

SEQ ID NO: 552 - protein, 8 aa, Artificial Sequence
  Artificial peptide sequence
LSEKPWIL

SEQ ID NO: 553 - protein, 8 aa, Artificial Sequence
  Artificial peptide sequence
LSEKPWII

SEQ ID NO: 554 - protein, 8 aa, Artificial Sequence
  Artificial peptide sequence
LSEKPWIV

SEQ ID NO: 555 - protein, 8 aa, Artificial Sequence
  Artificial peptide sequence
LSEKPWLL

SEQ ID NO: 556 - protein, 8 aa, Artificial Sequence
  Artificial peptide sequence
LSEKPWLI

SEQ ID NO: 557 - protein, 8 aa, Artificial Sequence
  Artificial peptide sequence
LSEKPWLV

SEQ ID NO: 558 - protein, 8 aa, Artificial Sequence
  Artificial peptide sequence
LSEKPWVL

SEQ ID NO: 559 - protein, 8 aa, Artificial Sequence
  Artificial peptide sequence
LSEKPWVI

SEQ ID NO: 560 - protein, 8 aa, Artificial Sequence
  Artificial peptide sequence
LSEKPWVV

SEQ ID NO: 561 - protein, 8 aa, Artificial Sequence
  Artificial peptide sequence
LSERPYIL

SEQ ID NO: 562 - protein, 8 aa, Artificial Sequence
  Artificial peptide sequence
LSERPYII

SEQ ID NO: 563 - protein, 8 aa, Artificial Sequence
  Artificial peptide sequence
LSERPYIV

SEQ ID NO: 564 - protein, 8 aa, Artificial Sequence
  Artificial peptide sequence
LSERPYLL

SEQ ID NO: 565 - protein, 8 aa, Artificial Sequence
  Artificial peptide sequence
LSERPYLI

SEQ ID NO: 566 - protein, 8 aa, Artificial Sequence
  Artificial peptide sequence
LSERPYLV

SEQ ID NO: 567 - protein, 8 aa, Artificial Sequence
  Artificial peptide sequence
LSERPYVL

SEQ ID NO: 568 - protein, 8 aa, Artificial Sequence
  Artificial peptide sequence
LSERPYVI

SEQ ID NO: 569 - protein, 8 aa, Artificial Sequence
  Artificial peptide sequence
LSERPYVV

SEQ ID NO: 570 - protein, 8 aa, Artificial Sequence
  Artificial peptide sequence
LSERPWIL

SEQ ID NO: 571 - protein, 8 aa, Artificial Sequence
  Artificial peptide sequence
LSERPWII

SEQ ID NO: 572 - protein, 8 aa, Artificial Sequence
  Artificial peptide sequence
LSERPWIV

SEQ ID NO: 573 - protein, 8 aa, Artificial Sequence
  Artificial peptide sequence
LSERPWLL

SEQ ID NO: 574 - protein, 8 aa, Artificial Sequence
  Artificial peptide sequence
LSERPWLI

SEQ ID NO: 575 - protein, 8 aa, Artificial Sequence
  Artificial peptide sequence
LSERPWLV

SEQ ID NO: 576 - protein, 8 aa, Artificial Sequence
  Artificial peptide sequence
LSERPWVL

SEQ ID NO: 577 - protein, 8 aa, Artificial Sequence
  Artificial peptide sequence
LSERPWVI

SEQ ID NO: 578 - protein, 8 aa, Artificial Sequence
  Artificial peptide sequence
LSERPWVV

SEQ ID NO: 579 - protein, 8 aa, Artificial Sequence
  Artificial peptide sequence
LTDKPYIL

SEQ ID NO: 580 - protein, 8 aa, Artificial Sequence
  Artificial peptide sequence
LTDKPYII

SEQ ID NO: 581 - protein, 8 aa, Artificial Sequence
  Artificial peptide sequence
LTDKPYIV

SEQ ID NO: 582 - protein, 8 aa, Artificial Sequence
  Artificial peptide sequence
LTDKPYLL

SEQ ID NO: 583 - protein, 8 aa, Artificial Sequence
  Artificial peptide sequence
LTDKPYLI

SEQ ID NO: 584 - protein, 8 aa, Artificial Sequence
  Artificial peptide sequence
LTDKPYLV

SEQ ID NO: 585 - protein, 8 aa, Artificial Sequence
  Artificial peptide sequence
LTDKPYVL

SEQ ID NO: 586 - protein, 8 aa, Artificial Sequence
  Artificial peptide sequence
LTDKPYVI

SEQ ID NO: 587 - protein, 8 aa, Artificial Sequence
  Artificial peptide sequence
LTDKPYVV

SEQ ID NO: 588 - protein, 8 aa, Artificial Sequence
  Artificial peptide sequence
LTDKPWIL

SEQ ID NO: 589 - protein, 8 aa, Artificial Sequence
  Artificial peptide sequence
LTDKPWII

SEQ ID NO: 590 - protein, 8 aa, Artificial Sequence
  Artificial peptide sequence
LTDKPWIV

SEQ ID NO: 591 - protein, 8 aa, Artificial Sequence
  Artificial peptide sequence
LTDKPWLL

SEQ ID NO: 592 - protein, 8 aa, Artificial Sequence
  Artificial peptide sequence
LTDKPWLI

SEQ ID NO: 593 - protein, 8 aa, Artificial Sequence
  Artificial peptide sequence
LTDKPWLV

SEQ ID NO: 594 - protein, 8 aa, Artificial Sequence
  Artificial peptide sequence
LTDKPWVL

SEQ ID NO: 595 - protein, 8 aa, Artificial Sequence
  Artificial peptide sequence
LTDKPWVI

SEQ ID NO: 596 - protein, 8 aa, Artificial Sequence
  Artificial peptide sequence
LTDKPWVV

SEQ ID NO: 597 - protein, 8 aa, Artificial Sequence
  Artificial peptide sequence
LTDRPYIL

SEQ ID NO: 598 - protein, 8 aa, Artificial Sequence
  Artificial peptide sequence
LTDRPYII

SEQ ID NO: 599 - protein, 8 aa, Artificial Sequence
  Artificial peptide sequence
LTDRPYIV

SEQ ID NO: 600 - protein, 8 aa, Artificial Sequence
  Artificial peptide sequence
LTDRPYLL

SEQ ID NO: 601 - protein, 8 aa, Artificial Sequence
  Artificial peptide sequence
LTDRPYLI

SEQ ID NO: 602 - protein, 8 aa, Artificial Sequence
  Artificial peptide sequence
LTDRPYLV

SEQ ID NO: 603 - protein, 8 aa, Artificial Sequence
  Artificial peptide sequence
LTDRPYVL

SEQ ID NO: 604 - protein, 8 aa, Artificial Sequence
  Artificial peptide sequence
LTDRPYVI

SEQ ID NO: 605 - protein, 8 aa, Artificial Sequence
  Artificial peptide sequence
LTDRPYVV

SEQ ID NO: 606 - protein, 8 aa, Artificial Sequence
  Artificial peptide sequence
LTDRPWIL

SEQ ID NO: 607 - protein, 8 aa, Artificial Sequence
  Artificial peptide sequence
LTDRPWII

SEQ ID NO: 608 - protein, 8 aa, Artificial Sequence
  Artificial peptide sequence
LTDRPWIV

SEQ ID NO: 609 - protein, 8 aa, Artificial Sequence
  Artificial peptide sequence
LTDRPWLL

SEQ ID NO: 610 - protein, 8 aa, Artificial Sequence
  Artificial peptide sequence
LTDRPWLI

SEQ ID NO: 611 - protein, 8 aa, Artificial Sequence
  Artificial peptide sequence
LTDRPWLV

SEQ ID NO: 612 - protein, 8 aa, Artificial Sequence
  Artificial peptide sequence
LTDRPWVL

SEQ ID NO: 613 - protein, 8 aa, Artificial Sequence
  Artificial peptide sequence
LTDRPWVI

SEQ ID NO: 614 - protein, 8 aa, Artificial Sequence
  Artificial peptide sequence
LTDRPWVV

SEQ ID NO: 615 - protein, 8 aa, Artificial Sequence
  Artificial peptide sequence
LTEKPYIL

SEQ ID NO: 616 - protein, 8 aa, Artificial Sequence
  Artificial peptide sequence
LTEKPYII

SEQ ID NO: 617 - protein, 8 aa, Artificial Sequence
  Artificial peptide sequence
LTEKPYIV

SEQ ID NO: 618 - protein, 8 aa, Artificial Sequence
  Artificial peptide sequence
LTEKPYLL

SEQ ID NO: 619 - protein, 8 aa, Artificial Sequence
  Artificial peptide sequence
LTEKPYLI

SEQ ID NO: 620 - protein, 8 aa, Artificial Sequence
  Artificial peptide sequence
LTEKPYLV

SEQ ID NO: 621 - protein, 8 aa, Artificial Sequence
  Artificial peptide sequence
LTEKPYVL

SEQ ID NO: 622 - protein, 8 aa, Artificial Sequence
  Artificial peptide sequence
LTEKPYVI

SEQ ID NO: 623 - protein, 8 aa, Artificial Sequence
  Artificial peptide sequence
LTEKPYVV

SEQ ID NO: 624 - protein, 8 aa, Artificial Sequence
  Artificial peptide sequence
LTEKPWIL

SEQ ID NO: 625 - protein, 8 aa, Artificial Sequence
  Artificial peptide sequence
LTEKPWII

SEQ ID NO: 626 - protein, 8 aa, Artificial Sequence
  Artificial peptide sequence
LTEKPWIV

SEQ ID NO: 627 - protein, 8 aa, Artificial Sequence
  Artificial peptide sequence
LTEKPWLL

SEQ ID NO: 628 - protein, 8 aa, Artificial Sequence
  Artificial peptide sequence
LTEKPWLI

SEQ ID NO: 629 - protein, 8 aa, Artificial Sequence
  Artificial peptide sequence
LTEKPWLV

SEQ ID NO: 630 - protein, 8 aa, Artificial Sequence
  Artificial peptide sequence
LTEKPWVL

SEQ ID NO: 631 - protein, 8 aa, Artificial Sequence
  Artificial peptide sequence
LTEKPWVI

SEQ ID NO: 632 - protein, 8 aa, Artificial Sequence
  Artificial peptide sequence
LTEKPWVV

SEQ ID NO: 633 - protein, 8 aa, Artificial Sequence
  Artificial peptide sequence
LTERPYIL

SEQ ID NO: 634 - protein, 8 aa, Artificial Sequence
  Artificial peptide sequence
LTERPYII

SEQ ID NO: 635 - protein, 8 aa, Artificial Sequence
  Artificial peptide sequence
LTERPYIV

SEQ ID NO: 636 - protein, 8 aa, Artificial Sequence
  Artificial peptide sequence
LTERPYLL

SEQ ID NO: 637 - protein, 8 aa, Artificial Sequence
  Artificial peptide sequence
LTERPYLI

SEQ ID NO: 638 - protein, 8 aa, Artificial Sequence
  Artificial peptide sequence
LTERPYLV

SEQ ID NO: 639 - protein, 8 aa, Artificial Sequence
  Artificial peptide sequence
LTERPYVL

SEQ ID NO: 640 - protein, 8 aa, Artificial Sequence
  Artificial peptide sequence
LTERPYVI

SEQ ID NO: 641 - protein, 8 aa, Artificial Sequence
  Artificial peptide sequence
LTERPYVV

SEQ ID NO: 642 - protein, 8 aa, Artificial Sequence
  Artificial peptide sequence
LTERPWIL

SEQ ID NO: 643 - protein, 8 aa, Artificial Sequence
  Artificial peptide sequence
LTERPWII

SEQ ID NO: 644 - protein, 8 aa, Artificial Sequence
  Artificial peptide sequence
LTERPWIV

SEQ ID NO: 645 - protein, 8 aa, Artificial Sequence
  Artificial peptide sequence
LTERPWLL

SEQ ID NO: 646 - protein, 8 aa, Artificial Sequence
  Artificial peptide sequence
LTERPWLI

SEQ ID NO: 647 - protein, 8 aa, Artificial Sequence
  Artificial peptide sequence
LTERPWLV

SEQ ID NO: 648 - protein, 8 aa, Artificial Sequence
  Artificial peptide sequence
LTERPWVL

SEQ ID NO: 649 - protein, 8 aa, Artificial Sequence
  Artificial peptide sequence
LTERPWVI

SEQ ID NO: 650 - protein, 8 aa, Artificial Sequence
  Artificial peptide sequence
LTERPWVV

SEQ ID NO: 651 - protein, 8 aa, Artificial Sequence
  Artificial peptide sequence
ISDKPYIL

SEQ ID NO: 652 - protein, 8 aa, Artificial Sequence
  Artificial peptide sequence
ISDKPYII

SEQ ID NO: 653 - protein, 8 aa, Artificial Sequence
  Artificial peptide sequence
ISDKPYIV

SEQ ID NO: 654 - protein, 8 aa, Artificial Sequence
  Artificial peptide sequence
ISDKPYLL

SEQ ID NO: 655 - protein, 8 aa, Artificial Sequence
  Artificial peptide sequence
ISDKPYLI

SEQ ID NO: 656 - protein, 8 aa, Artificial Sequence
  Artificial peptide sequence
ISDKPYLV

SEQ ID NO: 657 - protein, 8 aa, Artificial Sequence
  Artificial peptide sequence
ISDKPYVL

SEQ ID NO: 658 - protein, 8 aa, Artificial Sequence
  Artificial peptide sequence
ISDKPYVI

SEQ ID NO: 659 - protein, 8 aa, Artificial Sequence
  Artificial peptide sequence
ISDKPYVV

SEQ ID NO: 660 - protein, 8 aa, Artificial Sequence
  Artificial peptide sequence
ISDKPWIL

SEQ ID NO: 661 - protein, 8 aa, Artificial Sequence
  Artificial peptide sequence
ISDKPWII

SEQ ID NO: 662 - protein, 8 aa, Artificial Sequence
  Artificial peptide sequence
ISDKPWIV

SEQ ID NO: 663 - protein, 8 aa, Artificial Sequence
  Artificial peptide sequence
ISDKPWLL

SEQ ID NO: 664 - protein, 8 aa, Artificial Sequence
  Artificial peptide sequence
ISDKPWLI

SEQ ID NO: 665 - protein, 8 aa, Artificial Sequence
  Artificial peptide sequence
ISDKPWLV

SEQ ID NO: 666 - protein, 8 aa, Artificial Sequence
  Artificial peptide sequence
ISDKPWVL

SEQ ID NO: 667 - protein, 8 aa, Artificial Sequence
  Artificial peptide sequence
ISDKPWVI

SEQ ID NO: 668 - protein, 8 aa, Artificial Sequence
  Artificial peptide sequence
ISDKPWVV

SEQ ID NO: 669 - protein, 8 aa, Artificial Sequence
  Artificial peptide sequence
ISDRPYIL

SEQ ID NO: 670 - protein, 8 aa, Artificial Sequence
  Artificial peptide sequence
ISDRPYII

SEQ ID NO: 671 - protein, 8 aa, Artificial Sequence
  Artificial peptide sequence
ISDRPYIV

SEQ ID NO: 672 - protein, 8 aa, Artificial Sequence
  Artificial peptide sequence
ISDRPYLL

SEQ ID NO: 673 - protein, 8 aa, Artificial Sequence
  Artificial peptide sequence
ISDRPYLI

SEQ ID NO: 674 - protein, 8 aa, Artificial Sequence
  Artificial peptide sequence
ISDRPYLV

SEQ ID NO: 675 - protein, 8 aa, Artificial Sequence
  Artificial peptide sequence
ISDRPYVL

SEQ ID NO: 676 - protein, 8 aa, Artificial Sequence
  Artificial peptide sequence
ISDRPYVI

SEQ ID NO: 677 - protein, 8 aa, Artificial Sequence
  Artificial peptide sequence
ISDRPYVV

SEQ ID NO: 678 - protein, 8 aa, Artificial Sequence
  Artificial peptide sequence
ISDRPWIL

SEQ ID NO: 679 - protein, 8 aa, Artificial Sequence
  Artificial peptide sequence
ISDRPWII

SEQ ID NO: 680 - protein, 8 aa, Artificial Sequence
  Artificial peptide sequence
ISDRPWIV

SEQ ID NO: 681 - protein, 8 aa, Artificial Sequence
  Artificial peptide sequence
ISDRPWLL

SEQ ID NO: 682 - protein, 8 aa, Artificial Sequence
  Artificial peptide sequence
ISDRPWLI

SEQ ID NO: 683 - protein, 8 aa, Artificial Sequence
  Artificial peptide sequence
ISDRPWLV

SEQ ID NO: 684 - protein, 8 aa, Artificial Sequence
  Artificial peptide sequence
ISDRPWVL

SEQ ID NO: 685 - protein, 8 aa, Artificial Sequence
  Artificial peptide sequence
ISDRPWVI

SEQ ID NO: 686 - protein, 8 aa, Artificial Sequence
  Artificial peptide sequence
ISDRPWVV

SEQ ID NO: 687 - protein, 8 aa, Artificial Sequence
  Artificial peptide sequence
ISEKPYIL

SEQ ID NO: 688 - protein, 8 aa, Artificial Sequence
  Artificial peptide sequence
ISEKPYII

SEQ ID NO: 689 - protein, 8 aa, Artificial Sequence
  Artificial peptide sequence
ISEKPYIV

SEQ ID NO: 690 - protein, 8 aa, Artificial Sequence
  Artificial peptide sequence
ISEKPYLL

SEQ ID NO: 691 - protein, 8 aa, Artificial Sequence
  Artificial peptide sequence
ISEKPYLI

SEQ ID NO: 692 - protein, 8 aa, Artificial Sequence
  Artificial peptide sequence
ISEKPYLV

SEQ ID NO: 693 - protein, 8 aa, Artificial Sequence
  Artificial peptide sequence
ISEKPYVL

SEQ ID NO: 694 - protein, 8 aa, Artificial Sequence
  Artificial peptide sequence
ISEKPYVI

SEQ ID NO: 695 - protein, 8 aa, Artificial Sequence
  Artificial peptide sequence
ISEKPYVV

SEQ ID NO: 696 - protein, 8 aa, Artificial Sequence
  Artificial peptide sequence
ISEKPWIL

SEQ ID NO: 697 - protein, 8 aa, Artificial Sequence
  Artificial peptide sequence
ISEKPWII

SEQ ID NO: 698 - protein, 8 aa, Artificial Sequence
  Artificial peptide sequence
ISEKPWIV

SEQ ID NO: 699 - protein, 8 aa, Artificial Sequence
  Artificial peptide sequence
ISEKPWLL

SEQ ID NO: 700 - protein, 8 aa, Artificial Sequence
  Artificial peptide sequence
ISEKPWLI

SEQ ID NO: 701 - protein, 8 aa, Artificial Sequence
  Artificial peptide sequence
ISEKPWLV

SEQ ID NO: 702 - protein, 8 aa, Artificial Sequence
  Artificial peptide sequence
ISEKPWVL

SEQ ID NO: 703 - protein, 8 aa, Artificial Sequence
  Artificial peptide sequence
ISEKPWVI

SEQ ID NO: 704 - protein, 8 aa, Artificial Sequence
  Artificial peptide sequence
ISEKPWVV

SEQ ID NO: 705 - protein, 8 aa, Artificial Sequence
  Artificial peptide sequence
ISERPYIL

SEQ ID NO: 706 - protein, 8 aa, Artificial Sequence
  Artificial peptide sequence
ISERPYII

SEQ ID NO: 707 - protein, 8 aa, Artificial Sequence
  Artificial peptide sequence
ISERPYIV

SEQ ID NO: 708 - protein, 8 aa, Artificial Sequence
  Artificial peptide sequence
ISERPYLL

SEQ ID NO: 709 - protein, 8 aa, Artificial Sequence
  Artificial peptide sequence
ISERPYLI

SEQ ID NO: 710 - protein, 8 aa, Artificial Sequence
  Artificial peptide sequence
ISERPYLV

SEQ ID NO: 711 - protein, 8 aa, Artificial Sequence
  Artificial peptide sequence
ISERPYVL

SEQ ID NO: 712 - protein, 8 aa, Artificial Sequence
  Artificial peptide sequence
ISERPYVI

SEQ ID NO: 713 - protein, 8 aa, Artificial Sequence
  Artificial peptide sequence
ISERPYVV

SEQ ID NO: 714 - protein, 8 aa, Artificial Sequence
  Artificial peptide sequence
ISERPWIL

SEQ ID NO: 715 - protein, 8 aa, Artificial Sequence
  Artificial peptide sequence
ISERPWII

SEQ ID NO: 716 - protein, 8 aa, Artificial Sequence
  Artificial peptide sequence
ISERPWIV

SEQ ID NO: 717 - protein, 8 aa, Artificial Sequence
  Artificial peptide sequence
ISERPWLL

SEQ ID NO: 718 - protein, 8 aa, Artificial Sequence
  Artificial peptide sequence
ISERPWLI

SEQ ID NO: 719 - protein, 8 aa, Artificial Sequence
  Artificial peptide sequence
ISERPWLV

SEQ ID NO: 720 - protein, 8 aa, Artificial Sequence
  Artificial peptide sequence
ISERPWVL

SEQ ID NO: 721 - protein, 8 aa, Artificial Sequence
  Artificial peptide sequence
ISERPWVI

SEQ ID NO: 722 - protein, 8 aa, Artificial Sequence
  Artificial peptide sequence
ISERPWVV

SEQ ID NO: 723 - protein, 8 aa, Artificial Sequence
  Artificial peptide sequence
ITDKPYIL

SEQ ID NO: 724 - protein, 8 aa, Artificial Sequence
  Artificial peptide sequence
ITDKPYII

SEQ ID NO: 725 - protein, 8 aa, Artificial Sequence
  Artificial peptide sequence
ITDKPYIV

SEQ ID NO: 726 - protein, 8 aa, Artificial Sequence
  Artificial peptide sequence
ITDKPYLL

SEQ ID NO: 727 - protein, 8 aa, Artificial Sequence
  Artificial peptide sequence
ITDKPYLI

SEQ ID NO: 728 - protein, 8 aa, Artificial Sequence
  Artificial peptide sequence
ITDKPYLV

SEQ ID NO: 729 - protein, 8 aa, Artificial Sequence
  Artificial peptide sequence
ITDKPYVL

SEQ ID NO: 730 - protein, 8 aa, Artificial Sequence
  Artificial peptide sequence
ITDKPYVI

SEQ ID NO: 731 - protein, 8 aa, Artificial Sequence
  Artificial peptide sequence
ITDKPYVV

SEQ ID NO: 732 - protein, 8 aa, Artificial Sequence
  Artificial peptide sequence
ITDKPWIL

SEQ ID NO: 733 - protein, 8 aa, Artificial Sequence
  Artificial peptide sequence
ITDKPWII

SEQ ID NO: 734 - protein, 8 aa, Artificial Sequence
  Artificial peptide sequence
ITDKPWIV

SEQ ID NO: 735 - protein, 8 aa, Artificial Sequence
  Artificial peptide sequence
ITDKPWLL

SEQ ID NO: 736 - protein, 8 aa, Artificial Sequence
  Artificial peptide sequence
ITDKPWLI

SEQ ID NO: 737 - protein, 8 aa, Artificial Sequence
  Artificial peptide sequence
ITDKPWLV

SEQ ID NO: 738 - protein, 8 aa, Artificial Sequence
  Artificial peptide sequence
ITDKPWVL

SEQ ID NO: 739 - protein, 8 aa, Artificial Sequence
  Artificial peptide sequence
ITDKPWVI

SEQ ID NO: 740 - protein, 8 aa, Artificial Sequence
  Artificial peptide sequence
ITDKPWVV

SEQ ID NO: 741 - protein, 8 aa, Artificial Sequence
  Artificial peptide sequence
ITDRPYIL

SEQ ID NO: 742 - protein, 8 aa, Artificial Sequence
  Artificial peptide sequence
ITDRPYII

SEQ ID NO: 743 - protein, 8 aa, Artificial Sequence
  Artificial peptide sequence
ITDRPYIV

SEQ ID NO: 744 - protein, 8 aa, Artificial Sequence
  Artificial peptide sequence
ITDRPYLL

SEQ ID NO: 745 - protein, 8 aa, Artificial Sequence
  Artificial peptide sequence
ITDRPYLI

SEQ ID NO: 746 - protein, 8 aa, Artificial Sequence
  Artificial peptide sequence
ITDRPYLV

SEQ ID NO: 747 - protein, 8 aa, Artificial Sequence
  Artificial peptide sequence
ITDRPYVL

SEQ ID NO: 748 - protein, 8 aa, Artificial Sequence
  Artificial peptide sequence
ITDRPYVI

SEQ ID NO: 749 - protein, 8 aa, Artificial Sequence
  Artificial peptide sequence
ITDRPYVV

SEQ ID NO: 750 - protein, 8 aa, Artificial Sequence
  Artificial peptide sequence
ITDRPWIL

SEQ ID NO: 751 - protein, 8 aa, Artificial Sequence
  Artificial peptide sequence
ITDRPWII

SEQ ID NO: 752 - protein, 8 aa, Artificial Sequence
  Artificial peptide sequence
ITDRPWIV

SEQ ID NO: 753 - protein, 8 aa, Artificial Sequence
  Artificial peptide sequence
ITDRPWLL

SEQ ID NO: 754 - protein, 8 aa, Artificial Sequence
  Artificial peptide sequence
ITDRPWLI

SEQ ID NO: 755 - protein, 8 aa, Artificial Sequence
  Artificial peptide sequence
ITDRPWLV

SEQ ID NO: 756 - protein, 8 aa, Artificial Sequence
  Artificial peptide sequence
ITDRPWVL

SEQ ID NO: 757 - protein, 8 aa, Artificial Sequence
  Artificial peptide sequence
ITDRPWVI

SEQ ID NO: 758 - protein, 8 aa, Artificial Sequence
  Artificial peptide sequence
ITDRPWVV

SEQ ID NO: 759 - protein, 8 aa, Artificial Sequence
  Artificial peptide sequence
ITEKPYIL

SEQ ID NO: 760 - protein, 8 aa, Artificial Sequence
  Artificial peptide sequence
ITEKPYII

SEQ ID NO: 761 - protein, 8 aa, Artificial Sequence
  Artificial peptide sequence
ITEKPYIV

SEQ ID NO: 762 - protein, 8 aa, Artificial Sequence
  Artificial peptide sequence
ITEKPYLL

SEQ ID NO: 763 - protein, 8 aa, Artificial Sequence
  Artificial peptide sequence
ITEKPYLI

SEQ ID NO: 764 - protein, 8 aa, Artificial Sequence
  Artificial peptide sequence
ITEKPYLV

SEQ ID NO: 765 - protein, 8 aa, Artificial Sequence
  Artificial peptide sequence
ITEKPYVL

SEQ ID NO: 766 - protein, 8 aa, Artificial Sequence
  Artificial peptide sequence
ITEKPYVI

SEQ ID NO: 767 - protein, 8 aa, Artificial Sequence
  Artificial peptide sequence
ITEKPYVV

SEQ ID NO: 768 - protein, 8 aa, Artificial Sequence
  Artificial peptide sequence
ITEKPWIL

SEQ ID NO: 769 - protein, 8 aa, Artificial Sequence
  Artificial peptide sequence
ITEKPWII

SEQ ID NO: 770 - protein, 8 aa, Artificial Sequence
  Artificial peptide sequence
ITEKPWIV

SEQ ID NO: 771 - protein, 8 aa, Artificial Sequence
  Artificial peptide sequence
ITEKPWLL

SEQ ID NO: 772 - protein, 8 aa, Artificial Sequence
  Artificial peptide sequence
ITEKPWLI

SEQ ID NO: 773 - protein, 8 aa, Artificial Sequence
  Artificial peptide sequence
ITEKPWLV

SEQ ID NO: 774 - protein, 8 aa, Artificial Sequence
  Artificial peptide sequence
ITEKPWVL

SEQ ID NO: 775 - protein, 8 aa, Artificial Sequence
  Artificial peptide sequence
ITEKPWVI

SEQ ID NO: 776 - protein, 8 aa, Artificial Sequence
  Artificial peptide sequence
ITEKPWVV

SEQ ID NO: 777 - protein, 8 aa, Artificial Sequence
  Artificial peptide sequence
ITERPYIL

SEQ ID NO: 778 - protein, 8 aa, Artificial Sequence
  Artificial peptide sequence
ITERPYII

SEQ ID NO: 779 - protein, 8 aa, Artificial Sequence
  Artificial peptide sequence
ITERPYIV

SEQ ID NO: 780 - protein, 8 aa, Artificial Sequence
  Artificial peptide sequence
ITERPYLL

SEQ ID NO: 781 - protein, 8 aa, Artificial Sequence
  Artificial peptide sequence
ITERPYLI

SEQ ID NO: 782 - protein, 8 aa, Artificial Sequence
  Artificial peptide sequence
ITERPYLV

SEQ ID NO: 783 - protein, 8 aa, Artificial Sequence
  Artificial peptide sequence
ITERPYVL

SEQ ID NO: 784 - protein, 8 aa, Artificial Sequence
  Artificial peptide sequence
ITERPYVI

SEQ ID NO: 785 - protein, 8 aa, Artificial Sequence
  Artificial peptide sequence
ITERPYVV

SEQ ID NO: 786 - protein, 8 aa, Artificial Sequence
  Artificial peptide sequence
ITERPWIL

SEQ ID NO: 787 - protein, 8 aa, Artificial Sequence
  Artificial peptide sequence
ITERPWII

SEQ ID NO: 788 - protein, 8 aa, Artificial Sequence
  Artificial peptide sequence
ITERPWIV

SEQ ID NO: 789 - protein, 8 aa, Artificial Sequence
  Artificial peptide sequence
ITERPWLL

SEQ ID NO: 790 - protein, 8 aa, Artificial Sequence
  Artificial peptide sequence
ITERPWLI

SEQ ID NO: 791 - protein, 8 aa, Artificial Sequence
  Artificial peptide sequence
ITERPWLV

SEQ ID NO: 792 - protein, 8 aa, Artificial Sequence
  Artificial peptide sequence
ITERPWVL

SEQ ID NO: 793 - protein, 8 aa, Artificial Sequence
  Artificial peptide sequence
ITERPWVI

SEQ ID NO: 794 - protein, 8 aa, Artificial Sequence
  Artificial peptide sequence
ITERPWVV

SEQ ID NO: 795 - protein, 8 aa, Artificial Sequence
  Artificial peptide sequence
VSDKPYIL

SEQ ID NO: 796 - protein, 8 aa, Artificial Sequence
  Artificial peptide sequence
VSDKPYII

SEQ ID NO: 797 - protein, 8 aa, Artificial Sequence
  Artificial peptide sequence
VSDKPYIV

SEQ ID NO: 798 - protein, 8 aa, Artificial Sequence
  Artificial peptide sequence
VSDKPYLL

SEQ ID NO: 799 - protein, 8 aa, Artificial Sequence
  Artificial peptide sequence
VSDKPYLI

SEQ ID NO: 800 - protein, 8 aa, Artificial Sequence
  Artificial peptide sequence
VSDKPYLV

SEQ ID NO: 801 - protein, 8 aa, Artificial Sequence
  Artificial peptide sequence
VSDKPYVL

SEQ ID NO: 802 - protein, 8 aa, Artificial Sequence
  Artificial peptide sequence
VSDKPYVI

SEQ ID NO: 803 - protein, 8 aa, Artificial Sequence
  Artificial peptide sequence
VSDKPYVV

SEQ ID NO: 804 - protein, 8 aa, Artificial Sequence
  Artificial peptide sequence
VSDKPWIL

SEQ ID NO: 805 - protein, 8 aa, Artificial Sequence
  Artificial peptide sequence
VSDKPWII

SEQ ID NO: 806 - protein, 8 aa, Artificial Sequence
  Artificial peptide sequence
VSDKPWIV

SEQ ID NO: 807 - protein, 8 aa, Artificial Sequence
  Artificial peptide sequence
VSDKPWLL

SEQ ID NO: 808 - protein, 8 aa, Artificial Sequence
  Artificial peptide sequence
VSDKPWLI

SEQ ID NO: 809 - protein, 8 aa, Artificial Sequence
  Artificial peptide sequence
VSDKPWLV

SEQ ID NO: 810 - protein, 8 aa, Artificial Sequence
  Artificial peptide sequence
VSDKPWVL

SEQ ID NO: 811 - protein, 8 aa, Artificial Sequence
  Artificial peptide sequence
VSDKPWVI

SEQ ID NO: 812 - protein, 8 aa, Artificial Sequence
  Artificial peptide sequence
VSDKPWVV

SEQ ID NO: 813 - protein, 8 aa, Artificial Sequence
  Artificial peptide sequence
VSDRPYIL

SEQ ID NO: 814 - protein, 8 aa, Artificial Sequence
  Artificial peptide sequence
VSDRPYII

SEQ ID NO: 815 - protein, 8 aa, Artificial Sequence
  Artificial peptide sequence
VSDRPYIV

SEQ ID NO: 816 - protein, 8 aa, Artificial Sequence
  Artificial peptide sequence
VSDRPYLL

SEQ ID NO: 817 - protein, 8 aa, Artificial Sequence
  Artificial peptide sequence
VSDRPYLI

SEQ ID NO: 818 - protein, 8 aa, Artificial Sequence
  Artificial peptide sequence
VSDRPYLV

SEQ ID NO: 819 - protein, 8 aa, Artificial Sequence
  Artificial peptide sequence
VSDRPYVL

SEQ ID NO: 820 - protein, 8 aa, Artificial Sequence
  Artificial peptide sequence
VSDRPYVI

SEQ ID NO: 821 - protein, 8 aa, Artificial Sequence
  Artificial peptide sequence
VSDRPYVV

SEQ ID NO: 822 - protein, 8 aa, Artificial Sequence
  Artificial peptide sequence
VSDRPWIL

SEQ ID NO: 823 - protein, 8 aa, Artificial Sequence
  Artificial peptide sequence
VSDRPWII

SEQ ID NO: 824 - protein, 8 aa, Artificial Sequence
  Artificial peptide sequence
VSDRPWIV

SEQ ID NO: 825 - protein, 8 aa, Artificial Sequence
  Artificial peptide sequence
VSDRPWLL

SEQ ID NO: 826 - protein, 8 aa, Artificial Sequence
  Artificial peptide sequence
VSDRPWLI

SEQ ID NO: 827 - protein, 8 aa, Artificial Sequence
  Artificial peptide sequence
VSDRPWLV

SEQ ID NO: 828 - protein, 8 aa, Artificial Sequence
  Artificial peptide sequence
VSDRPWVL

SEQ ID NO: 829 - protein, 8 aa, Artificial Sequence
  Artificial peptide sequence
VSDRPWVI

SEQ ID NO: 830 - protein, 8 aa, Artificial Sequence
  Artificial peptide sequence
VSDRPWVV

SEQ ID NO: 831 - protein, 8 aa, Artificial Sequence
  Artificial peptide sequence
VSEKPYIL

SEQ ID NO: 832 - protein, 8 aa, Artificial Sequence
  Artificial peptide sequence
VSEKPYII

SEQ ID NO: 833 - protein, 8 aa, Artificial Sequence
  Artificial peptide sequence
VSEKPYIV

SEQ ID NO: 834 - protein, 8 aa, Artificial Sequence
  Artificial peptide sequence
VSEKPYLL

SEQ ID NO: 835 - protein, 8 aa, Artificial Sequence
  Artificial peptide sequence
VSEKPYLI

SEQ ID NO: 836 - protein, 8 aa, Artificial Sequence
  Artificial peptide sequence
VSEKPYLV

SEQ ID NO: 837 - protein, 8 aa, Artificial Sequence
  Artificial peptide sequence
VSEKPYVL

SEQ ID NO: 838 - protein, 8 aa, Artificial Sequence
  Artificial peptide sequence
VSEKPYVI

SEQ ID NO: 839 - protein, 8 aa, Artificial Sequence
  Artificial peptide sequence
VSEKPYVV

SEQ ID NO: 840 - protein, 8 aa, Artificial Sequence
  Artificial peptide sequence
VSEKPWIL

SEQ ID NO: 841 - protein, 8 aa, Artificial Sequence
  Artificial peptide sequence
VSEKPWII

SEQ ID NO: 842 - protein, 8 aa, Artificial Sequence
  Artificial peptide sequence
VSEKPWIV

SEQ ID NO: 843 - protein, 8 aa, Artificial Sequence
  Artificial peptide sequence
VSEKPWLL

SEQ ID NO: 844 - protein, 8 aa, Artificial Sequence
  Artificial peptide sequence
VSEKPWLI

SEQ ID NO: 845 - protein, 8 aa, Artificial Sequence
  Artificial peptide sequence
VSEKPWLV

SEQ ID NO: 846 - protein, 8 aa, Artificial Sequence
  Artificial peptide sequence
VSEKPWVL

SEQ ID NO: 847 - protein, 8 aa, Artificial Sequence
  Artificial peptide sequence
VSEKPWVI

SEQ ID NO: 848 - protein, 8 aa, Artificial Sequence
  Artificial peptide sequence
VSEKPWVV

SEQ ID NO: 849 - protein, 8 aa, Artificial Sequence
  Artificial peptide sequence
VSERPYIL

SEQ ID NO: 850 - protein, 8 aa, Artificial Sequence
  Artificial peptide sequence
VSERPYII

SEQ ID NO: 851 - protein, 8 aa, Artificial Sequence
  Artificial peptide sequence
VSERPYIV

SEQ ID NO: 852 - protein, 8 aa, Artificial Sequence
  Artificial peptide sequence
VSERPYLL

SEQ ID NO: 853 - protein, 8 aa, Artificial Sequence
  Artificial peptide sequence
VSERPYLI

SEQ ID NO: 854 - protein, 8 aa, Artificial Sequence
  Artificial peptide sequence
VSERPYLV

SEQ ID NO: 855 - protein, 8 aa, Artificial Sequence
  Artificial peptide sequence
VSERPYVL

SEQ ID NO: 856 - protein, 8 aa, Artificial Sequence
  Artificial peptide sequence
VSERPYVI

SEQ ID NO: 857 - protein, 8 aa, Artificial Sequence
  Artificial peptide sequence
VSERPYVV

SEQ ID NO: 858 - protein, 8 aa, Artificial Sequence
  Artificial peptide sequence
VSERPWIL

SEQ ID NO: 859 - protein, 8 aa, Artificial Sequence
  Artificial peptide sequence
VSERPWII

SEQ ID NO: 860 - protein, 8 aa, Artificial Sequence
  Artificial peptide sequence
VSERPWIV

SEQ ID NO: 861 - protein, 8 aa, Artificial Sequence
  Artificial peptide sequence
VSERPWLL

SEQ ID NO: 862 - protein, 8 aa, Artificial Sequence
  Artificial peptide sequence
VSERPWLI

SEQ ID NO: 863 - protein, 8 aa, Artificial Sequence
  Artificial peptide sequence
VSERPWLV

SEQ ID NO: 864 - protein, 8 aa, Artificial Sequence
  Artificial peptide sequence
VSERPWVL

SEQ ID NO: 865 - protein, 8 aa, Artificial Sequence
  Artificial peptide sequence
VSERPWVI

SEQ ID NO: 866 - protein, 8 aa, Artificial Sequence
  Artificial peptide sequence
VSERPWVV

SEQ ID NO: 867 - protein, 8 aa, Artificial Sequence
  Artificial peptide sequence
VTDKPYIL

SEQ ID NO: 868 - protein, 8 aa, Artificial Sequence
  Artificial peptide sequence
VTDKPYII

SEQ ID NO: 869 - protein, 8 aa, Artificial Sequence
  Artificial peptide sequence
VTDKPYIV

SEQ ID NO: 870 - protein, 8 aa, Artificial Sequence
  Artificial peptide sequence
VTDKPYLL

SEQ ID NO: 871 - protein, 8 aa, Artificial Sequence
  Artificial peptide sequence
VTDKPYLI

SEQ ID NO: 872 - protein, 8 aa, Artificial Sequence
  Artificial peptide sequence
VTDKPYLV

SEQ ID NO: 873 - protein, 8 aa, Artificial Sequence
  Artificial peptide sequence
VTDKPYVL

SEQ ID NO: 874 - protein, 8 aa, Artificial Sequence
  Artificial peptide sequence
VTDKPYVI

SEQ ID NO: 875 - protein, 8 aa, Artificial Sequence
  Artificial peptide sequence
VTDKPYVV

SEQ ID NO: 876 - protein, 8 aa, Artificial Sequence
  Artificial peptide sequence
VTDKPWIL

SEQ ID NO: 877 - protein, 8 aa, Artificial Sequence
  Artificial peptide sequence
VTDKPWII

SEQ ID NO: 878 - protein, 8 aa, Artificial Sequence
  Artificial peptide sequence
VTDKPWIV

SEQ ID NO: 879 - protein, 8 aa, Artificial Sequence
  Artificial peptide sequence
VTDKPWLL

SEQ ID NO: 880 - protein, 8 aa, Artificial Sequence
  Artificial peptide sequence
VTDKPWLI

SEQ ID NO: 881 - protein, 8 aa, Artificial Sequence
  Artificial peptide sequence
VTDKPWLV

SEQ ID NO: 882 - protein, 8 aa, Artificial Sequence
  Artificial peptide sequence
VTDKPWVL

SEQ ID NO: 883 - protein, 8 aa, Artificial Sequence
  Artificial peptide sequence
VTDKPWVI

SEQ ID NO: 884 - protein, 8 aa, Artificial Sequence
  Artificial peptide sequence
VTDKPWVV

SEQ ID NO: 885 - protein, 8 aa, Artificial Sequence
  Artificial peptide sequence
VTDRPYIL

SEQ ID NO: 886 - protein, 8 aa, Artificial Sequence
  Artificial peptide sequence
VTDRPYII

SEQ ID NO: 887 - protein, 8 aa, Artificial Sequence
  Artificial peptide sequence
VTDRPYIV

SEQ ID NO: 888 - protein, 8 aa, Artificial Sequence
  Artificial peptide sequence
VTDRPYLL

SEQ ID NO: 889 - protein, 8 aa, Artificial Sequence
  Artificial peptide sequence
VTDRPYLI

SEQ ID NO: 890 - protein, 8 aa, Artificial Sequence
  Artificial peptide sequence
VTDRPYLV

SEQ ID NO: 891 - protein, 8 aa, Artificial Sequence
  Artificial peptide sequence
VTDRPYVL

SEQ ID NO: 892 - protein, 8 aa, Artificial Sequence
  Artificial peptide sequence
VTDRPYVI

SEQ ID NO: 893 - protein, 8 aa, Artificial Sequence
  Artificial peptide sequence
VTDRPYVV

SEQ ID NO: 894 - protein, 8 aa, Artificial Sequence
  Artificial peptide sequence
VTDRPWIL

SEQ ID NO: 895 - protein, 8 aa, Artificial Sequence
  Artificial peptide sequence
VTDRPWII

SEQ ID NO: 896 - protein, 8 aa, Artificial Sequence
  Artificial peptide sequence
VTDRPWIV

SEQ ID NO: 897 - protein, 8 aa, Artificial Sequence
  Artificial peptide sequence
VTDRPWLL

SEQ ID NO: 898 - protein, 8 aa, Artificial Sequence
  Artificial peptide sequence
VTDRPWLI

SEQ ID NO: 899 - protein, 8 aa, Artificial Sequence
  Artificial peptide sequence
VTDRPWLV

SEQ ID NO: 900 - protein, 8 aa, Artificial Sequence
  Artificial peptide sequence
VTDRPWVL

SEQ ID NO: 901 - protein, 8 aa, Artificial Sequence
  Artificial peptide sequence
VTDRPWVI

SEQ ID NO: 902 - protein, 8 aa, Artificial Sequence
  Artificial peptide sequence
VTDRPWVV

SEQ ID NO: 903 - protein, 8 aa, Artificial Sequence
  Artificial peptide sequence
VTEKPYIL

SEQ ID NO: 904 - protein, 8 aa, Artificial Sequence
  Artificial peptide sequence
VTEKPYII

SEQ ID NO: 905 - protein, 8 aa, Artificial Sequence
  Artificial peptide sequence
VTEKPYIV

SEQ ID NO: 906 - protein, 8 aa, Artificial Sequence
  Artificial peptide sequence
VTEKPYLL

SEQ ID NO: 907 - protein, 8 aa, Artificial Sequence
  Artificial peptide sequence
VTEKPYLI

SEQ ID NO: 908 - protein, 8 aa, Artificial Sequence
  Artificial peptide sequence
VTEKPYLV

SEQ ID NO: 909 - protein, 8 aa, Artificial Sequence
  Artificial peptide sequence
VTEKPYVL

SEQ ID NO: 910 - protein, 8 aa, Artificial Sequence
  Artificial peptide sequence
VTEKPYVI

SEQ ID NO: 911 - protein, 8 aa, Artificial Sequence
  Artificial peptide sequence
VTEKPYVV

SEQ ID NO: 912 - protein, 8 aa, Artificial Sequence
  Artificial peptide sequence
VTEKPWIL

SEQ ID NO: 913 - protein, 8 aa, Artificial Sequence
  Artificial peptide sequence
VTEKPWII

SEQ ID NO: 914 - protein, 8 aa, Artificial Sequence
  Artificial peptide sequence
VTEKPWIV

SEQ ID NO: 915 - protein, 8 aa, Artificial Sequence
  Artificial peptide sequence
VTEKPWLL

SEQ ID NO: 916 - protein, 8 aa, Artificial Sequence
  Artificial peptide sequence
VTEKPWLI

SEQ ID NO: 917 - protein, 8 aa, Artificial Sequence
  Artificial peptide sequence
VTEKPWLV

SEQ ID NO: 918 - protein, 8 aa, Artificial Sequence
  Artificial peptide sequence
VTEKPWVL

SEQ ID NO: 919 - protein, 8 aa, Artificial Sequence
  Artificial peptide sequence
VTEKPWVI

SEQ ID NO: 920 - protein, 8 aa, Artificial Sequence
  Artificial peptide sequence
VTEKPWVV

SEQ ID NO: 921 - protein, 8 aa, Artificial Sequence
  Artificial peptide sequence
VTERPYIL

SEQ ID NO: 922 - protein, 8 aa, Artificial Sequence
  Artificial peptide sequence
VTERPYII

SEQ ID NO: 923 - protein, 8 aa, Artificial Sequence
  Artificial peptide sequence
VTERPYIV

SEQ ID NO: 924 - protein, 8 aa, Artificial Sequence
  Artificial peptide sequence
VTERPYLL

SEQ ID NO: 925 - protein, 8 aa, Artificial Sequence
  Artificial peptide sequence
VTERPYLI

SEQ ID NO: 926 - protein, 8 aa, Artificial Sequence
  Artificial peptide sequence
VTERPYLV

SEQ ID NO: 927 - protein, 8 aa, Artificial Sequence
  Artificial peptide sequence
VTERPYVL

SEQ ID NO: 928 - protein, 8 aa, Artificial Sequence
  Artificial peptide sequence
VTERPYVI

SEQ ID NO: 929 - protein, 8 aa, Artificial Sequence
  Artificial peptide sequence
VTERPYVV

SEQ ID NO: 930 - protein, 8 aa, Artificial Sequence
  Artificial peptide sequence
VTERPWIL

SEQ ID NO: 931 - protein, 8 aa, Artificial Sequence
  Artificial peptide sequence
VTERPWII

SEQ ID NO: 932 - protein, 8 aa, Artificial Sequence
  Artificial peptide sequence
VTERPWIV

SEQ ID NO: 933 - protein, 8 aa, Artificial Sequence
  Artificial peptide sequence
VTERPWLL

SEQ ID NO: 934 - protein, 8 aa, Artificial Sequence
  Artificial peptide sequence
VTERPWLI

SEQ ID NO: 935 - protein, 8 aa, Artificial Sequence
  Artificial peptide sequence
VTERPWLV

SEQ ID NO: 936 - protein, 8 aa, Artificial Sequence
  Artificial peptide sequence
VTERPWVL

SEQ ID NO: 937 - protein, 8 aa, Artificial Sequence
  Artificial peptide sequence
VTERPWVI

SEQ ID NO: 938 - protein, 8 aa, Artificial Sequence
  Artificial peptide sequence
VTERPWVV

SEQ ID NO: 939 - protein, 8 aa, Artificial Sequence
  Artificial peptide sequence
YSDKPYIL

SEQ ID NO: 940 - protein, 8 aa, Artificial Sequence
  Artificial peptide sequence
WSDKPYIL

SEQ ID NO: 941 - protein, 8 aa, Artificial Sequence
  Artificial peptide sequence
TSDKPYIL

SEQ ID NO: 942 - protein, 8 aa, Artificial Sequence
  Artificial peptide sequence
SSDKPYIL

SEQ ID NO: 943 - protein, 8 aa, Artificial Sequence
  Artificial peptide sequence
RSDKPYIL

SEQ ID NO: 944 - protein, 8 aa, Artificial Sequence
  Artificial peptide sequence
QSDKPYIL

SEQ ID NO: 945 - protein, 8 aa, Artificial Sequence
  Artificial peptide sequence
PSDKPYIL

SEQ ID NO: 946 - protein, 8 aa, Artificial Sequence
  Artificial peptide sequence
NSDKPYIL

SEQ ID NO: 947 - protein, 8 aa, Artificial Sequence
  Artificial peptide sequence
MSDKPYIL

SEQ ID NO: 948 - protein, 8 aa, Artificial Sequence
  Artificial peptide sequence
KSDKPYIL

SEQ ID NO: 949 - protein, 8 aa, Artificial Sequence
  Artificial peptide sequence
HSDKPYIL

SEQ ID NO: 950 - protein, 8 aa, Artificial Sequence
  Artificial peptide sequence
GSDKPYIL

SEQ ID NO: 951 - protein, 8 aa, Artificial Sequence
  Artificial peptide sequence
FSDKPYIL

SEQ ID NO: 952 - protein, 8 aa, Artificial Sequence
  Artificial peptide sequence
ESDKPYIL

SEQ ID NO: 953 - protein, 8 aa, Artificial Sequence
  Artificial peptide sequence
DSDKPYIL

SEQ ID NO: 954 - protein, 8 aa, Artificial Sequence
  Artificial peptide sequence
CSDKPYIL

SEQ ID NO: 955 - protein, 7 aa, Artificial Sequence
  Artificial peptide sequence
YDKPYIL

SEQ ID NO: 956 - protein, 7 aa, Artificial Sequence
  Artificial peptide sequence
WDKPYIL

SEQ ID NO: 957 - protein, 7 aa, Artificial Sequence
  Artificial peptide sequence
VDKPYIL

SEQ ID NO: 958 - protein, 7 aa, Artificial Sequence
  Artificial peptide sequence
RDKPYIL

SEQ ID NO: 959 - protein, 7 aa, Artificial Sequence
  Artificial peptide sequence
QDKPYIL

SEQ ID NO: 960 - protein, 7 aa, Artificial Sequence
  Artificial peptide sequence
PDKPYIL

SEQ ID NO: 961 - protein, 7 aa, Artificial Sequence
  Artificial peptide sequence
NDKPYIL

SEQ ID NO: 962 - protein, 7 aa, Artificial Sequence
  Artificial peptide sequence
MDKPYIL

SEQ ID NO: 963 - protein, 7 aa, Artificial Sequence
  Artificial peptide sequence
LDKPYIL

SEQ ID NO: 964 - protein, 7 aa, Artificial Sequence
  Artificial peptide sequence
KDKPYIL

SEQ ID NO: 965 - protein, 7 aa, Artificial Sequence
  Artificial peptide sequence
IDKPYIL

SEQ ID NO: 966 - protein, 7 aa, Artificial Sequence
  Artificial peptide sequence
HDKPYIL

SEQ ID NO: 967 - protein, 7 aa, Artificial Sequence
  Artificial peptide sequence
GDKPYIL

SEQ ID NO: 968 - protein, 7 aa, Artificial Sequence
  Artificial peptide sequence
FDKPYIL

SEQ ID NO: 969 - protein, 7 aa, Artificial Sequence
  Artificial peptide sequence
EDKPYIL

SEQ ID NO: 970 - protein, 7 aa, Artificial Sequence
  Artificial peptide sequence
DDKPYIL

SEQ ID NO: 971 - protein, 7 aa, Artificial Sequence
  Artificial peptide sequence
CDKPYIL

SEQ ID NO: 972 - protein, 7 aa, Artificial Sequence
  Artificial peptide sequence
ADKPYIL

SEQ ID NO: 973 - protein, 6 aa, Artificial Sequence
  Artificial peptide sequence
YKPYIL

SEQ ID NO: 974 - protein, 6 aa, Artificial Sequence
  Artificial peptide sequence
WKPYIL

SEQ ID NO: 975 - protein, 6 aa, Artificial Sequence
  Artificial peptide sequence
VKPYIL

SEQ ID NO: 976 - protein, 6 aa, Artificial Sequence
  Artificial peptide sequence
TKPYIL

SEQ ID NO: 977 - protein, 6 aa, Artificial Sequence
  Artificial peptide sequence
SKPYIL

SEQ ID NO: 978 - protein, 6 aa, Artificial Sequence
  Artificial peptide sequence
QKPYIL

SEQ ID NO: 979 - protein, 6 aa, Artificial Sequence
  Artificial peptide sequence
PKPYIL

SEQ ID NO: 980 - protein, 6 aa, Artificial Sequence
  Artificial peptide sequence
NKPYIL

SEQ ID NO: 981 - protein, 6 aa, Artificial Sequence
  Artificial peptide sequence
MKPYIL

SEQ ID NO: 982 - protein, 6 aa, Artificial Sequence
  Artificial peptide sequence
LKPYIL

SEQ ID NO: 983 - protein, 6 aa, Artificial Sequence
  Artificial peptide sequence
IKPYIL

SEQ ID NO: 984 - protein, 6 aa, Artificial Sequence
  Artificial peptide sequence
HKPYIL

SEQ ID NO: 985 - protein, 6 aa, Artificial Sequence
  Artificial peptide sequence
FKPYIL

SEQ ID NO: 986 - protein, 6 aa, Artificial Sequence
  Artificial peptide sequence
CKPYIL

SEQ ID NO: 987 - protein, 6 aa, Artificial Sequence
  Artificial peptide sequence
AKPYIL

SEQ ID NO: 988 - protein, 5 aa, Artificial Sequence
  Artificial peptide sequence
YPYIL

SEQ ID NO: 989 - protein, 5 aa, Artificial Sequence
  Artificial peptide sequence
WPYIL

SEQ ID NO: 990 - protein, 5 aa, Artificial Sequence
  Artificial peptide sequence
VPYIL

SEQ ID NO: 991 - protein, 5 aa, Artificial Sequence
  Artificial peptide sequence
TPYIL

SEQ ID NO: 992 - protein, 5 aa, Artificial Sequence
  Artificial peptide sequence
SPYIL

SEQ ID NO: 993 - protein, 5 aa, Artificial Sequence
  Artificial peptide sequence
QPYIL

SEQ ID NO: 994 - protein, 5 aa, Artificial Sequence
  Artificial peptide sequence
PPYIL

SEQ ID NO: 995 - protein, 5 aa, Artificial Sequence
  Artificial peptide sequence
NPYIL

SEQ ID NO: 996 - protein, 5 aa, Artificial Sequence
  Artificial peptide sequence
MPYIL

SEQ ID NO: 997 - protein, 5 aa, Artificial Sequence
  Artificial peptide sequence
LPYIL

SEQ ID NO: 998 - protein, 5 aa, Artificial Sequence
  Artificial peptide sequence
IPYIL

SEQ ID NO: 999 - protein, 5 aa, Artificial Sequence
  Artificial peptide sequence
HPYIL

SEQ ID NO: 1000 - protein, 5 aa, Artificial Sequence
  Artificial peptide sequence
GPYIL

SEQ ID NO: 1001 - protein, 5 aa, Artificial Sequence
  Artificial peptide sequence
FPYIL

SEQ ID NO: 1002 - protein, 5 aa, Artificial Sequence
  Artificial peptide sequence
EPYIL

SEQ ID NO: 1003 - protein, 5 aa, Artificial Sequence
  Artificial peptide sequence
DPYIL

SEQ ID NO: 1004 - protein, 5 aa, Artificial Sequence
  Artificial peptide sequence
CPYIL

SEQ ID NO: 1005 - protein, 5 aa, Artificial Sequence
  Artificial peptide sequence
APYIL

SEQ ID NO: 1006 - protein, 9 aa, Artificial Sequence
  Artificial peptide sequence
ASDKPYILA

SEQ ID NO: 1007 - protein, 10 aa, Artificial Sequence
  Artificial peptide sequence
ASDKPYILAE

SEQ ID NO: 1008 - protein, 11 aa, Artificial Sequence
  Artificial peptide sequence
ASDKPYILAEE

SEQ ID NO: 1009 - protein, 12 aa, Artificial Sequence
  Artificial peptide sequence
ASDKPYILAEEL

SEQ ID NO: 1010 - protein, 13 aa, Artificial Sequence
  Artificial peptide sequence
ASDKPYILAEELR

SEQ ID NO: 1011 - protein, 14 aa, Artificial Sequence
  Artificial peptide sequence
ASDKPYILAEELRR

SEQ ID NO: 1012 - protein, 15 aa, Artificial Sequence
  Artificial peptide sequence
ASDKPYILAEELRRE

SEQ ID NO: 1013 - protein, 16 aa, Artificial Sequence
  Artificial peptide sequence
ASDKPYILAEELRREL

SEQ ID NO: 1014 - protein, 17 aa, Artificial Sequence
  Artificial peptide sequence
ASDKPYILAEELRRELP

SEQ ID NO: 1015 - protein, 18 aa, Artificial Sequence
  Artificial peptide sequence
ASDKPYILAEELRRELPP

SEQ ID NO: 1016 - protein, 4 aa, Artificial Sequence
  Artificial peptide sequence
AEEL

SEQ ID NO: 1017 - protein, 5 aa, Artificial Sequence
  Artificial peptide sequence
AEELR

SEQ ID NO: 1018 - protein, 6 aa, Artificial Sequence
  Artificial peptide sequence
AEELRR

SEQ ID NO: 1019 - protein, 7 aa, Artificial Sequence
  Artificial peptide sequence
AEELRRE

SEQ ID NO: 1020 - protein, 8 aa, Artificial Sequence
  Artificial peptide sequence
AEELRREL

SEQ ID NO: 1021 - protein, 9 aa, Artificial Sequence
  Artificial peptide sequence
AEELRRELP

SEQ ID NO: 1022 - protein, 10 aa, Artificial Sequence
  Artificial peptide sequence
AEELRRELPP

SEQ ID NO: 1023 - protein, 7 aa, Artificial Sequence
  Artificial peptide sequence
ASDKPYI

SEQ ID NO: 1024 - protein, 6 aa, Artificial Sequence
  Artificial peptide sequence
ASDKPY

SEQ ID NO: 1025 - protein, 5 aa, Artificial Sequence
  Artificial peptide sequence
ASDKP

SEQ ID NO: 1026 - protein, 8 aa, Artificial Sequence
  Artificial peptide sequence
AADKPYIL

SEQ ID NO: 1027 - protein, 8 aa, Artificial Sequence
  Artificial peptide sequence
ASAKPYIL

SEQ ID NO: 1028 - protein, 8 aa, Artificial Sequence
  Artificial peptide sequence
ASDAPYIL

SEQ ID NO: 1029 - protein, 8 aa, Artificial Sequence
  Artificial peptide sequence
ASDKAYIL

SEQ ID NO: 1030 - protein, 8 aa, Artificial Sequence
  Artificial peptide sequence
ASDKPAIL

SEQ ID NO: 1031 - protein, 8 aa, Artificial Sequence
  Artificial peptide sequence
ASDKPYAL

SEQ ID NO: 1032 - protein, 8 aa, Artificial Sequence
  Artificial peptide sequence
ASDKPYIA

SEQ ID NO: 1033 - protein, 169 aa, Mus musculus
MRGMNLQLVCLTLLAFSSWSLCSDSEEDVRALEADLLTNMHTSKISKASPPSWKMTLLNVCSLINNVNSPAEEAGDMHDDDLVGKRKLPLVLDGFSLEAMLTIFQLQKICRSRAFQHWEIIQEDILDNVNDKNEKEEVIKRKIPYILKRQLYENKPRRPYILKRGSYYY

SEQ ID NO: 1034 - protein, 25 aa, Homo sapiens
MLTKFETKSARVKGLSFHPKRPWIL

SEQ ID NO: 1035 - protein, 64 aa, Homo sapiens
VMASFKILAGDKNYITMDELRRELPPDQAEYCIARMAPYTGPDSVPGALDYMSFSTALYGESDL

SEQ ID NO: 1036 - protein, 64 aa, Homo sapiens
VIASFKVLAGDKNFITAEELRRELPPDQAEYCIARMAPYQGPDAVPGALDYKSFSTALYGESDL

SEQ ID NO: 1037 - protein, 64 aa, Homo sapiens
VIASFRILASDKPYILAEELRRELPPDQAQYCIKRMPAYSGPGSVPGALDYAAFSSALYGESDL

SEQ ID NO: 1038 - protein, 63 aa, Homo sapiens
VVASFKILAGDKNYITPEELRRELPAKQAEYCIRRMVPYKGSGAPAGALDYVAFSSALYGESD

SEQ ID NO: 1039 - protein, 64 aa, Drosophila miranda
VIDSFRILAADKPYILPDELRRELPPDQAEYCIQRMPPYKGPNGVPGALDYMSFSTALYGETDL

SEQ ID NO: 1040 - protein, 61 aa, Caenorhabditis elegans
MIDSFRILASGKTFITADELERELPRDQAAYCMARMAPSREPGAPPRSFDYVTFSRSLYSQ

SEQ ID NO: 1041 - protein, 55 aa, Dictyostelium discoideum
TKAAFKVMAEDKDFITEAQIRAAISDSKQIDYLLASMPAVEGGFDYNSFAEKLYQ

SEQ ID NO: 1042 - protein, 61 aa, Schizosaccharomyces pombe
VLYAFCDVADGKSYVTSDDLLRSQVRPNIVKFLECNMNKHSEGLDYLTWIKQLLAEDKEIV

SEQ ID NO: 1043 - protein, 8 aa, Artificial Sequence
  Artificial peptide sequence
ASGKTFIT

SEQ ID NO: 1044 - protein, 8 aa, Artificial Sequence
  Artificial peptide sequence
ADGKSYVT

SEQ ID NO: 1045 - protein, 7 aa, Artificial Sequence
  Artificial peptide sequence
DSKPYIL

SEQ ID NO: 1046 - protein, 6 aa, Artificial Sequence
  Artificial peptide sequence
SKPYIL

(19) United States
(12) Patent Application Publication
(10) Pub. No.: US 2023/0042729 A1
(43) Pub. Date: Feb. 9, 2023

(54) PEPTIDE CONSTRUCTS AND COMPOSITIONS

(71) Applicant: DIET4LIFE APS, Hammel (DK)

(72) Inventors: Jan Stagsted, Hammel (DK); Jiehui Zhou, Brabrand (DK); Randi Jessen, Trige (DK)

(21) Appl. No.: 17/438,762

(22) PCT Filed: Mar. 13, 2020
(86) PCT No.: PCT/EP2020/056924

§ 371 Date: Sep. 13, 2021

(30) Foreign Application Priority Data
Mar. 15, 2019 (EP) 19163073.0

(51) Int. Cl. A61K 38/08; A61K 36/81; A61P 3/04; A61K 9/00

(52) U.S. Cl. CPC A61K 38/08; A61K 36/81; A61P 3/04; A61K 9/0053

(57) ABSTRACT

The present invention relates to novel peptides, composition comprising such peptides including nutritional supplements and methods for inducing satiation and satiety, for weight management and preventing or reducing the incidence of obesity, or for preventing or reducing cardiovascular diseases, atherosclerosis, hypertension, hepatosteatosis, cancer and/or diabetes.

### CROSS-REFERENCE TO RELATED APPLICATIONS

This application is a national stage filing under 35 U.S.C. 371 of PCT/EP2020/056924 filed Mar. 13, 2020, which was published by the International Bureau in English on Sep. 24, 2020, and which claims priority from European Application No. 19163073.0, filed Mar. 15, 2019, each of which is hereby incorporated in its entirety by reference in this application.

### REFERENCE TO SEQUENCE LISTING SUBMITTED ELECTRONICALLY

The official copy of the sequence listing is submitted electronically via EFS-Web as an ASCII formatted sequence listing with a file named SEQLTXT-1.txt, created on Sep. 13, 2021, and having a size of 206,172 bytes, which is identical to the sequence listing submitted for International Application No. PCT/EP2020/056924 on Mar. 13, 2020. The sequence listing contained in this ASCII formatted document is part of the specification and is herein incorporated by reference in its entirety.

### FIELD OF THE INVENTION

The present invention relates to novel constructs of peptides, composition comprising such peptide constructs including nutritional supplements and methods for inducing satiation and satiety, for preventing or reducing the incidence of metabolic syndrome comprising overweight and obesity, cardiovascular diseases, atherosclerosis, hypertension, hepatosteatosis, diabetes and/or cancer.

### BACKGROUND OF THE INVENTION

Obesity is a common medical condition affecting numerous humans throughout the world and is associated with, induces or increases the risk of developing conditions such as cardiovascular diseases, atherosclerosis, hypertension, hepatosteatosis, cancer and/or diabetes.

Some regulators of obesity have been identified. However, despite intensive study, the regulation of obesity is still poorly understood.

Protein is more satiating than carbohydrate and fat, and its effect on food intake is more than can be accounted for by its energy content alone. The mechanism by which proteins trigger food intake regulatory systems is unclear. However, it seems likely that satiety signals arising from protein ingestion begin in the gastrointestinal tract upon proteolytic digestion.

Accordingly, dietary proteolytic products (peptides and amino acids) induce signalling in enteroendocrine cells of the intestine, which leads to secretion of various gut hormones, e.g. glucagon-like peptide-1 (GLP-1) (FIG. 1) with neuronal, local (auto- and paracrine) and systemic (endocrine) effects (FIG. 2), ultimately leading to satiation (amount of food ingested as a meal) and satiety (length of time between meals). It is well-known that (some) enteroendocrine cells respond to free amino acids and small peptides (di- and tripeptides), which are readily taken up by the enterocytes and metabolized and/or transported into systemic circulation. Rate of digestion, i.e. transit time in the GI tract, secretion of digestive enzymes, etc, is a highly regulated process, where cellular responses to undigested proteins and/or increases in amino acids and peptides in the gut leads to secretion of gut hormones, e.g. GLP-1, peptide tyrosine-tyrosine (PYY), neurotensin (NT), which induces satiation. If these signals persist in the gut because of slow and prolonged release, satiety is enhanced. One such mechanism is the ileal brake, where unknown components in partly digested food reaches the distal small intestine and invokes a response in the form of secretion of the gut hormones GLP-1, PYY, NT and possibly others, as yet unknown hormones. However, the precise mechanism behind the ileal brake is unknown.

The specific peptide(s) responsible for this satiety inducing signal(s) is largely unknown and it would be of great importance if any of these peptides could be identified. Also, such peptides may have to be protected from degradation in the digestive system and methods for such protection would be a significant benefit.

### OBJECT OF THE INVENTION

It is an object of embodiments of the invention to provide new polypeptides that induce or signals satiety in a subject.

It is a further object of embodiments of the invention to provide these new peptides in a form that is protected from degradation in the digestive system. This may be accomplished either by modification of the peptides themselves, and/or by providing these peptides in composition with components that inhibit this degradation.

The polypeptides of the invention may be used to treat conditions associated with a wide variety of metabolic diseases, for use in weight management, and/or for preventing or reducing the incidence of overweight and/or obesity, or for preventing or reducing cardiovascular diseases, atherosclerosis, hypertension, hepatosteatosis, cancer and/or diabetes.

### SUMMARY OF THE INVENTION

Dietary proteolytic products (peptides and amino acids) induce signalling in enteroendocrine cells of the intestine, which leads to secretion of various gut hormones, e.g. GLP-1 (FIG. 1) with both central (CNS), local (auto- and paracrine) and systemic (endocrine) effects (FIG. 2), ultimately leading to satiation and satiety.

It has been found by the present inventor(s) that novel meat-derived polypeptides are superior in signalling of intestinal cell lines (FIG. 3) and that only very specific peptides are capable of signalling (FIG. 4). The inventors of the present invention have identified polypeptides including an octapeptide (ASDKPYIL) present in proteolytic digests (FIG. 5) and resistant to pepsin degradation, of which a pentapeptide (KPYIL) is the minimal sequence with significant biologic activity (FIG. 6). The octapeptide sequence is unique for the muscle-specific alpha-actinin-2 protein, and the sequence is conserved between all animal species. This peptide would be applicable as a novel, but natural nutritional supplement to induce satiation and satiety.

The present inventors have found from stability studies of the octapeptide DC7-2 (ASDKPYIL) and truncated versions of this sequence that the peptide—not surprisingly—is degraded over time by both exo- and endopeptidases naturally present in the intestine. The peptide is resilient to degradation by gastric pepsin and gastric acidity, but sensitive to trypsin and chymotrypsin released into the intestine by the exocrine pancreas, as well as—particularly—carboxypeptidases, of which two types are also released by the pancreas: Carboxypeptidase A and B.

Action of trypsin and chymotrypsin at the lysine residue (ASDK PYIL) completely abolishes activity, as does C-terminal removal of one or more residues (ASDKPYI L).

Aminopeptidases are abundant in the brush-border of the intestine and degrade DC7-2 from the amino terminus, but because activity of the octapeptide is preserved in the hexapeptide DKPYIL, which is truncated by two amino acids from the N-terminus, partial protection is provided by AS in the DC7-2 sequence.

So, in a first aspect the present invention relates to an isolated polypeptide comprising the amino acid sequence

(formula I, SEQ ID NO: 1)
AA1-AA2-AA3-AA4-AA5-AA6-AA7-AA8-E*,

wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; wherein E* is C-terminal extension with 1-10 of any amino acids; which polypeptide is not more than 50 amino acids in length; or a variant thereof with a sequence identity of at least 80%.

In a second aspect the present invention relates to an isolated polypeptide consisting of the amino acid sequence

(formula II, SEQ ID NO: 2)
R1-AA1-AA2-AA3-AA4-AA5-AA6-AA7-AA8-E*-R2,

wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; R1 defines the N-term (—NH2) or a protection group; wherein E* is C-terminal extension with 1-10 of any amino acids; R2 defines the C-term (—COOH).

In a further aspect the present invention relates to a polypeptide having or comprising a sequence selected from ASDKPYILA (SEQ ID NO:1006), ASDKPYILAE (SEQ ID NO:1007), ASDKPYILAEE (SEQ ID NO:1008), ASDKPYILAEEL (SEQ ID NO:1009), ASDKPYILAEELR (SEQ ID NO:1010), ASDKPYILAEELRR (SEQ ID NO:1011), ASDKPYILAEELRRE (SEQ ID NO:1012), ASDKPYILAEELRREL (SEQ ID NO:1013), ASDKPYILAEELRRELP (SEQ ID NO:1014), and ASDKPYILAEELRRELPP (SEQ ID NO:1015).

In a further aspect the present invention relates to a composition comprising i) a gastrointestinal peptide hormone and ii) a protease inhibitor, such as potato proteinase inhibitor II (PI2), such as PI2 derived from a potato protein extract. In some embodiments such peptide hormone may be selected from the list consisting of Cholecystokinin (CCK), Gastrin, Secretin, Vasoactive Intestinal Peptide (VIP), Glucose-dependent insulinotropic peptide (GIP), Glucagon-like Peptide 1 and 2 (GLP-1 and -2), Bombesin, Chromogranin A, Glucagon, Insulin, Leptin, Neuropeptide Y, Neurotensin, Neuromedin, Pancreatic Polypeptide, PYY, Amylin, Oxyntomodulin, Xexin, Motilin, Grehlin, and Somatostatin, and bioactive analogues or variants of any one of these peptide hormones.

In a further aspect the present invention relates to a composition comprising i) a polypeptide comprising the amino acid sequence

(formula III, SEQ ID NO: 3)
AA1-AA2-AA3-AA4-AA5-AA6-AA7-AA8,

wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%; and ii) a protease inhibitor, such as potato proteinase inhibitor II (PI2), such as PI2 derived from a potato protein extract. In some embodiments this polypeptide may be any one described herein.

In a further aspect the present invention relates to a composition comprising a polypeptide of the invention.

For use in compositions according to the present invention, may be used a polypeptide comprising the amino acid sequence

(formula III, SEQ ID NO: 3)
AA1-AA2-AA3-AA4-AA5-AA6-AA7-AA8,

wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%.

Alternatively for use in compositions according to the present invention, may be used a polypeptide consisting of the amino acid sequence

(formula IV, SEQ ID NO: 4)
R1-AA1-AA2-AA3-AA4-AA5-AA6-AA7-AA8-R2,

wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; R1 defines the N-term (—NH2) or a protection group; R2 defines the C-term (—COOH).

Specifically for use in compositions according to the present invention, may be used a polypeptide having or comprising a sequence selected from ASDKPYIL (SEQ ID NO:6), SDKPYIL (SEQ ID NO:7), DKPYIL (SEQ ID NO:8), KPYIL (SEQ ID NO:9), AGDKNYIL (SEQ ID NO:10), AGDKNYIT (SEQ ID NO:11), AGDKSYIT (SEQ ID NO:12), ADGKPYIV (SEQ ID NO:13), AEDKDFIT (SEQ ID NO:14), AADKPYIL (SEQ ID NO:15), ATDKPYIL (SEQ ID NO:16), AGDKPYIT (SEQ ID NO: 17), ASEKPYIL (SEQ ID NO: 18), ADGKPYVT (SEQ ID NO:19), AGDKPYIL (SEQ ID NO:20), ASDKPNIL (SEQ ID NO:21), ASDKPYIT (SEQ ID NO:22), AADKPFIL (SEQ ID NO:23), ASDKAYIT (SEQ ID NO:24), AGDKAYIT (SEQ ID NO:25), ANGKPFIT (SEQ ID NO:26), AGDKNFIT (SEQ ID NO:27), ASDKSYIT (SEQ ID NO:28), ASDKTYIT (SEQ ID NO:29), ASDKNYIT (SEQ ID NO:30), AGDKKYIT (SEQ ID NO:31), AGDKNYIS (SEQ ID NO:32), AADKNYIT (SEQ ID NO:33), AGDKNYIM (SEQ ID NO:34), AADKNFIM (SEQ ID NO:35), AADKNFIT (SEQ ID NO:36), and AGDKGIRS (SEQ ID NO:37).

In a further aspect the present invention relates to a polypeptide or composition according to the invention for use in promoting satiety or for reducing feed intake in a subject, for use in weight management, and/or for preventing or reducing the incidence of overweight and/or obesity in a subject, or for preventing or reducing cardiovascular diseases, atherosclerosis, hypertension, hepatosteatosis, cancer and/or diabetes.

In a further aspect the present invention relates to a method of preventing or reducing the incidence of obesity in a subject, and/or of promoting satiety or for reducing feed intake in a subject, and/or to reduce or treat cardiovascular diseases, atherosclerosis, hypertension, hepatosteatosis, cancer and/or diabetes comprising enteral administering to a subject in need thereof a polypeptide or composition according to the present invention.

### LEGENDS TO THE FIGURES

FIG. 1. Dose-response curve for effect of protein hydrolysate on release of GLP-1 from GLUTag cells (open circles) or a control cell line (closed symbols) that does not produce GLP-1. Cells (˜5×10\hat{ }5 per sample) were incubated for up to 90 min in Dulbeccos Modified Eagle Medium (DMEM) containing 5.56 mM glucose in absence or presence of different amounts (weight/volume) of meat protein hydrolysate. Supernatant was filtered through 0.45 micron filters and assayed for content of GLP-1 as described in ELISA protocol. Data are mean+SEM from quadruplicate samples.

FIG. 2. Signaling by dietary nutrients in enteroendocrine cells. Illustration from Horm Res Paediatr. 2015; 83(1):1-10.

FIG. 3. Stimulation of cell signaling (measured as increase in intracellular fluorescence) by meat protein hydrolysates (filled symbols) or whey protein hydrolysates (open symbols) in three different intestinal cell lines: Top) a murine intestinal cell line; middle) GLUTag cells; bottom) CaCo2 cells.

FIG. 4. Size exclusion fractionation of protein hydrolysate and test of biologic activity. Absorbance at 280 nm shown by thick, solid line, activity of fractions by filled circles.

FIG. 5. Verification of identified sequence ASDKPYIL by synthetic peptide. Comparison of dose-response relationship of meat hydrolysate and pure, synthetic peptide identified by sequencing of purified fractions.

FIG. 6. Identification of minimal active sequence in ASDKPYIL in murine (mIC) and human (hIC) intestinal cells.

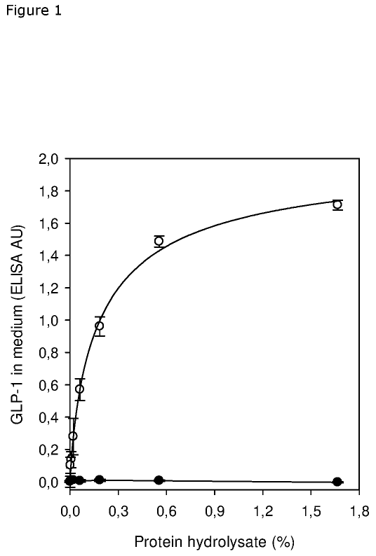

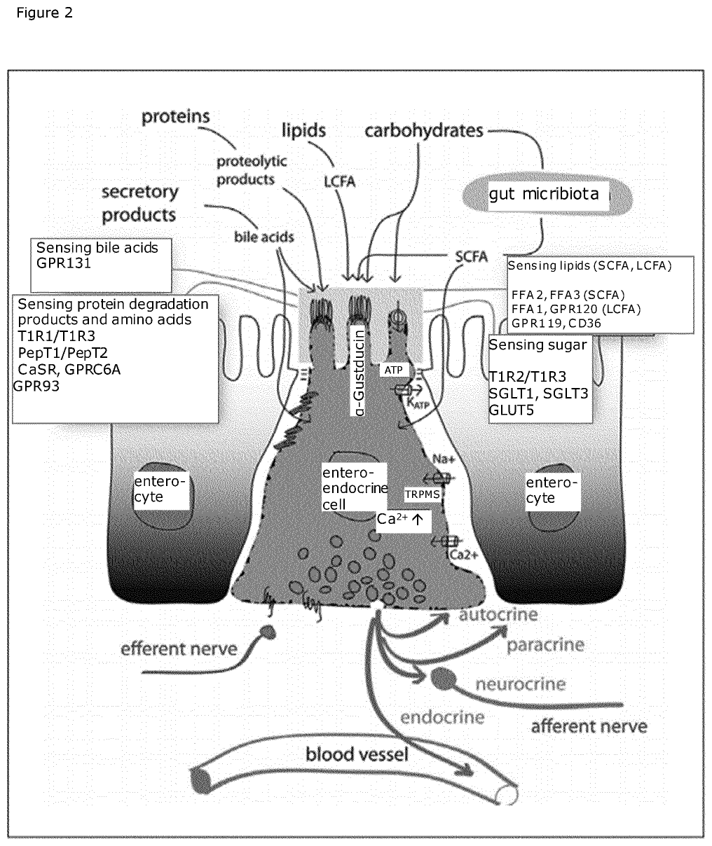

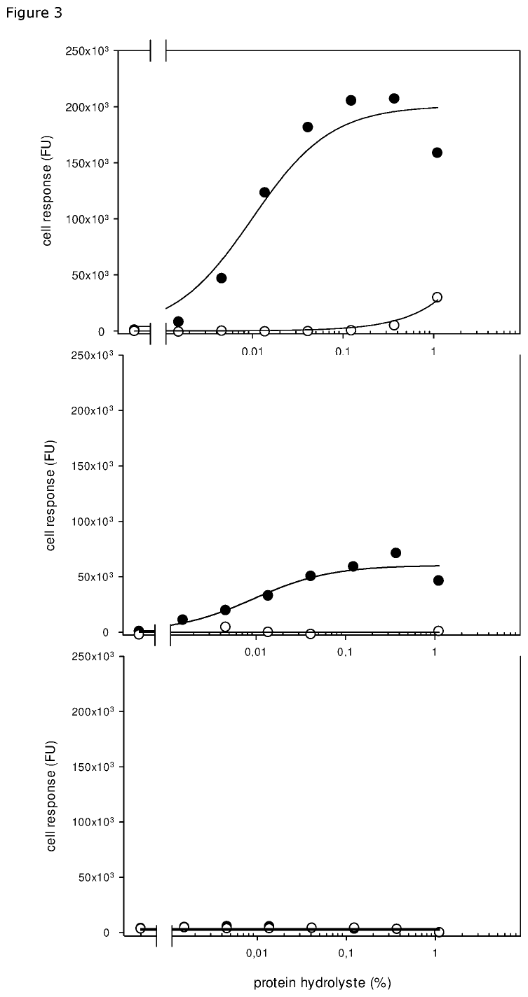

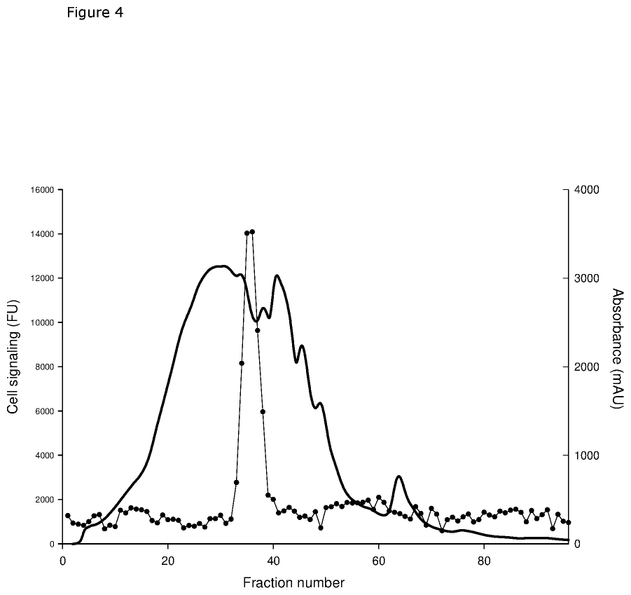

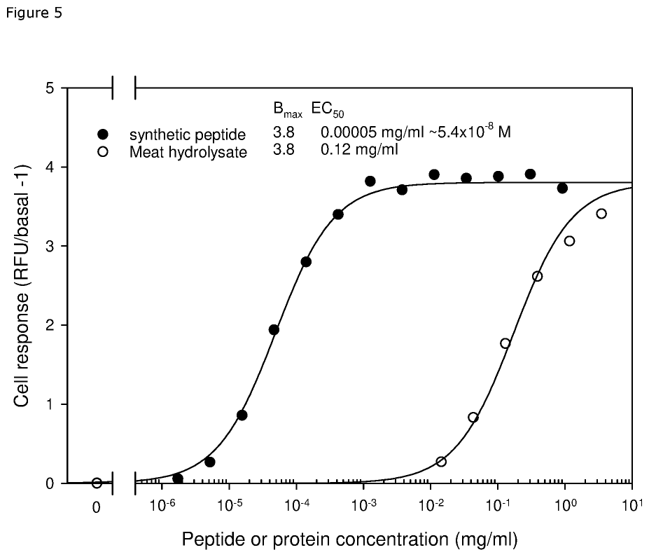

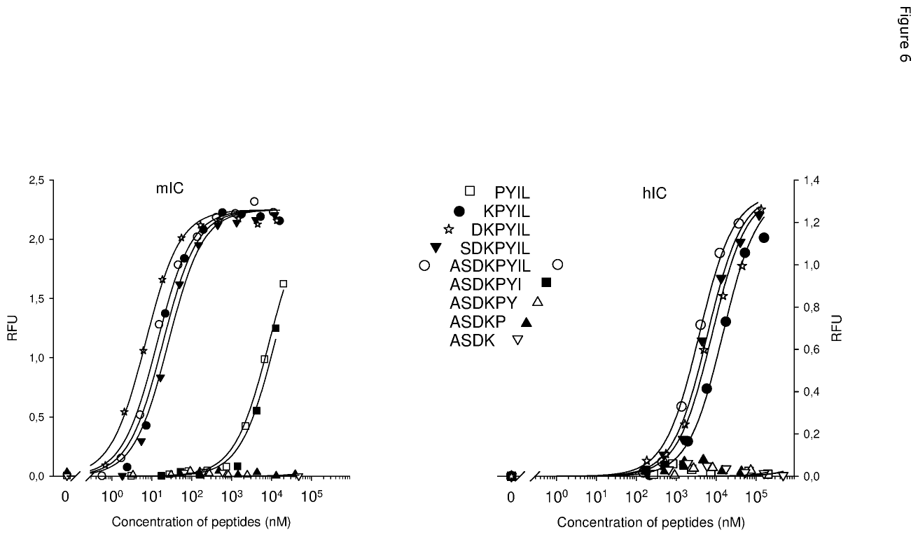

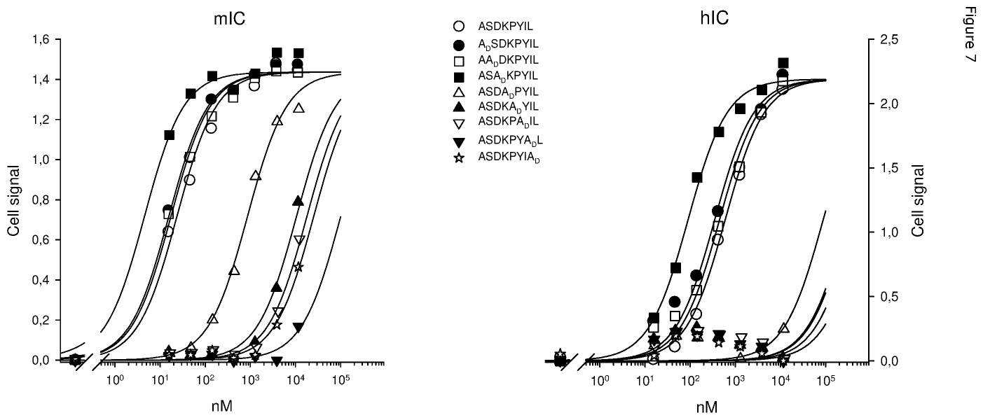

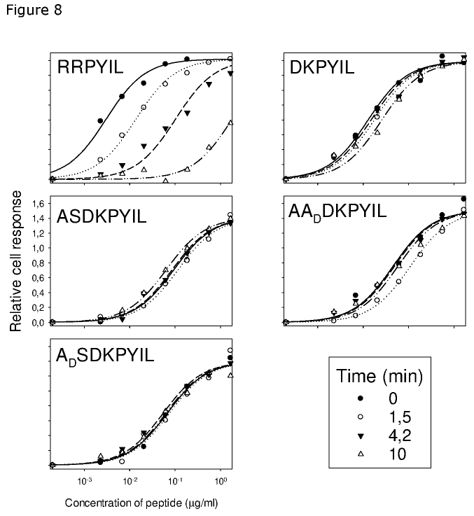

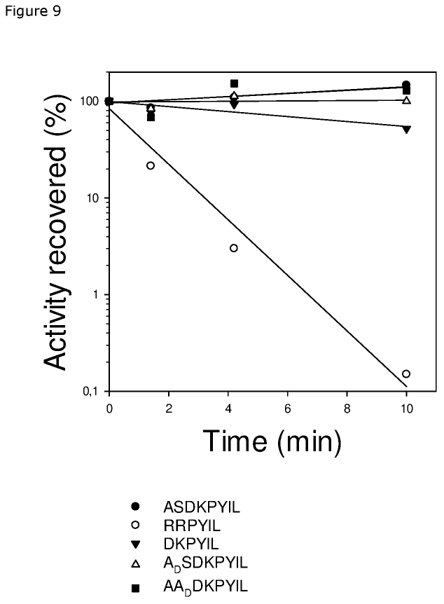

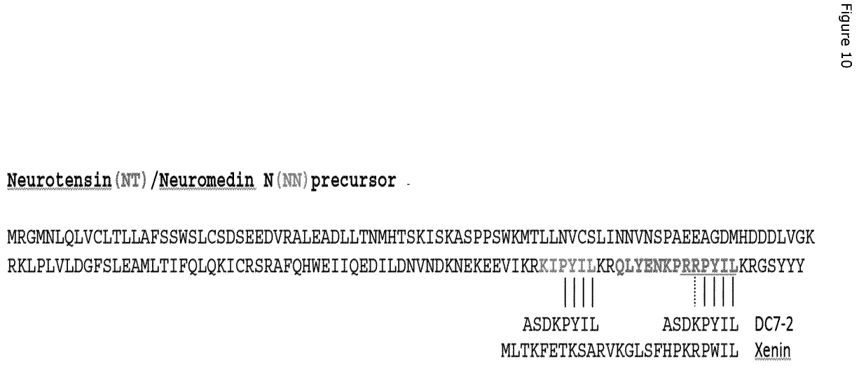

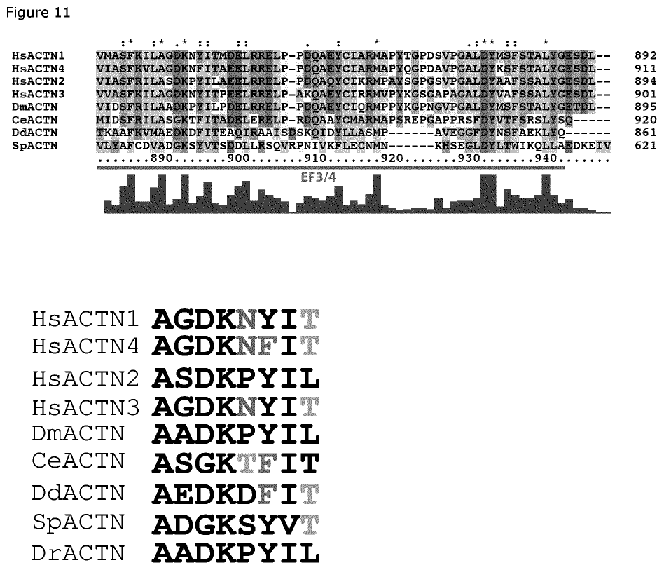

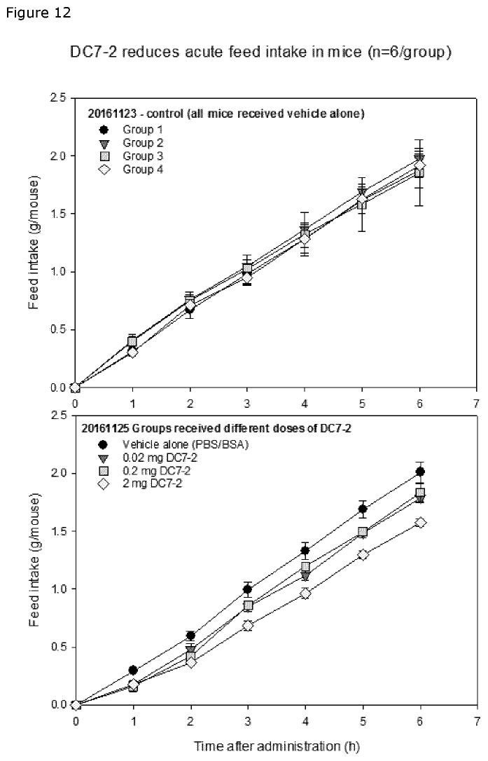

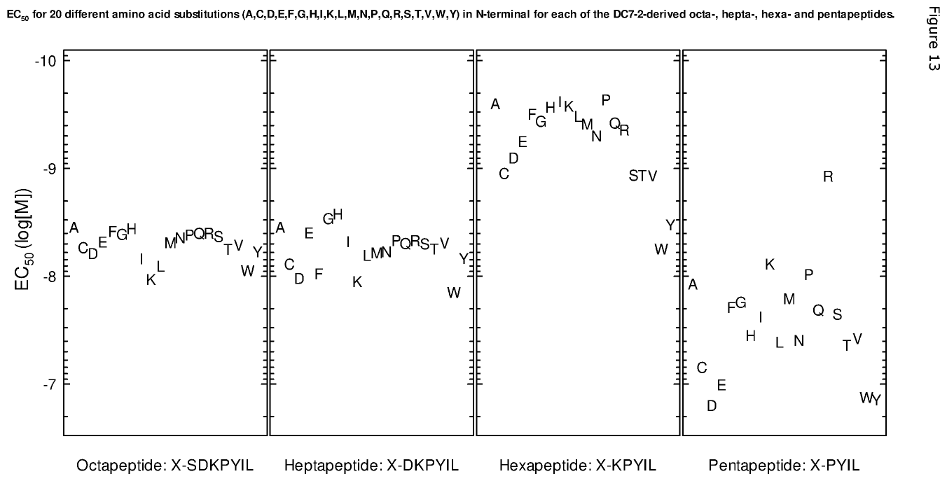

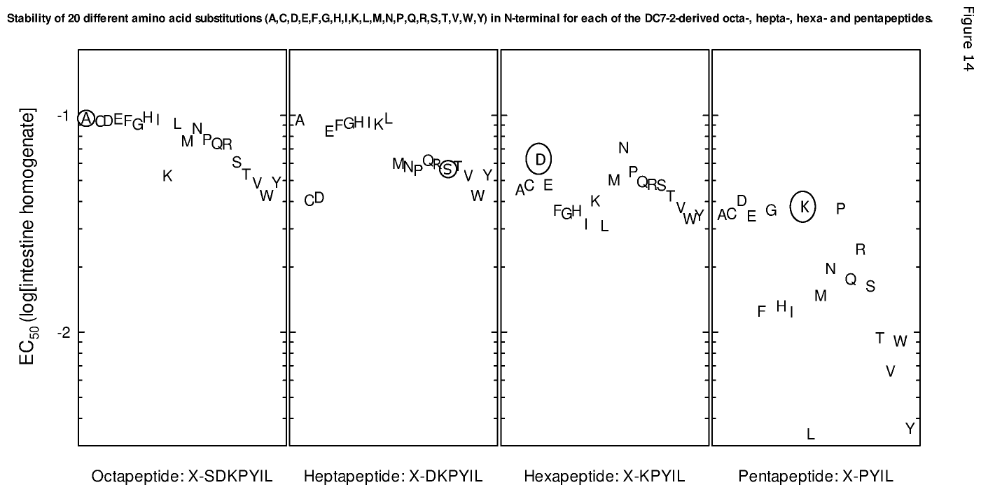

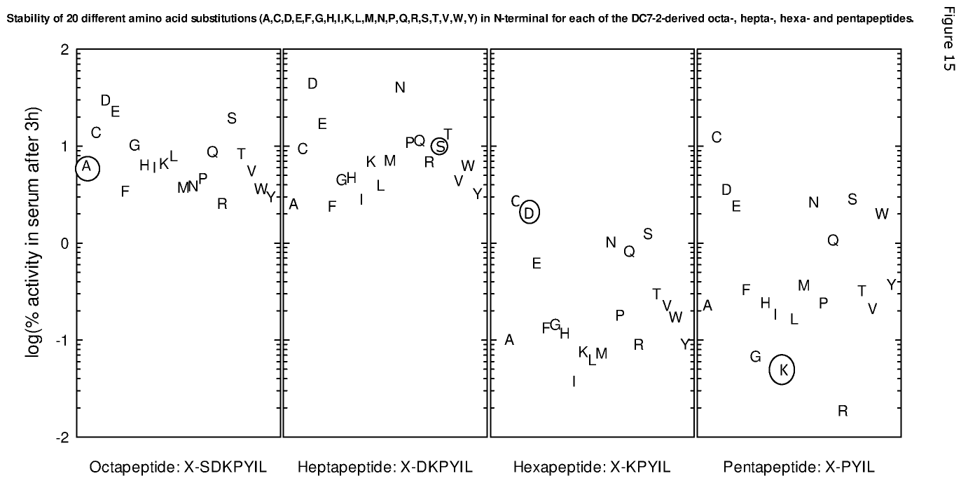

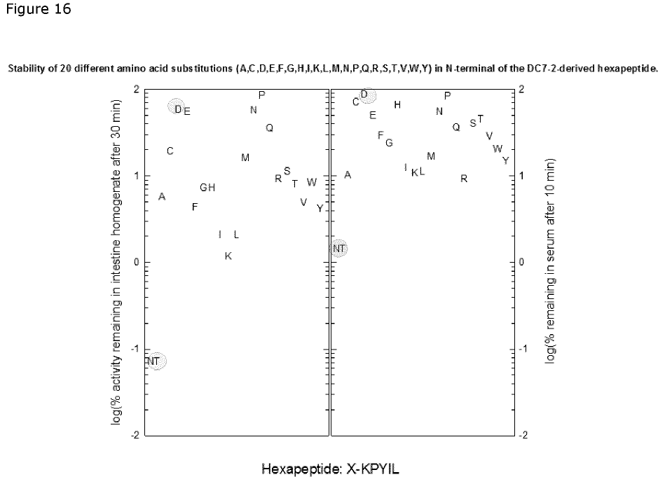

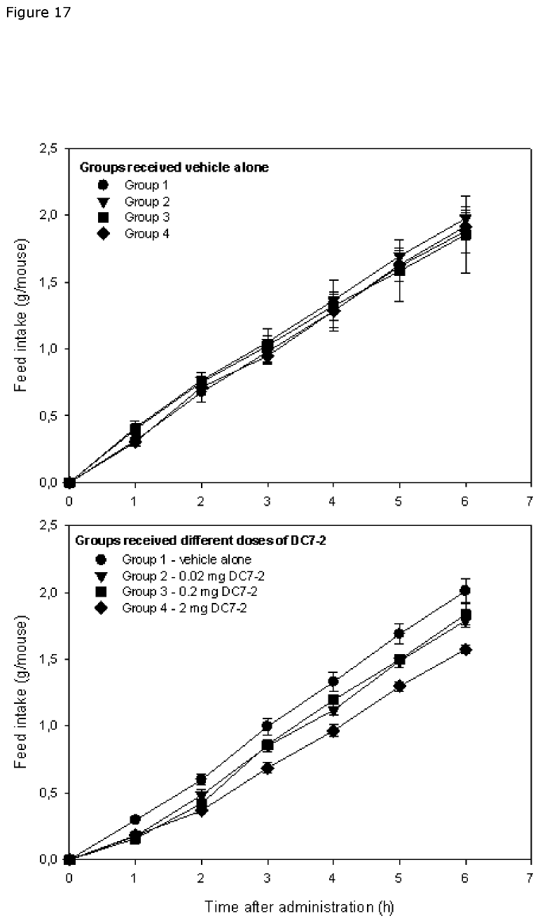

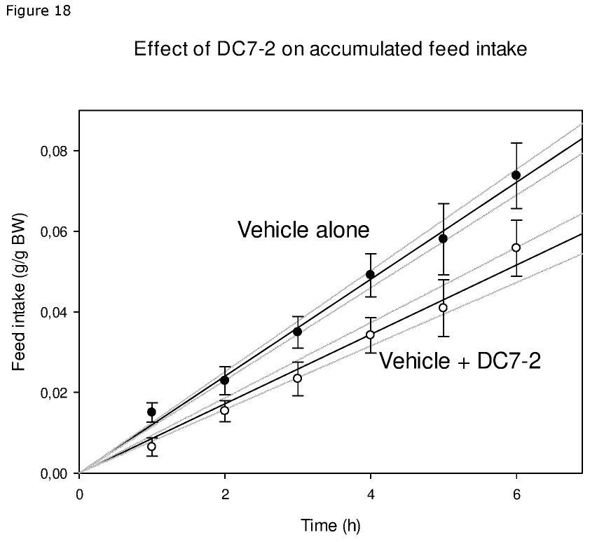

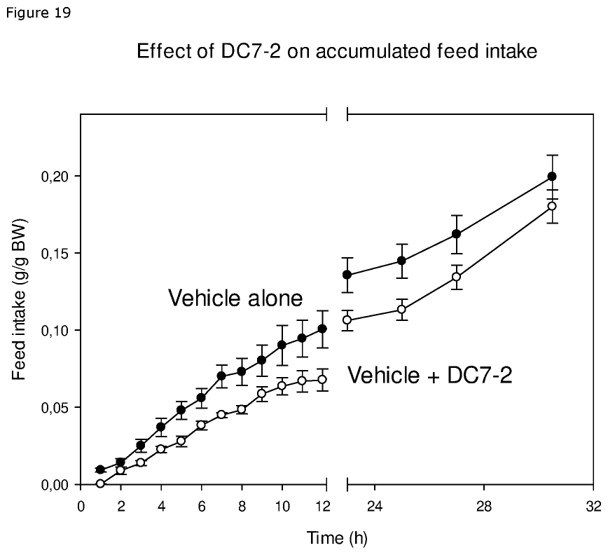

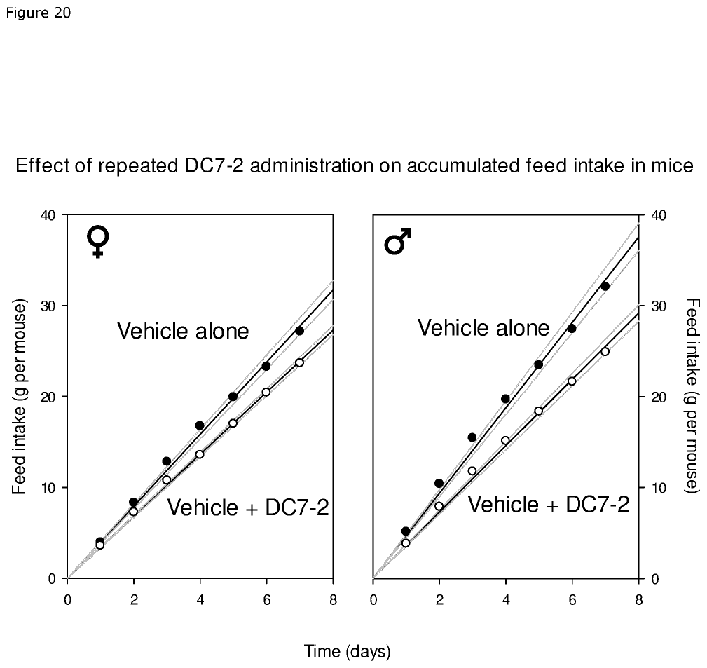

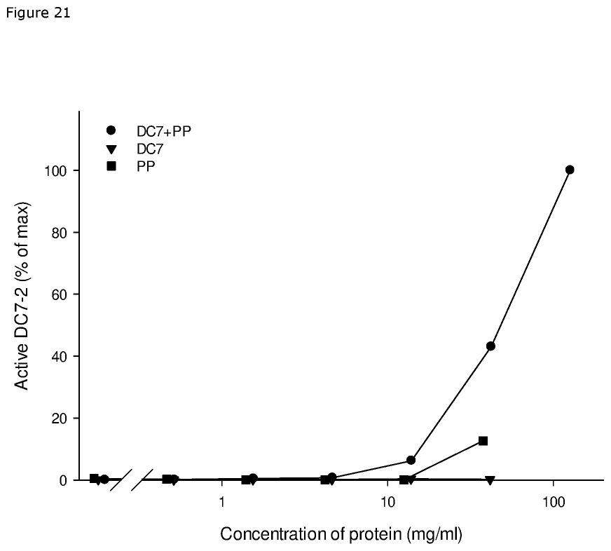

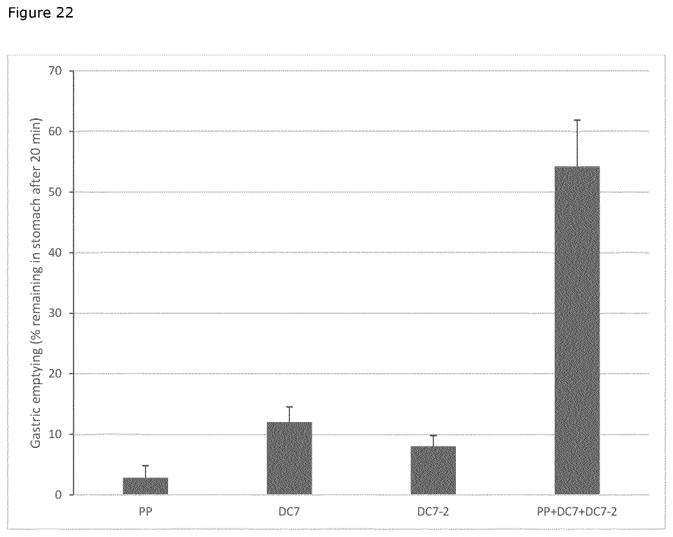

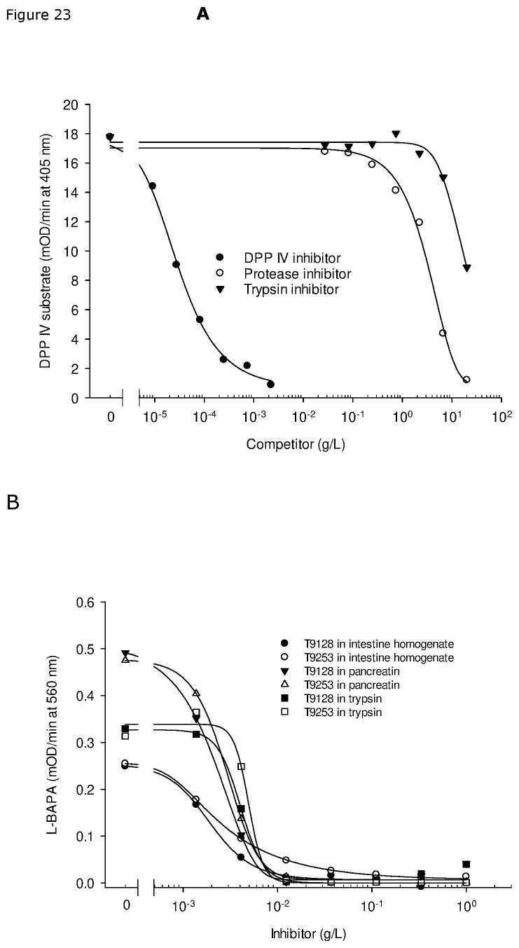

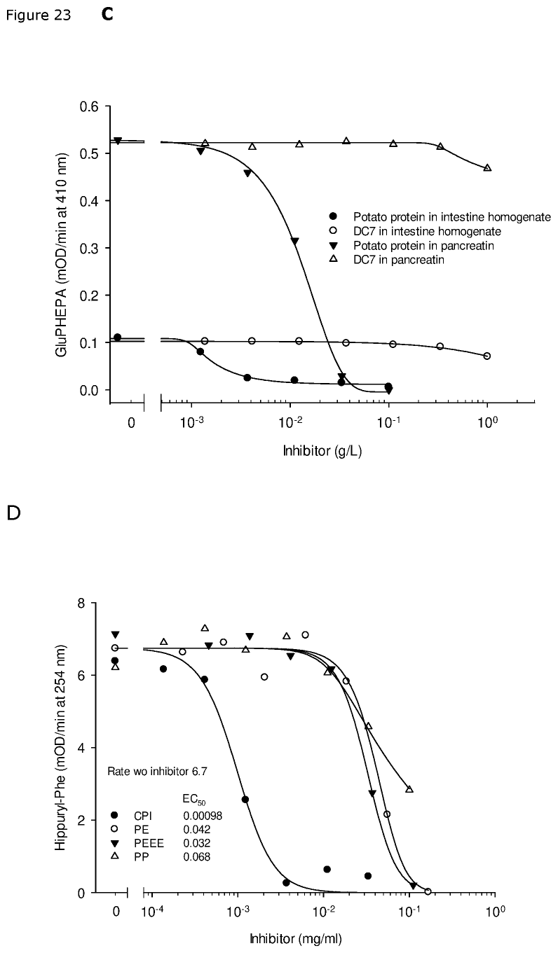

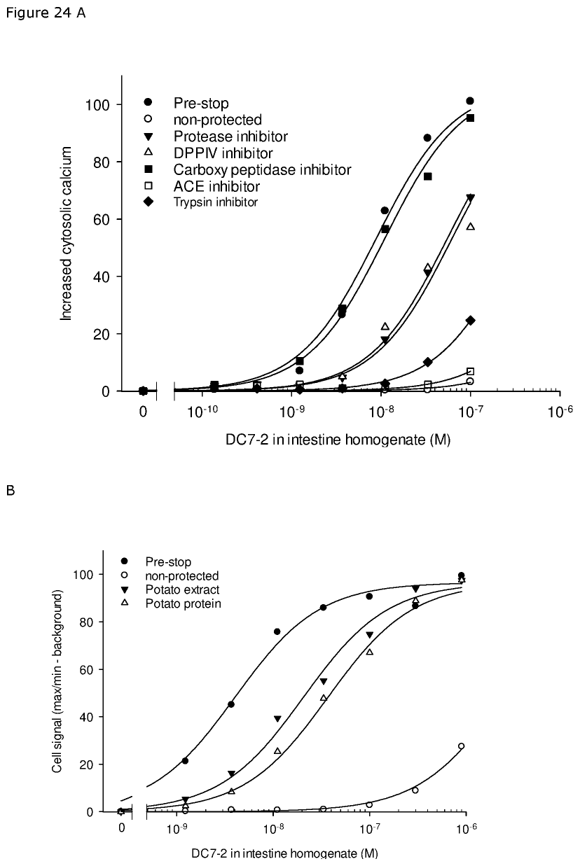

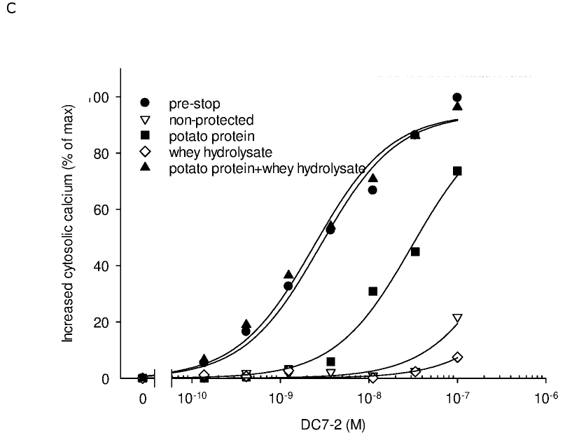

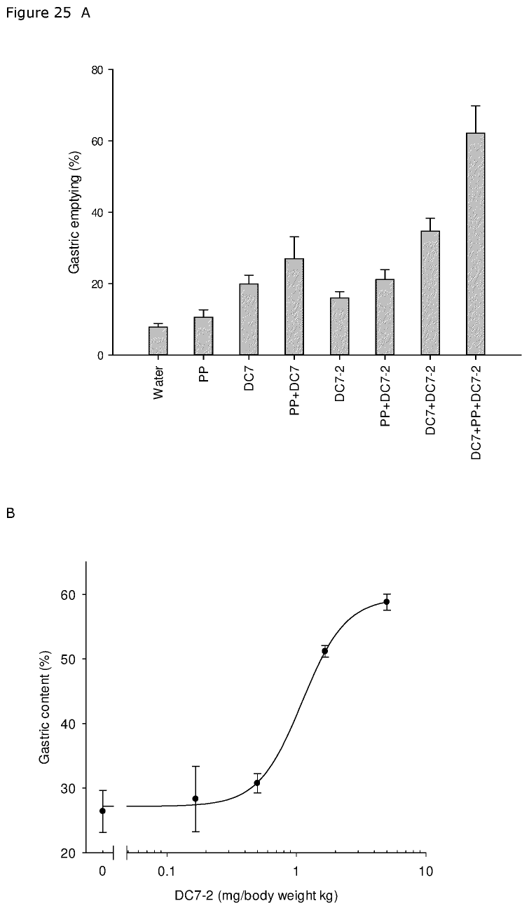

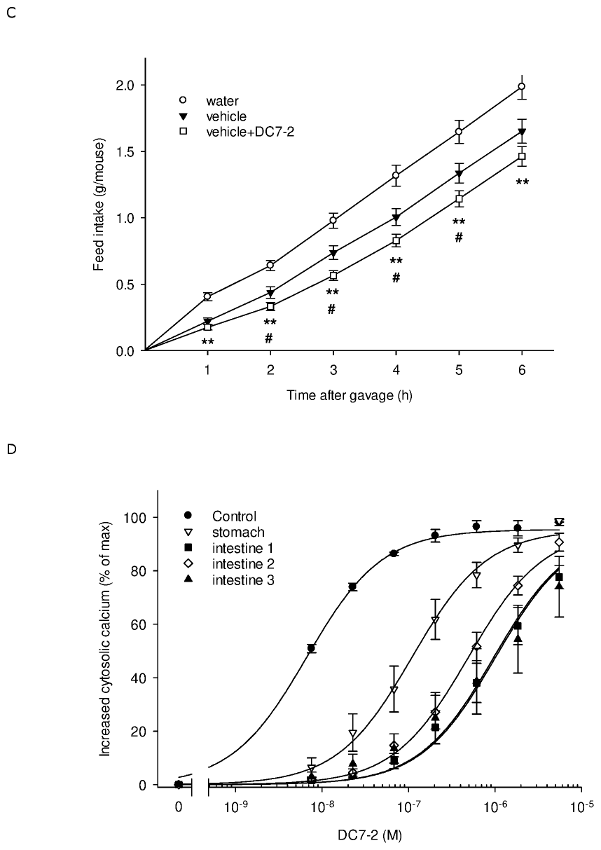

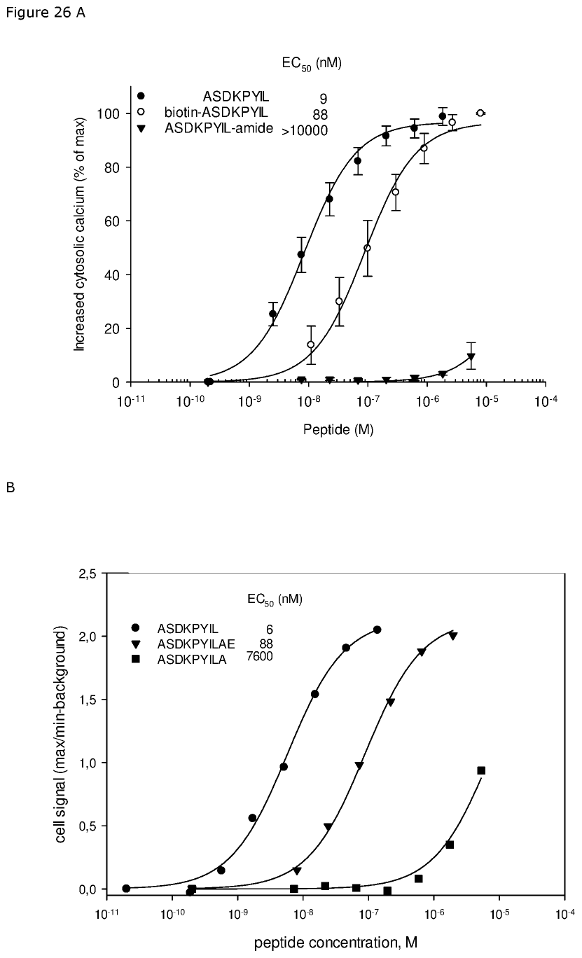

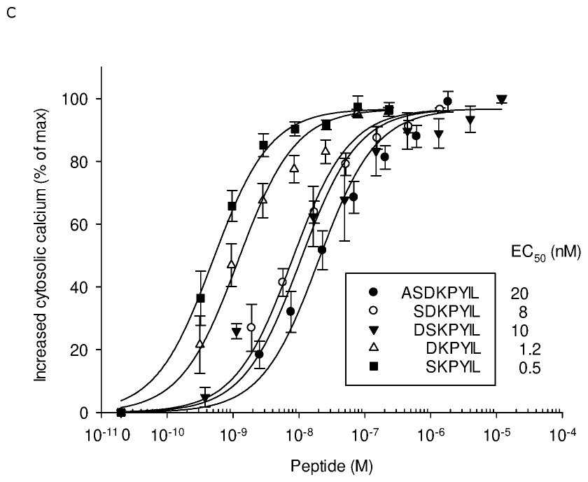

Truncation from the amino-terminal or from the carboxy-terminal end of ASDKPYIL has different consequences. Deleting the carboxy-terminal leucine reduces potency more than two orders of magnitude in mIC cells and abrogates activity in hIC. Peptides with further deletions of 2, 3 or 4 amino acids from the carboxy-terminus are without activity. Deleting the first three amino acids from the amino-terminus has no big impact on activity. However, the fourth amino acid, lysine, is critical, since PYIL has two orders of magnitude lower activity compared with the full sequence in mIC and no activity in hIC.

FIG. 7. Identification of critical residues in ASDKPYIL (d-Ala (A_{D}) scan). Systematic replacement of all residues in ASDKPYIL with the d-isomer of alanine and corresponding biological activity. Results show that 1) the last four amino acids (PYIL) are critical, 2) replacing K reduces potency more than 30-fold, 3) replacing the aspartic residue improves potency almost 10-fold, and 4) alanine and serine on the first two positions are without importance.

FIG. 8. Stability of peptides in rodent intestine.

0,001 mg/ml of the indicated peptides were incubated with pieces of rodent intestine (mouse and rat intestine gave similar results) for up to 10 minutes at 37° C. Recovery of activity was tested with dose-response curves as indicated.

FIG. 9. Stability of peptides in rodent intestine. EC₅₀ values for different peptides and different incubation times were calculated from FIG. 8 and recovered activity plotted as a function of time.

FIG. 10. Comparison of the sequences of three known gut hormones, neurotensin, neuromedin N and xenin with that of DC7-2 (ASDKPYIL). The PYIL sequence is conserved, although Y is replaced by W in xenin.

FIG. 11. Comparison of the DC7-2 sequence (aa 891-898) in isoforms of a-actinin 2 (Hs: Homo sapiens ACTN1-4) and conservation between species (Dm: Drosophila melanogaster; Ce: Caenorhabditis elegans; Dd: Dictyostelium discoideum; Sp: Schizosaccharomyces pombe; Dr: Danio rerio)

FIG. 12. 24 Balb/c female mice, 10-11 weeks, 20-22 g, were acclimatized to 12 h dark light cycle and placed single-housed in metabolic cages. Following administration of the indicated doses of DC7-2, feed and water intake was monitored for 6 h.

FIG. 13. Summary of cell signaling activities of N-terminal substitutions in octa-, hepta-, hexa- and pentapeptides based on the sequence of DC7-2. Single-letter abbreviations for the 20 amino acids are shown on the plot centered at the corresponding EC50. The native amino acid in DC7-2 is marked with a grey circle for each of the peptides.

FIG. 14. Stability of DC7-2 families of peptides in intestine homogenates. Single-letter abbreviations for the 20 amino acids are shown on the plot with the corresponding stability expressed as the logarithm to the concentration of intestine homogenate that degrades half of the activity of peptide. All peptides were incubated at 10-5 M with various dilutions of a homogenate of the entire small intestine (pool from 20 mice). After incubation for 90 min at 37° C., degradation was stopped by addition of 1 M phosphoric acid (final 0.4 M, pH˜1.2). Each peptide incubation mix was neutralized with NaOH and immediately tested for activity in intestinal cells. Control for zero degradation, i.e. addition of phosphoric acid before addition of intestine homogenate, was included for each peptide. The native amino acid in DC7-2 is marked with a grey circle for each of the peptides.

FIG. 15. Stability of DC7-2 families of peptides in serum.

FIG. 16. Stability of X-KPYIL hexapeptides in intestine homogenate and serum.

FIG. 17. 24 Balb/c female mice, 10-11 weeks, 20-22 g, were acclimatized to 12 h dark light cycle. Mice were divided into four groups each of six mice and placed single-housed in metabolic cages. Mice were then administered vehicle alone (day 1) for monitoring of feed and water intake for 6 h. On day 3, the same groups received the indicated doses of DC7-2, and feed and water intake was monitored for 6 h.

FIG. 18. Swiss Webster male mice, 25-30 g, were acclimatized to 12 h dark/light cycle and placed single-housed in cages. Following administration just prior to onset of dark cycle of vehicle alone (0.5 ml of PBS w 1% of BSA) or vehicle+DC7-2, feed intake was monitored every hour for 6 h (during dark cycle). Mean and SEM from four experiments, each with 6-8 mice per treatment. Data were fitted with linear regression (R2>0.99) and 95% confidence intervals are shown as grey lines. Accumulated feed intake for treatment with DC7-2 was 64%+/−5% compared with control for these four experiments.

FIG. 19. Swiss Webster male mice, 25-30 g, were acclimatized to 12 h dark/light cycle and placed single-housed in cages. Following administration just prior to onset of dark cycle of vehicle alone (0.5 ml of PBS w 1% of BSA) or vehicle+DC7-2, feed intake was monitored every hour for 12 h (during dark cycle) and then intermittently up to 30 h.

FIG. 20. Swiss Webster male (25-30 g) or female (20-25 g) mice were acclimatized to 12 h dark/light cycle and placed in groups of 6-8 mice per cage. Vehicle (0.5 ml of PBS w 1% of BSA) alone or vehicle+DC7-2 was administered three times per day (08:00; 16:00; 24:00), and feed intake was monitored daily for a week. Data were fitted with linear regression (R2>0.99) and 95% confidence intervals (grey lines).

FIG. 21. Protease activity in the small intestine and pancreas modelled in vitro by combining a homogenate of mouse intestine (5 mg of protein per ml) and pancreatin (6.7 mg of protein per ml, Sigma P7545 from porcine intestine) and incubating with DC7-2 in absence or presence of i) potato protein isolate (PPI) obtained by filtration from commercial production of starch and ii) crude protein hydrolysates.

FIG. 22. DC7-2 inhibits gastric emptying in mice, but only in combination with potato protein (PP) and DC7 crude protein hydrolysate.

FIG. 23. Enzymes in intestine homogenate and pancreatin. Dose-response curves of enzyme specific substrates mixed with intestine homogenate, solutions of pancreatin or enzymes with or without enzyme inhibitors (A) 1 mg/ml of the chromogenic DPP IV substrate Gly-Pro p-nitroanilide was used to measure DPP IV activity in 0.67 mg protein/ml of intestine homogenate with or without different concentrations of DPP IV inhibitor, Sitagliptin or protease inhibitor cocktail, SIGMAFAST or trypsin inhibitor, T9128. (B) Trypsin activity in 0.67 mg/ml intestine homogenate or in a solution of 0.1 mg/ml pancreatin was determined by incubation with 0.05 mg/ml Na-Benzoyl-L-arginine 4-nitroanilide hydrochloride (L-BAPA) with or without different concentrations of trypsin inhibitor, T9128 from soybean, T9253 from chicken egg white or a solution of potato protein. (C) Glutaryl-L-phenylalanine-4-nitroanilide (GluPHEPA) was used as a chymotrypsin substrate to determine activity in 0.17 mg protein/mL intestine homogenate and in 0.1 mg/ml pancreatin solution with different concentrations of trypsin inhibitor, T9128 or potato protein solution. (D) 0.5 mM Hippuryl-L-phenylalanine was used to determine carboxypeptidase activity in a 0.1 mg/mi solution of pancreatin using different concentrations of specific commercial carboxypeptidase inhibitor (CPI) or solutions of potato protein. Enzyme activities were calculated as the slope of kinetic absorbance and plotted against different concentrations of inhibitor on the x-axis. One representative experiment with each enzyme specific substrate is shown.

FIG. 24. DC7-2 is protected from degradation by proteolytic enzymes in intestine homogenate by inhibitors. Dose-response curves of mICcl2 cells stimulated by DC7-2 protected from degradation by intestinal proteolytic activity. 3 μM DC7-2 mixed with by enzyme inhibitors (SigmaFAST protease inhibitor, sitagliptin (DPP IV inhibitor), 2.5 mg/ml carboxypeptidase inhibitor, 25 mM captopril (ACE inhibitor) and 90 mg/ml solution of potato protein) was incubated with (A) 0.05 mg protein/ml intestine homogenate at 37° C. for 90 min or (B) 3.2 mg protein/ml intestine homogenate at 37° C. for 60 min. (C) 4.5 μM DC7-2 mixed with either a 10% (w/v) solution of potato protein, a 40% (w/v) solution of hydrolyzed whey protein or a mixture of 10% (w/v) potato protein and 40% (w/v) whey hydrolysate was incubated with 3.2 mg protein/ml intestine homogenate at 37° C. for 60 min.

FIG. 25. Protected DC7-2 dose-dependently delays gastric emptying and reduces feed intake in mice (A) Oral administration of DC7-2 mixed with 10% potato protein and 40% hydrolyzed bovine heart protein (DC7) delays gastric emptying. Mice were gavaged with 500 μl of deionized water (n=30), 10% (w/v) potato protein (PP) solution (n=18), 40% (w/v) DC7 solution (n=10), 10% PP mixed with 40% DC7 (n=13), 5 mg DC7-2 in water (n=23), 5 mg DC7-2 in 10% PP (n=11), 5 mg DC7-2 in 40% DC7 (n=10) or 5 mg DC7-2 in 10% PP and 40% DC7 (n=11). Content of phenol red in the stomach was measured (20 min+20 min) after oral administration of DC7-2.

(B) Oral administration of different doses of protected DC7-2 (0, 0.17, 0.5, 1.67 and 5 mg DC7-2 in 500 μl of protection vehicle containing 10% PP and 40% DC7) dose-dependently delays gastric emptying in mice (n=7).

(C) Accumulative feed intake after oral administration of water, protection vehicle (10% PP+40% DC7) or 5 mg DC7-2 in protection vehicle. Significant differences in feed intake by ANOVA are indicated by * (p<0.05) or ** (p<0.01). Values are means±SEM (n=24 per group).

(D) Dose response curves of mICcl2 cells stimulated by recovered DC7-2 activity in stomach and intestine after oral administration. Four mice were gavaved with 5 mg DC7-2 in 500 μl of protection vehicle containing 10% PP and 40% DC7. At t=50 min after treatment, stomach and intestine divided into three equally sized parts were homogenized in 5 ml of phosphoric acid at 0.4 M to stop enzymatic activity. Samples were neutralized before immediately determination of EC₅₀ values in calcium mobilization assay. For comparison, initial DC7-2 activity in the gavage mixture was measured in parallel.

FIG. 26. C-terminal modifications of DC7-2 (A) The bioactive peptide DC7-2 (ASDKPYIL) was modified with biotin attached in the N-terminus or amidated at the C-terminus or

(B) extended with one or two extra amino acids based on the sequence of α-actinin-2 on the C-terminus. (C) Swapping d and S in the heptapeptide or comparing D and S in the hexapeptide. All synthetic peptides were dissolved in deionized water at the concentration of 1 mM and tested on Fluo-4 loaded mICcl2 cells. The potencies of these peptide were compared as EC₅₀ values calculated from dose response curves. Values are mean±SEM of 3 experiments.

### DETAILED DISCLOSURE OF THE INVENTION

The inventors of the present invention have found novel polypeptides that may be used to induce signalling in intestinal cells and may consequently induce satiety. Although a specific peptide has been identified from a proteolytic digest of muscle-specific alpha-actinin-2 protein, it is envisioned that similar polypeptides will bind the same receptors in the intestine and provide the same biological activity, i.e. signal to induce satiation and satiety. Similar peptides may contain e.g. conservative substitutions or be truncated. The rationale for using the polypeptides of the invention is that the energy content due to the relatively small length of the peptide is low as compared to the effect on satiety.

Further protection of the termini, particularly the C-terminus of the polypeptides according to the invention, such as DC7-2, and the trypsin-sensitive lysine can be accomplished by either of
Modification;
extension;
specific or non-specific encapsulation; or
co-administration of endo- and exopeptidase inhibitors, either synthetic or naturally derived inhibitors.

Lysine (ASDKPYIL), a positive charge, e.g. —NH3+ at the N-terminus, and leucine (ASDKPYIL) or equivalent at the C-terminus are all required for high-affinity binding. Extensions of the C-terminus with the naturally occurring amino acid sequence of alpha-actinin-2: ASDKPYILA or ASDKPYILAE or ASDKPYILAEELRRELPP are all inactive, but are activated by pepsin or carboxypeptidases.

One approach in order to provide more protected peptides could be to combine longer version(s) of the DC7-2 sequence using papain digestion of the isolated alpha-actinin-2 (isopellet) with co-administration of naturally occurring protease inhibitors, such as from potato juice. Although many plant-derived protease inhibitors and actions of same are known, the potato protein isolate/extract (such as from side-stream production of potato starch) is particularly interesting because of a very high content of trypsin/chymotrypsin inhibitors (TI/CI, >20 kDa—up to ½ of total protein) as well as high content (up to 5%) of carboxypeptidase inhibitors (CPI, <5 kDa), both of which would appear useful for protection of DC7-2. Because of difference in size and thermal stability, heat-treated potato protein extract (PE, ‘Protamylasse’) contains primarily CPI, whereas potato protein isolate (PPI) obtained by filtration (>10 kDa) of potato juice from starch processing prior to heat-treatment contains active TI/CI. A combination of the two fractions may provide for maximal protection.

A potato protein extract suitable for the present invention may be provided as described in e.g. US2007/0148267, or as described in Nakajima S1 et al. J Agric Food Chem. 2011 Sep. 14; 59(17): 9491-6.

### Definitions

When terms such as “one”, “a” or “an” are used in this disclosure they mean “at least one”, or “one or more” unless otherwise indicated. Further, the term “comprising” is intended to mean “including” and thus allows for the presence of other constituents, features, conditions, or steps than those explicitly recited.

In some specific embodiments, the first 1, 2, or 3 amino acids in the N-terminal of the amino acid sequences according to the invention are in the D-form. It is assumed that the N-terminal trimming and thereby degradation of the peptides are somewhat delayed by having amino acids of the D-form in the N-terminal of these polypeptides. Alternatively and in some embodiments, the first 1, 2, or 3 amino acids in the N-terminal of the amino acid sequences according to the invention are amino acids in beta or gamma forms. Beta amino acids have their amino group bonded to the beta carbon rather than the alpha carbon as in the 20 standard natural amino acids. A capital D-letter subscript after the letter representing the amino acid residue designate herein amino acids specified to be in D-form, such as W_{D} referring to a tryptophan in D-form. A capital L-letter subscript after the letter representing the amino acid residue designate herein amino acids specified to be in L-form, such as WL referring to a tryptophan in L-form. If not otherwise indicated, an amino acid is in its natural L-form.

Alternatively, the first 1, 2, or 3 amino acids in the N-terminal of the amino acid sequences according to the invention may be modified by incorporation of protective groups, e.g. fluorine, or alternatively cyclic amino acids or other suitable non-natural amino acids are used.

A “variant” or “analogue” of a peptide refers to a peptide having an amino acid sequence that is substantially identical to a reference peptide, typically a native or “parent” polypeptide, or a polypeptide of formula I or II. The peptide variant may possess one or more amino acid substitutions, deletions, and/or insertions at certain positions within the native amino acid sequence. The “variant” within this definition still has functional activity. In some embodiment a variant has at least 80% sequence identity with the reference polypeptide. In some embodiments a variant has at least 85% sequence identity with the reference polypeptide. In other embodiments a variant has at least 90% sequence identity with the reference polypeptide. In a further embodiment a variant has at least 95% sequence identity with the reference polypeptide.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). A particular form of conservative amino acid substitutions include those with amino acids, which are not among the normal 20 amino acids encoded by the genetic code. Since preferred embodiments of the present invention entail use of synthetic peptides, it is unproblematic to provide such “non-naturally occurring” amino acid residues in the peptides disclosed herein, and thereby it is possible to exchange the natural saturated carbon chains in the side chains of amino acid residues with shorter or longer saturated carbon chains—for instance, lysine may be substituted with an amino acid having a side chain—(CH2)nNH3, where n is different from 4, and arginine may be substituted with an amino acid having the side chain (CH2)nNHC(═NH2)NH2, where n is different from 3, etc. Similarly, the acidic amino acids aspartic acid and glutamic acid may be substituted with amino acid residues having the side chains—(CH₂)nCOOH, where n>2.

The polypeptides of this invention may in some embodiments benefit from having higher stability than polypeptides containing only naturally occurring amino acids, and its modification enables to have much higher stability, such as a modification in the N-terminal of the polypeptide.

Accordingly and in some embodiments, the polypeptides of this invention have at their N-terminal a protection group, such as a protection group selected from the group consisting of acetyl group, fluorenyl methoxy carbonyl group, formyl group, palmitoyl group, myristyl group, stearyl group and polyethylene glycol (PEG).

The active peptide may also be di- or multimerized, e.g. through cross-linking with suitable di- or multivalent chemical cross-linkers, e.g. disuccinimidyl suberate, containing spacers of different length, e.g. 10-100 Å, and different functionality, e.g. homo- or heterofunctional, for coupling through non-critical amino or other reactive groups. Alternatively, photoactivation or enzymatic cross-linking may be used to increase stability and potency in vivo.

The modifications of peptides described above greatly increase the stability of the peptides of this invention. The term used herein “stability” refers to in vivo stability, such as the stability in the gut of a subject receiving such polypeptide. The protection group described above protects the peptides from the attack of protease in vivo.

The polypeptides according to the invention may be derived from a proteolytic digests of meat and be resistant to pepsin degradation. Accordingly, in some embodiments a polypeptide according to the invention may only contain naturally occurring amino acids.

In other embodiments, a polypeptide according to the invention is more stable towards degradation in the gastrointestinal tract, e.g. as measured in a stability assay described in the examples of the present invention, as compared to a control peptide. In some embodiments, a polypeptide according to the invention is more stable towards degradation in the gastrointestinal tract, e.g. measured in a stability assay described in the examples of the present invention as compared to a control peptide with the sequence RRPYIL.

In some embodiments, a polypeptide according to the invention has an half-life (T½) of degradation in vivo in the gut or in vitro, e.g. measured in a stability assay described in the examples of the present invention, which is higher than 2 min, such as higher than 4 min, such as higher than 6 min, such as higher than 8 min, such as higher than 10 min, such as higher than 15 min, such as higher than 20 min, such as higher than 25 min, such as higher than 30 min, such as higher than 35 min, such as higher than 40 min, such as higher than 45 min, such as higher than 50 min, such as higher than 55 min, such as higher than 60 min.

The term “substantially identical” in the context of two amino acid sequences means that the sequences, when optimally aligned, such as by the programs GAP or BESTFIT using default gap weights, share at least about 50, at least about 60, at least about 70, at least about 80, at least about 90, at least about 95, at least about 98, or at least about 99 percent sequence identity. In some embodiments, when measuring the sequence identity between two different peptide sequences, a gap of one or two amino acids is allowed when the two peptide sequences are aligned without having any influence on the value of sequence identity. In some embodiments, a residue position that is not identical differ by only a conservative amino acid substitution. Sequence identity is typically measured using sequence analysis software. Protein analysis software matches similar sequences using measures of similarity assigned to various substitutions, deletions and other modifications, including conservative amino acid substitutions. For instance, the publicly available GCG software contains programs such as “Gap” and “BestFit” which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences can also be compared using FASTA or ClustalW, applying default or recommended parameters. A program in GCG Version 6.1., FASTA (e.g., FASTA2 and FASTA3) provides alignments and percent sequence identity of the regions of the best overlap between the query and search sequences (Pearson, Methods Enzymol. 1990; 183:63-98; Pearson, Methods Mol. Biol. 2000; 132:185-219). Another preferred algorithm when comparing a sequence to a database containing a large number of sequences from various organisms is the computer program BLAST, especially blastp, using default parameters. See, e.g., Altschul et al., J. Mol. Biol. 1990; 215:403-410; Altschul et al., Nucleic Acids Res. 1997; 25:3389-402 (1997); each herein incorporated by reference. “Corresponding” amino acid positions in two substantially identical amino acid sequences are those aligned by any of the protein analysis software mentioned herein, typically using default parameters.

The term “functional activity” as used herein refers to a polypeptide that stimulates cell signalling measured as fluorescence by elevated intracellular calcium or cellular release of gut hormones, such as measured in the signalling assays described in the examples. The functional activity of a variant may exhibit at least about 25%, such as at least about 50%, such as at least about 75%, such as at least about 90% of the specific activity of a reference polypeptide, such as the octapeptide ASDKPYIL, when tested in the assays as described herein. Alternatively, the functional activity of a variant may exhibit higher activity than a reference polypeptide, such as the octapeptide ASDKPYIL, when tested in the assays as described herein.

An “isolated” molecule is a molecule that is the predominant species in the composition wherein it is found with respect to the class of molecules to which it belongs (i.e., it makes up at least about 5% of the type of molecule in the composition and typically will make up at least about 10%, at least about 20%, at least about 30%, at least about 40%, at least about 50%, at least about 60%, at least about 70%, at least about 80%, at least about 85%, at least about 90%, at least about 95%, or more of the species of molecules, e.g., peptides, in the composition). Commonly, a composition of a specific peptide sequence may exhibit 90%-99% homogeneity for peptides in the context of all present peptide species in the composition or at least with respect to substantially active peptide species in the context of proposed use. If produced synthetically, a composition of a specific peptide sequence will exhibit 98%-99%, or even higher and close to 100% homogeneity for peptides in the context of all present peptide species in the composition or at least with respect to substantially active peptide species in the context of proposed use.

Unless otherwise indicated the polypeptides within the present invention is a linear sequence of amino acids. The term “linear sequence” as used herein refers to the specific sequence of amino acids connected by standard peptide bonds in standard N- to C-terminal direction. The peptide may contain only peptide bonds. In some embodiments however, a second part of a peptide sequence may be bound to and continue from the side chain of a terminal amino acid in a first part of an amino acid sequence. Also the term does not exclude that an amino acid within a sequence, such as within AA1-AA8, may be connected, such as through the side chains, with another amino acid at a distant location within the peptide sequence, such as a distant location within AA1-AA8.

In the context of the present invention, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

The terms “patient” and “subject” refer to any human or animal that may be treated using the methods of the present invention.

Many aspect of the present invention relates to the use of polypeptides or compositions to promote satiety in a subject. The underlying cause of a metabolic syndrome or disorder that may treated by the polypeptides or compositions according to the invention, is an overconsumption of calories, while still not feeling satiety. By inducing or promoting satiety or reducing feed intake in a subject, such total amounts of calories, including calories derived from fat and carbohydrates are reduced in the subject. Accordingly, the polypeptides and compositions of the invention may be used in preventing or reducing a metabolic syndrome or disorder, such as obesity, insulin-deficiency or insulin-resistance related disorders, Diabetes Mellitus (such as, for example, Type 2 Diabetes), glucose intolerance, abnormal lipid metabolism, atherosclerosis, hypertension, cardiac pathology, stroke, non-alcoholic fatty liver disease, hyperglycemia, hepatic steatosis, dyslipidemia, dysfunction of the immune system associated with overweight and obesity, cardiovascular diseases, high cholesterol, elevated triglycerides, asthma, sleep apnoea, osteoarthritis, neuro-degeneration, gallbladder disease, syndrome X, inflammatory and immune disorders, atherogenic dyslipidemia and cancer.

As used herein a “protease inhibitor” may be any inhibitor of protease activity from proteases, such as trypsin, chymotrypsin, and carboxypeptidases, such as Carboxypeptidase A and B. A suitable protease inhibitor may be derived from plant protease inhibitors, such as from soybeans or potato. One specific suitable protease inhibitor is potato carboxypeptidase inhibitor (PCI), a 39-residue protein which inhibits all mammalian members of the A/B subfamily of carboxypeptidases in a substrate-like manner through interaction of the C-terminus of the PCI protein. Other suitable protease inhibitors, that may be derived from potato tubers include chymotrypsin inhibitors and Potato Kunitz inhibitor-1, a potent inhibitor of the animal pancreatic proteinase trypsin. A “protease inhibitor” may be KMC Potato Protein and KMC Potato Extract as such.

As used herein “potato proteinase inhibitor II (PI2)” refers to the Type II proteinase inhibitors (PI2) derived from potato tubers. This included PI2 extracted from potato tubers, such as a potato protein extract, as well as PI2 produced recombinantly in e.g. E. coli or any other host for recombinant expression.

As used herein the term “potato protein extract” refers to an extract of the potato (Solanum tuberosum) with a concentration of potato proteinase inhibitor (PPI) II higher than 1, 2, 3, 4, 5, 6, 7, 8, 9, or 10%. A potato protein extract prepared as disclosed herein from acidified potato tuber juice may typically yield about 0.15-0.25 mg total protein per gram of potato tuber fresh weight. A potato protein extract may be obtained by a method as described in any one of Komarnytsky et al. (Int J Obes (Lond). 2011 February; 35(2): 236-243), HPF Peters et al. (International Journal of Obesity (2011) 35, 244-250), or SAE KWANG K U et al. (EXPERIMENTAL AND THERAPEUTIC MEDICINE 12: 354-364, 2016).

As used herein the term “a gastrointestinal peptide hormone” refers to any peptide hormone secreted by enteroendocrine cells in the stomach, pancreas, or small intestine to exert an autocrine or paracrine actions controlling functions of the digestive organs or actions as neurotransmitters and neuromodulators in the central and peripheral nervous systems. Included within this definition is Cholecystokinin (CCK), Gastrin, Secretin, Vasoactive Intestinal Peptide (VIP), Glucose-dependent insulinotropic peptide (GIP), Glucagon-like Peptide 1 and 2 (GLP-1 and -2), Bombesin, Chromogranin A, Glucagon, Insulin, Leptin, Neuropeptide Y, Neurotensin, Neuromedin, Pancreatic Polypeptide, PYY, Amylin, Oxyntomodulin, Xexin, Motilin, Grehlin, and Somatostatin, as well as any bioactive analogue or variant of any one of these peptide hormones.

As used herein the term “protein substrate” refers to any protein or composition comprising one or more proteins that may serve as a substrate for proteases, such as trypsin, chymotrypsin, and carboxypeptidases, such as Carboxypeptidase A and B. Included within this definition is any general protein source from animal, such as meat, such as bovine heart, such as from blood or plasma, or from a milk product, such as whey, from a plant, such as cereals, such as wheat, legumes, soy, rice, nuts, seeds, from micro or macroalgae, such as Spirulina, or from a microorganism, such as bacteria, such as Bacillus sp. Or from fungi, such as yeast, such as Saccharomyces sp. The term is not intended to include a specific polypeptide or gastrointestinal peptide hormone according to the invention described herein.

In some embodiments, the protein substrate as used herein is different from a protein substrate derived from potato, such as a potato extract.

As used herein the term “crude protein hydrolysate” refers to any hydrolysate of a composition or product obtained from an animal or plant source with a content of protein. Included within this term are hydrolysates derived from meat, such as bovine heart, from blood or plasma, or from plants, such as whey or rice.

### Preparation of Polypeptides of the Invention

The invention also relates to a method of preparing polypeptides of the invention as mentioned above. The method of synthesis or preparation thereof includes, but is not limited to recombinant (whether produced from cDNA, genomic DNA, synthetic DNA or other form of nucleic acid), synthetic, and transgenic means.

The polypeptides of the invention described herein may be produced by means of recombinant nucleic acid techniques. In general, a nucleic acid sequence encoding the desired polypeptide is then inserted into an expression vector, which is in turn transformed or transfected into host cells.

As an alternative and also the preferred option, the polypeptides of the invention are produced by synthetic means, i.e. by polypeptide synthesis. In some embodiments, the invention relates to a method of manufacturing an analogue comprising non-natural amino acids from about 5 total residues to about 20 total residues. In some embodiments, an analogue comprises 1, 2, 3, 4, 5, 6, 7, 8, 9, 10, 11, 12, 13, 14, 15, 16, 17, 18, 19, or 20 non-natural amino acids, such as any one of the following non-naturally occurring amino acid residues:

The polypeptides of the present invention can also comprise non-naturally occurring amino acid residues. Non-naturally occurring amino acids include, without limitation, beta-alanine, desaminohistidine, trans-3-methylproline, 2,4-methanoproline, cis-4-hydroxyproline, trans-4-hydroxyproline, N-methylglycine, allo-threonine, methylthreonine, hydroxyethylcys-teine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, nor-valine, 2-azaphenylalanine, 3-azaphenylalanine, 4-azaphenylalanine, and 4-fluorophenylalanine. Several methods are known in the art for incorporating non-naturally occurring amino acid residues into polypeptides. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Polypeptides are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cul-tured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

As another alternative to synthetic preparation, the polypeptides of the invention may be purified from any natural source containing such polypeptide, such as from the proteolytic hydrolysate of muscle tissue, such as by the methods described in the example section.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). They may be purified by affinity chromatography on an antibody column. Additional purification may be achieved by conventional chemical purification means, such as high performance liquid chromatography. Other methods of purification, including barium citrate precipitation, are known in the art, and may be applied to the purification—see, for example, Scopes, R., Protein Purification, Springer-Verlag, N.Y., 1982.

For the methods of the invention including the therapeutic purposes it is not critical to have a high purity of a specific peptide of the invention. However, the higher the concentration of a specific peptide of the invention the higher is the effect in terms of inducing satiation and satiety relative to amount of total protein and total amount of calories consumed by the subject receiving the composition of polypeptides. It is to be understood that the idea of the invention is to administer polypeptides that induce satiation or satiety without administering a lot of calories to the subject.

In some embodiments the compositions of polypeptides of the invention are substantially pure. Thus, in an embodiment of the invention the polypeptides of the invention are purified to at least about 90 to 95% homogeneity, preferably to at least about 98% homogeneity. Purity may be assessed by e.g. HPLC and amino-terminal amino acid sequencing.

Administration and Pharmaceutical Compositions

Administration of the polypeptides according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Some kind of oral administration is preferred since these types of polypeptides are derived from a source that naturally has to pass through the mouth and to the intestinal mucosa.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

One of skill in the art will recognize that the appropriate dosage of the compositions and pharmaceutical compositions may vary depending on the individual being treated and the purpose. For example, the age, body weight, and medical history of the individual patient may affect the therapeutic efficacy of the therapy. Further, a lower dosage of the composition may be needed to produce a transient cessation of symptoms, while a larger dose may be needed to produce a complete cessation of symptoms associated with the disease, disorder, or indication. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response. Dosages may also depend on the strength of the particular polypeptide of the invention chosen for the pharmaceutical composition.

The dose of the composition or pharmaceutical compositions may vary. The dose of the composition may be once per day. In some embodiments, multiple doses may be administered to the subject per day. In some embodiments, the total dosage is administered in at least two application periods, In some embodiments, the period can be an hour, a day, a month, a year, a week, or a two-week period. In an additional embodiment of the invention, the total dosage is administered in two or more separate application periods, or separate doses.

In some embodiments, subjects can be administered the composition in which the composition is provided in a daily dose range of about 0.0001 mg/kg to about 5000 mg/kg of the weight of the subject. The dose administered to the subject can also be measured in terms of total amount of polypeptide of the invention administered per day. In some embodiments, a subject is administered from about 0.001 to about 3000 milligrams of polypeptide of the invention per day. In some embodiments a subject is administered up to about 2000 milligrams of polypeptide of the invention per day. In some embodiments, a subject is administered up to about 1800 milligrams of polypeptide of the invention per day. In some embodiments, a subject is administered up to about 1600 milligrams of polypeptide of the invention per day. In some embodiments, a subject is administered up to about 1400 milligrams of polypeptide of the invention per day. In some embodiments, a subject is administered up to about 1200 milligrams of polypeptide of the invention per day. In some embodiments, a subject is administered up to about 1000 milligrams of polypeptide of the invention per day. In some embodiments, a subject is administered up to about 800 milligrams of polypeptide of the invention per day. In some embodiments, a subject is administered from about 0.001 milligrams to about 700 milligrams of polypeptide of the invention per dose. In some embodiments, a subject is administered up to about 700 milligrams of polypeptide of the invention per dose. In some embodiments, a subject is administered up to about 600 milligrams of polypeptide of the invention per dose. In some embodiments, a subject is administered up to about 500 milligrams of polypeptide of the invention per dose. In some embodiments, a subject is administered up to about 400 milligrams of polypeptide of the invention per dose. In some embodiments, a subject is administered up to about 300 milligrams of secretin polypeptide of the invention per dose. In some embodiments, a subject is administered up to about 200 milligrams of polypeptide of the invention per dose. In some embodiments, a subject is administered up to about 100 milligrams of polypeptide of the invention per dose. In some embodiments, a subject is administered up to about 50 milligrams of polypeptide of the invention per dose.

A composition, wherein a polypeptide of the invention is added may be any food composition, food product, or food ingredient. Here, the term “food” is used in a broad sense—and covers food for humans as well as food for animals (i.e. a feed). In a preferred aspect, the food is for human consumption. The food may be in the form of a solution or as a solid—depending on the use and/or the mode of application and/or the mode of administration.

When used as—or in the preparation of—a food—such as functional food—the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

The composition of the present invention may be used as a food ingredient.

As used herein the term “food ingredient” includes a formulation which is or can be added to functional foods or foodstuffs as a nutritional supplement. The term food ingredient as used here also refers to formulations which can be used at low levels in a wide variety of products that require gelling, texturising, stabilising, suspending, film-forming and structuring, retention of juiciness and improved mouthfeel, without adding viscosity.

The food ingredient may be in the form of a solution or as a solid—depending on the use and/or the mode of application and/or the mode of administration.

The composition of the present invention may be—or may be added to—food supplements.

The composition of the present invention may be—or may be added to—functional foods.

As used herein, the term “functional food” means food which is capable of providing not only a nutritional effect and/or a taste satisfaction, but is also capable of delivering a further beneficial effect to consumer.

Accordingly, functional foods are ordinary foods that have components or ingredients (such as those described herein) incorporated into them that impart to the food a specific functional—e.g. medical or physiological benefit—other than a purely nutritional effect.

Although there is no legal definition of a functional food, most of the parties with an interest in this area agree that they are foods marketed as having specific health effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

Surveys have suggested that consumers place the most emphasis on functional food claims relating to heart disease. Preventing cancer is another aspect of nutrition which interests consumers a great deal, but interestingly this is the area that consumers feel they can exert least control over. In fact, according to the World Health Organization, at least 35% of cancer cases are diet-related. Furthermore claims relating to osteoporosis, gut health and obesity effects are also key factors that are likely to incite functional food purchase and drive market development.

The composition of the present invention can be used in the preparation of or added to food products such as one or more of: jams, marmalades, jellies, dairy products (such as milk or cheese), meat products, poultry products, fish products, vegetable-based soups, and bakery products.

By way of example, the composition of the present invention can be used as ingredients to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, breakfast cereals, instant noodles and cup noodles, instant soups and cup soups, balanced foods and drinks, sweeteners, texture improved snack bars, fibre bars, bake stable fruit fillings, care glaze, chocolate bakery filling, cheese cake flavoured filling, fruit flavoured cake filling, cake and doughnut icing, heat stable bakery filling, instant bakery filling creams, filing for cookies, ready-to-use bakery filling, reduced calorie filling, adult nutritional beverage, acidified soy/juice beverage, aseptic/retorted chocolate drink, bar mixes, beverage powders, calcium fortified soy/plaim and chocolate milk, calcium fortified coffee beverage.

A composition according to the present invention can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped tipping, low fat & lite natural cheddar cheese, low fat Swiss style yoghurt, aerated frozen desserts, and novelty bars, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

For certain aspects, preferably the foodstuff is a beverage.

For certain aspects, preferably the foodstuff is a bakery product—such as bread, Danish pastry, biscuits or cookies.

The present invention also provides a method of preparing a food or a food ingredient, the method comprising mixing a polypeptide according to the present invention or the composition according to the present invention with another food ingredient.

### Specific Embodiments of the Invention

One aspect of the invention related to an isolated polypeptide comprising the amino acid sequence

(formula I, SEQ ID NO: 1)
AA1-AA2-AA3-KAA4-AA5-AA6-AA7-AA8-E*,

wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; wherein E* is C-terminal extension with 1-10 of any amino acids; which polypeptide is not more than 50 amino acids in length; or a variant thereof with a sequence identity of at least 80%;

or an isolated polypeptide consisting of the amino acid sequence

(formula II, SEQ ID NO: 2)
R1-AA1-AA2-AA3-KAA4-AA5-AA6-AA7-AA8-E*-R2,

wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; R1 defines the N-term (—NH2) or a protection group; wherein E* is C-terminal extension with 1-10 of any amino acids; R2 defines the C-term (—COOH).

In some embodiments E* is 1-10 amino acids of the naturally occurring amino acid sequence of alpha-actinin-2.

In some embodiments E* is selected from A, AE, AEE, AEEL (SEQ ID NO: 1016), AEELR (SEQ ID NO:1017), AEELRR (SEQ ID NO:1018), AEELRRE (SEQ ID NO:1019), AEELRREL (SEQ ID NO:1020), AEELRRELP (SEQ ID NO:1021), and AEELRRELPP (SEQ ID NO:1022).

In some embodiments said polypeptide comprises or consist of AA1-AA2-AA3-K-P-Y-I-L-E*. In some embodiments said polypeptide comprises or consist of AA1-AA2-AA3-AA4-P-Y-I-L-E*.

In some embodiments AA1 is absent. In some embodiments AA1 is any one natural amino acid selected from Y, W, V, T, S, R, Q, P, N, M, L, K, I, H, G, F, E, D, C, and A. In some embodiments AA2 is absent. In some embodiments AA2 is any one natural amino acid selected from Y, W, V, T, S, R, Q, P, N, M, L, K, I, H, G, F, E, D, C, and A. In some embodiments AA2 when present is an amino acid selected from S, T, A, N, E and D. In some embodiments AA2 when present is an amino acid selected from S, T, G, A, N, E and D. In some embodiments AA3 is absent. In some embodiments AA1 is present. In some embodiments AA2 is present. In some embodiments AA3 is present. In some embodiments AA1 is A. In some embodiments AA2 is S. In some embodiments AA3 is D. In some embodiments AA3 is selected from any one amino acid C, D, E, N, P, and Q. In some embodiments AA3 is selected from D, E and G. In some embodiments AA3 is selected from E and G. In some embodiments AA3 is P. In some embodiments AA3 is C. In some embodiments AA4 is K. In some embodiments AA5 is P. In some embodiments AA5 is selected from P, S, D, A, T, K, and G. In some embodiments AA6 is selected from Y, N, I, and W. In some embodiments AA8 is selected from L, I, V, S, and M. In some embodiments AA6 is Y. In some embodiments AA7 is I. In some embodiments AA8 is L. In some embodiments AA6 is selected from Y and W. In some embodiments AA7 is selected from I and L. In some embodiments the amino acid sequence is not found in nature.

In some embodiments the amino acid sequence only contains natural amino acids.

In some embodiments the peptide is 5-50, such as 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, such as 5-18, such as 5-17, such as 5-16, such as 5-15, such as 5-14, such as 5-13, such as 5-12, such as 5-11, such as 5-10, such as 5-9, such as 5-8, such as 5-7, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments the sequence is 5-50, such as 6-50, such as 7-50, such as 8-50, such as 9-50, such as 10-50, such as 11-50, such as 12-50, such as 13-50, such as 14-50, such as 15-50, such as 16-50, such as 17-50, such as 18-50, such as 19-50, such as 20-50, such as 21-50, such as 22-50, such as 23-50, such as 24-50, such as 25-50, such as 26-50, such as 27-50, such as 28-50, such as 29-50, such as 30-50, such as 31-50, such as 32-50, such as 33-50, such as 34-50, such as 35-50, such as 36-50, such as 37-50, such as 38-50, such as 39-50, such as 40-50, such as 41-50, such as 42-50, such as 43-50, such as 44-50, such as 45-50, such as 46-50, such as 47-50, such as 48-50, such as 49-50 amino acids in length.

In some embodiments the polypeptide used in compositions according to the invention has or comprises a sequence selected from ASDKPYIL (SEQ ID NO:6), SDKPYIL (SEQ ID NO:7), DKPYIL (SEQ ID NO:8), and KPYIL (SEQ ID NO:9).

In some embodiments the polypeptide used in compositions according to the invention has or comprises a sequence selected from ASDKPYIL (SEQ ID NO:6), SDKPYIL (SEQ ID NO:7), DKPYIL (SEQ ID NO:8), KPYIL (SEQ ID NO:9), AGDKNYIL (SEQ ID NO:10), AGDKNYIT (SEQ ID NO:11), AGDKSYIT (SEQ ID NO:12), ADGKPYIV (SEQ ID NO:13), AEDKDFIT (SEQ ID NO: 14), AADKPYIL (SEQ ID NO:15), ATDKPYIL (SEQ ID NO: 16), AGDKPYIT (SEQ ID NO:17), ASEKPYIL (SEQ ID NO:18), ADGKPYVT (SEQ ID NO:19), AGDKPYIL (SEQ ID NO:20), ASDKPNIL (SEQ ID NO:21), ASDKPYIT (SEQ ID NO:22), AADKPFIL (SEQ ID NO:23), ASDKAYIT (SEQ ID NO:24), AGDKAYIT (SEQ ID NO:25), ANGKPFIT (SEQ ID NO:26), AGDKNFIT (SEQ ID NO:27), ASDKSYIT (SEQ ID NO:28), ASDKTYIT (SEQ ID NO:29), ASDKNYIT (SEQ ID NO:30), AGDKKYIT (SEQ ID NO:31), AGDKNYIS (SEQ ID NO:32), AADKNYIT (SEQ ID NO:33), AGDKNYIM (SEQ ID NO:34), AADKNFIM (SEQ ID NO:35), AADKNFIT (SEQ ID NO:36), and AGDKGIRS (SEQ ID NO:37).

In some embodiments the polypeptide according to the invention or used in compositions according to the invention has or comprises a sequence selected from ASDKPYILA (SEQ ID NO: 1006), ASDKPYILAE (SEQ ID NO:1007), ASDKPYILAEE (SEQ ID NO:1008), ASDKPYILAEEL (SEQ ID NO:1009), ASDKPYILAEELR (SEQ ID NO:1010), ASDKPYILAEELRR (SEQ ID NO:1011), ASDKPYILAEELRRE (SEQ ID NO:1012), ASDKPYILAEELRREL (SEQ ID NO:1013), ASDKPYILAEELRRELP (SEQ ID NO:1014), and ASDKPYILAEELRRELPP (SEQ ID NO:1015).

In some embodiments the polypeptide is an isolated polypeptide.

In some embodiments the polypeptide is synthetically made.

In some embodiments the polypeptide is a purified fragment.

In some embodiments the polypeptide is purified from animal sources.

In some embodiments the polypeptide is generated by enzymatic treatment of proteins from animal sources.

In some embodiments the polypeptide has been modified by N terminal acylation or other protection groups.

One aspect of the invention related to the use of an isolated polypeptide comprising the amino acid sequence

(formula III, SEQ ID NO: 3)
AA1-AA2-AA3-AA4-AA5-AA6-AA7-AA8,

wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%.

In some embodiments AA1 is absent. In some embodiments AA2 is absent. In some embodiments AA3 is absent. In some embodiments AA1 is present. In some embodiments AA2 is present. In some embodiments AA3 is present. In some embodiments AA1 is A. In some embodiments AA2 is S. In some embodiments AA3 is D. In some embodiments AA4 is K. In some embodiments AA5 is P. In some embodiments AA6 is Y. In some embodiments AA7 is I. In some embodiments AA8 is L. In some embodiments the amino acid sequence is not found in nature.

In some embodiments the polypeptide for use in compositions according to the present invention does not comprise or consists of any one of the sequences AVTEKKYILYDFSVTS (SEQ ID NO:5), PRRPYIL (SEQ ID NO:38), RRPYIL (SEQ ID NO:39), RPYIL (SEQ ID NO:40), RRPWIL (SEQ ID NO:41), KRPYIL (SEQ ID NO:42), KKPYIL (SEQ ID NO:43), Adamantoyl-KPYIL (SEQ ID NO:9), H-Lys-psi(CH₂NH)Lys-Pro-Tyr-Ile-Leu-OH (SEQ ID NO:44). In some embodiments the polypeptide does not comprise derivatives of Lys. In some embodiments the polypeptide is not a derivative of KPYIL.

In some embodiments the amino acid sequence only contains natural amino acids.

In some embodiments the polypeptide for use in compositions according to the present invention is 5-50, such as 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, such as 5-18, such as 5-17, such as 5-16, such as 5-15, such as 5-14, such as 5-13, such as 5-12, such as 5-11, such as 5-10, such as 5-9, such as 5-8, such as 5-7, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments the polypeptide for use in compositions according to the present invention is 5-50, such as 6-50, such as 7-50, such as 8-50, such as 9-50, such as 10-50, such as 11-50, such as 12-50, such as 13-50, such as 14-50, such as 15-50, such as 16-50, such as 17-50, such as 18-50, such as 19-50, such as 20-50, such as 21-50, such as 22-50, such as 23-50, such as 24-50, such as 25-50, such as 26-50, such as 27-50, such as 28-50, such as 29-50, such as 30-50, such as 31-50, such as 32-50, such as 33-50, such as 34-50, such as 35-50, such as 36-50, such as 37-50, such as 38-50, such as 39-50, such as 40-50, such as 41-50, such as 42-50, such as 43-50, such as 44-50, such as 45-50, such as 46-50, such as 47-50, such as 48-50, such as 49-50 amino acids in length.

In some embodiments the polypeptide for use in compositions according to the present invention has or comprises a sequence selected from ASDKPYIL (SEQ ID NO:6), SDKPYIL (SEQ ID NO:7), DKPYIL (SEQ ID NO:8), and KPYIL (SEQ ID NO:9).

In some embodiments the polypeptide for use in compositions according to the present invention has or comprises a sequence selected from ASDKPYIL (SEQ ID NO:6), SDKPYIL (SEQ ID NO:7), DKPYIL (SEQ ID NO:8), KPYIL (SEQ ID NO:9), AGDKNYIL (SEQ ID NO:10), AGDKNYIT (SEQ ID NO:11), AGDKSYIT (SEQ ID NO:12), ADGKPYIV (SEQ ID NO:13), AEDKDFIT (SEQ ID NO:14), AADKPYIL (SEQ ID NO:15), ATDKPYIL (SEQ ID NO:16), AGDKPYIT (SEQ ID NO: 17), ASEKPYIL (SEQ ID NO: 18), ADGKPYVT (SEQ ID NO:19), AGDKPYIL (SEQ ID NO:20), ASDKPNIL (SEQ ID NO:21), ASDKPYIT (SEQ ID NO:22), AADKPFIL (SEQ ID NO:23), ASDKAYIT (SEQ ID NO:24), AGDKAYIT (SEQ ID NO:25), ANGKPFIT (SEQ ID NO:26), AGDKNFIT (SEQ ID NO:27), ASDKSYIT (SEQ ID NO:28), ASDKTYIT (SEQ ID NO:29), ASDKNYIT (SEQ ID NO:30), AGDKKYIT (SEQ ID NO:31), AGDKNYIS (SEQ ID NO:32), AADKNYIT (SEQ ID NO:33), AGDKNYIM (SEQ ID NO:34), AADKNFIM (SEQ ID NO:35), AADKNFIT (SEQ ID NO:36), and AGDKGIRS (SEQ ID NO:37).

In some embodiments the polypeptide for use in compositions according to the present invention is an isolated polypeptide.

In some embodiments the polypeptide for use in compositions according to the present invention is synthetically made.

In some embodiments the polypeptide for use in compositions according to the present invention is a purified fragment.

In some embodiments the polypeptide for use in compositions according to the present invention is purified from animal sources.

In some embodiments the polypeptide of the invention is generated by enzymatic treatment of proteins from animal sources.

In some embodiments the polypeptide for use in compositions according to the present invention has been modified by N terminal acylation or other chemical modifications to introduce protection groups.

In some specific embodiments, the polypeptide for use in compositions according to the present invention consists of or comprises an amino acid sequence selected from the group consisting of KPYIL, KPYII (SEQ ID NO:45), KPYIV (SEQ ID NO:46), KPYLL (SEQ ID NO:47), KPYLI (SEQ ID NO:48), KPYLV (SEQ ID NO:49), KPYVL (SEQ ID NO:50), KPYVI (SEQ ID NO:51), KPYVV (SEQ ID NO:52), KPWIL (SEQ ID NO:53), KPWII (SEQ ID NO:54), KPWIV (SEQ ID NO:55), KPWLL (SEQ ID NO:56), KPWLI (SEQ ID NO:57), KPWLV (SEQ ID NO:58), KPWVL (SEQ ID NO:59), KPWVI (SEQ ID NO:60), KPWVV (SEQ ID NO:61), RPYIL (SEQ ID NO:40), RPYII (SEQ ID NO:62), RPYIV (SEQ ID NO:63), RPYLL (SEQ ID NO:64), RPYLI (SEQ ID NO:65), RPYLV (SEQ ID NO:66), RPYVL (SEQ ID NO:67), RPYVI (SEQ ID NO:68), RPYVV (SEQ ID NO:69), RPWIL (SEQ ID NO:70), RPWII (SEQ ID NO:71), RPWIV (SEQ ID NO:72), RPWLL (SEQ ID NO:73), RPWLI (SEQ ID NO:74), RPWLV (SEQ ID NO:75), RPWVL (SEQ ID NO:76), RPWVI (SEQ ID NO:77), and RPWVV (SEQ ID NO:78).

In some specific embodiments, the polypeptide for use in compositions according to the present invention consist of or comprises an amino acid sequence selected from the group consisting of DKPYIL (SEQ ID NO:8), DKPYII (SEQ ID NO:79), DKPYIV (SEQ ID NO:80), DKPYLL (SEQ ID NO:81), DKPYLI (SEQ ID NO:82), DKPYLV (SEQ ID NO:83), DKPYVL (SEQ ID NO:84), DKPYVI (SEQ ID NO:85), DKPYVV (SEQ ID NO:86), DKPWIL (SEQ ID NO:87), DKPWII (SEQ ID NO:88), DKPWIV (SEQ ID NO:89), DKPWLL (SEQ ID NO:90), DKPWLI (SEQ ID NO:91), DKPWLV (SEQ ID NO:92), DKPWVL (SEQ ID NO:93), DKPWVI (SEQ ID NO:94), DKPWVV (SEQ ID NO:95), DRPYIL (SEQ ID NO:96), DRPYII (SEQ ID NO:97), DRPYIV (SEQ ID NO:98), DRPYLL (SEQ ID NO:99), DRPYLI (SEQ ID NO:100), DRPYLV (SEQ ID NO:101), DRPYVL (SEQ ID NO:102), DRPYVI (SEQ ID NO:103), DRPYVV (SEQ ID NO:104), DRPWIL (SEQ ID NO:105), DRPWII (SEQ ID NO:106), DRPWIV (SEQ ID NO:107), DRPWLL (SEQ ID NO:108), DRPWLI (SEQ ID NO:109), DRPWLV (SEQ ID NO:110), DRPWVL (SEQ ID NO:111), DRPWVI (SEQ ID NO:112), DRPWVV (SEQ ID NO:113), EKPYIL (SEQ ID NO:114), EKPYII (SEQ ID NO:115), EKPYIV (SEQ ID NO:116), EKPYLL (SEQ ID NO:117), EKPYLI (SEQ ID NO:118), EKPYLV (SEQ ID NO:119), EKPYVL (SEQ ID NO:120), EKPYVI (SEQ ID NO:121), EKPYVV (SEQ ID NO:122), EKPWIL (SEQ ID NO:123), EKPWII (SEQ ID NO:124), EKPWIV (SEQ ID NO:125), EKPWLL (SEQ ID NO:126), EKPWLI (SEQ ID NO:127), EKPWLV (SEQ ID NO:128), EKPWVL (SEQ ID NO:129), EKPWVI (SEQ ID NO:130), EKPWVV (SEQ ID NO:131), ERPYIL (SEQ ID NO:132), ERPYII (SEQ ID NO:133), ERPYIV (SEQ ID NO:134), ERPYLL (SEQ ID NO:135), ERPYLI (SEQ ID NO:136), ERPYLV (SEQ ID NO:137), ERPYVL (SEQ ID NO:138), ERPYVI (SEQ ID NO:139), ERPYVV (SEQ ID NO:140), ERPWIL (SEQ ID NO:141), ERPWII (SEQ ID NO:142), ERPWIV (SEQ ID NO:143), ERPWLL (SEQ ID NO:144), ERPWLI (SEQ ID NO:145), ERPWLV (SEQ ID NO:146), ERPWVL (SEQ ID NO:147), ERPWVI (SEQ ID NO:148), ERPWVV (SEQ ID NO:149), RKPYIL (SEQ ID NO:150), RKPYII (SEQ ID NO:151), RKPYIV (SEQ ID NO:152), RKPYLL (SEQ ID NO:153), RKPYLI (SEQ ID NO:154), RKPYLV (SEQ ID NO:155), RKPYVL (SEQ ID NO:156), RKPYVI (SEQ ID NO:157), RKPYVV (SEQ ID NO:158), RKPWIL (SEQ ID NO:159), RKPWII (SEQ ID NO:160), RKPWIV (SEQ ID NO:161), RKPWLL (SEQ ID NO:162), RKPWLI (SEQ ID NO:163), RKPWLV (SEQ ID NO:164), RKPWVL (SEQ ID NO:165), RKPWVI (SEQ ID NO:166), RKPWVV (SEQ ID NO:167), RRPYIL (SEQ ID NO:39), RRPYII (SEQ ID NO:168), RRPYIV (SEQ ID NO:169), RRPYLL (SEQ ID NO:170), RRPYLI (SEQ ID NO:171), RRPYLV (SEQ ID NO:172), RRPYVL (SEQ ID NO:173), RRPYVI (SEQ ID NO:174), RRPYVV (SEQ ID NO:175), RRPWIL (SEQ ID NO:41), RRPWII (SEQ ID NO:176), RRPWIV (SEQ ID NO:177), RRPWLL (SEQ ID NO:178), RRPWLI (SEQ ID NO:179), RRPWLV (SEQ ID NO:180), RRPWVL (SEQ ID NO:181), RRPWVI (SEQ ID NO:182), RRPWVV (SEQ ID NO:183), GKPYIL (SEQ ID NO:184), GKPYII (SEQ ID NO:185), GKPYIV (SEQ ID NO:186), GKPYLL (SEQ ID NO:187), GKPYLI (SEQ ID NO:188), GKPYLV (SEQ ID NO:189), GKPYVL (SEQ ID NO:190), GKPYVI (SEQ ID NO:191), GKPYVV (SEQ ID NO:192), GKPWIL (SEQ ID NO:193), GKPWII (SEQ ID NO:194), GKPWIV (SEQ ID NO:195), GKPWLL (SEQ ID NO:196), GKPWLI (SEQ ID NO:197), GKPWLV (SEQ ID NO:198), GKPWVL (SEQ ID NO:199), GKPWVI (SEQ ID NO:200), GKPWVV (SEQ ID NO:201), GRPYIL (SEQ ID NO:202), GRPYII (SEQ ID NO:203), GRPYIV (SEQ ID NO:204), GRPYLL (SEQ ID NO:205), GRPYLI (SEQ ID NO:206), GRPYLV (SEQ ID NO:207), GRPYVL (SEQ ID NO:208), GRPYVI (SEQ ID NO:209), GRPYVV (SEQ ID NO:210), GRPWIL (SEQ ID NO:211), GRPWII (SEQ ID NO:212), GRPWIV (SEQ ID NO:213), GRPWLL (SEQ ID NO:214), GRPWLI (SEQ ID NO:215), GRPWLV (SEQ ID NO:216), GRPWVL (SEQ ID NO:217), GRPWVI (SEQ ID NO:218), and GRPWVV (SEQ ID NO:219).

In some specific embodiments, the polypeptide for use in compositions according to the present invention consists of or comprises an amino acid sequence selected from the group consisting of SDKPYIL (SEQ ID NO:220), SDKPYII (SEQ ID NO:221), SDKPYIV (SEQ ID NO:222), SDKPYLL (SEQ ID NO:223), SDKPYLI (SEQ ID NO:224), SDKPYLV (SEQ ID NO:225), SDKPYVL (SEQ ID NO:226), SDKPYVI (SEQ ID NO:227), SDKPYVV (SEQ ID NO:228), SDKPWIL (SEQ ID NO:229), SDKPWII (SEQ ID NO:230), SDKPWIV (SEQ ID NO:231), SDKPWLL (SEQ ID NO:232), SDKPWLI (SEQ ID NO:233), SDKPWLV (SEQ ID NO:234), SDKPWVL (SEQ ID NO:235), SDKPWVI (SEQ ID NO:236), SDKPWVV (SEQ ID NO:237), SDRPYIL (SEQ ID NO:238), SDRPYII (SEQ ID NO:239), SDRPYIV (SEQ ID NO:240), SDRPYLL (SEQ ID NO:241), SDRPYLI (SEQ ID NO:242), SDRPYLV (SEQ ID NO:243), SDRPYVL (SEQ ID NO:244), SDRPYVI (SEQ ID NO:245), SDRPYVV (SEQ ID NO:246), SDRPWIL (SEQ ID NO:247), SDRPWII (SEQ ID NO:248), SDRPWIV (SEQ ID NO:249), SDRPWLL (SEQ ID NO:250), SDRPWLI (SEQ ID NO:251), SDRPWLV (SEQ ID NO:252), SDRPWVL (SEQ ID NO:253), SDRPWVI (SEQ ID NO:254), SDRPWVV (SEQ ID NO:255), SEKPYIL (SEQ ID NO:256), SEKPYII (SEQ ID NO:257), SEKPYIV (SEQ ID NO:258), SEKPYLL (SEQ ID NO:259), SEKPYLI (SEQ ID NO:260), SEKPYLV (SEQ ID NO:261), SEKPYVL (SEQ ID NO:262), SEKPYVI (SEQ ID NO:263), SEKPYVV (SEQ ID NO:264), SEKPWIL (SEQ ID NO:265), SEKPWII (SEQ ID NO:266), SEKPWIV (SEQ ID NO:267), SEKPWLL (SEQ ID NO:268), SEKPWLI (SEQ ID NO:269), SEKPWLV (SEQ ID NO:270), SEKPWVL (SEQ ID NO:271), SEKPWVI (SEQ ID NO:272), SEKPWVV (SEQ ID NO:273), SERPYIL (SEQ ID NO:274), SERPYII (SEQ ID NO:275), SERPYIV (SEQ ID NO:276), SERPYLL (SEQ ID NO:277), SERPYLI (SEQ ID NO:278), SERPYLV (SEQ ID NO:279), SERPYVL (SEQ ID NO:280), SERPYVI (SEQ ID NO:281), SERPYVV (SEQ ID NO:282), SERPWIL (SEQ ID NO:283), SERPWII (SEQ ID NO:284), SERPWIV (SEQ ID NO:285), SERPWLL (SEQ ID NO:286), SERPWLI (SEQ ID NO:287), SERPWLV (SEQ ID NO:288), SERPWVL (SEQ ID NO:289), SERPWVI (SEQ ID NO:290), SERPWVV (SEQ ID NO:291), TDKPYIL (SEQ ID NO:292), TDKPYII (SEQ ID NO:293), TDKPYIV (SEQ ID NO:294), TDKPYLL (SEQ ID NO:295), TDKPYLI (SEQ ID NO:296), TDKPYLV (SEQ ID NO:297), TDKPYVL (SEQ ID NO:298), TDKPYVI (SEQ ID NO:299), TDKPYVV (SEQ ID NO:300), TDKPWIL (SEQ ID NO:301), TDKPWII (SEQ ID NO:302), TDKPWIV (SEQ ID NO:303), TDKPWLL (SEQ ID NO:304), TDKPWLI (SEQ ID NO:305), TDKPWLV (SEQ ID NO:306), TDKPWVL (SEQ ID NO:307), TDKPWVI (SEQ ID NO:308), TDKPWVV (SEQ ID NO:309), TDRPYIL (SEQ ID NO:310), TDRPYII (SEQ ID NO:311), TDRPYIV (SEQ ID NO:312), TDRPYLL (SEQ ID NO:313), TDRPYLI (SEQ ID NO:314), TDRPYLV (SEQ ID NO:315), TDRPYVL (SEQ ID NO:316), TDRPYVI (SEQ ID NO:317), TDRPYVV (SEQ ID NO:318), TDRPWIL (SEQ ID NO:319), TDRPWII (SEQ ID NO:320), TDRPWIV (SEQ ID NO:321), TDRPWLL (SEQ ID NO:322), TDRPWLI (SEQ ID NO:323), TDRPWLV (SEQ ID NO:324), TDRPWVL (SEQ ID NO:325), TDRPWVI (SEQ ID NO:326), TDRPWVV (SEQ ID NO:327), TEKPYIL (SEQ ID NO:328), TEKPYII (SEQ ID NO:329), TEKPYIV (SEQ ID NO:330), TEKPYLL (SEQ ID NO:331), TEKPYLI (SEQ ID NO:332), TEKPYLV (SEQ ID NO:333), TEKPYVL (SEQ ID NO:334), TEKPYVI (SEQ ID NO:335), TEKPYVV (SEQ ID NO:336), TEKPWIL (SEQ ID NO:337), TEKPWII (SEQ ID NO:338), TEKPWIV (SEQ ID NO:339), TEKPWLL (SEQ ID NO:340), TEKPWLI (SEQ ID NO:341), TEKPWLV (SEQ ID NO:342), TEKPWVL (SEQ ID NO:343), TEKPWVI (SEQ ID NO:344), TEKPWVV (SEQ ID NO:345), TERPYIL (SEQ ID NO:346), TERPYII (SEQ ID NO:347), TERPYIV (SEQ ID NO:348), TERPYLL (SEQ ID NO:349), TERPYLI (SEQ ID NO:350), TERPYLV (SEQ ID NO:351), TERPYVL (SEQ ID NO:352), TERPYVI (SEQ ID NO:353), TERPYVV (SEQ ID NO:354), TERPWIL (SEQ ID NO:355), TERPWII (SEQ ID NO:356), TERPWIV (SEQ ID NO:357), TERPWLL (SEQ ID NO:358), TERPWLI (SEQ ID NO:359), TERPWLV (SEQ ID NO:360), TERPWVL (SEQ ID NO:361), TERPWVI (SEQ ID NO:362), and TERPWVV (SEQ ID NO:363).

In some specific embodiments, the polypeptide for use in compositions according to the present invention consists of or comprises an amino acid sequence selected from the group consisting of ASDKPYII (SEQ ID NO:364), ASDKPYIV (SEQ ID NO:365), ASDKPYLL (SEQ ID NO:366), ASDKPYLI (SEQ ID NO:367), ASDKPYLV (SEQ ID NO:368), ASDKPYVL (SEQ ID NO:369), ASDKPYVI (SEQ ID NO:370), ASDKPYVV (SEQ ID NO:371), ASDKPWIL (SEQ ID NO:372), ASDKPWII (SEQ ID NO:373), ASDKPWIV (SEQ ID NO:374), ASDKPWLL (SEQ ID NO:375), ASDKPWLI (SEQ ID NO:376), ASDKPWLV (SEQ ID NO:377), ASDKPWVL (SEQ ID NO:378), ASDKPWVI (SEQ ID NO:379), ASDKPWVV (SEQ ID NO:380), ASDRPYIL (SEQ ID NO:381), ASDRPYII (SEQ ID NO:382), ASDRPYIV (SEQ ID NO:383), ASDRPYLL (SEQ ID NO:384), ASDRPYLI (SEQ ID NO:385), ASDRPYLV (SEQ ID NO:386), ASDRPYVL (SEQ ID NO:387), ASDRPYVI (SEQ ID NO:388), ASDRPYVV (SEQ ID NO:389), ASDRPWIL (SEQ ID NO:390), ASDRPWII (SEQ ID NO:391), ASDRPWIV (SEQ ID NO:392), ASDRPWLL (SEQ ID NO:393), ASDRPWLI (SEQ ID NO:394), ASDRPWLV (SEQ ID NO:395), ASDRPWVL (SEQ ID NO:396), ASDRPWVI (SEQ ID NO:397), ASDRPWVV (SEQ ID NO:398), ASEKPYIL (SEQ ID NO:399), ASEKPYII (SEQ ID NO:400), ASEKPYIV (SEQ ID NO:401), ASEKPYLL (SEQ ID NO:402), ASEKPYLI (SEQ ID NO:403), ASEKPYLV (SEQ ID NO:404), ASEKPYVL (SEQ ID NO:405), ASEKPYVI (SEQ ID NO:406), ASEKPYVV (SEQ ID NO:407), ASEKPWIL (SEQ ID NO:408), ASEKPWII (SEQ ID NO:409), ASEKPWIV (SEQ ID NO:410), ASEKPWLL (SEQ ID NO:411), ASEKPWLI (SEQ ID NO:412), ASEKPWLV (SEQ ID NO:413), ASEKPWVL (SEQ ID NO:414), ASEKPWVI (SEQ ID NO:415), ASEKPWVV (SEQ ID NO:416), ASERPYIL (SEQ ID NO:417), ASERPYII (SEQ ID NO:418), ASERPYIV (SEQ ID NO:419), ASERPYLL (SEQ ID NO:420), ASERPYLI (SEQ ID NO:421), ASERPYLV (SEQ ID NO:422), ASERPYVL (SEQ ID NO:423), ASERPYVI (SEQ ID NO:424), ASERPYVV (SEQ ID NO:425), ASERPWIL (SEQ ID NO:426), ASERPWII (SEQ ID NO:427), ASERPWIV (SEQ ID NO:428), ASERPWLL (SEQ ID NO:429), ASERPWLI (SEQ ID NO:430), ASERPWLV (SEQ ID NO:431), ASERPWVL (SEQ ID NO:432), ASERPWVI (SEQ ID NO:433), ASERPWVV (SEQ ID NO:434), ATDKPYIL (SEQ ID NO:435), ATDKPYII (SEQ ID NO:436), ATDKPYIV (SEQ ID NO:437), ATDKPYLL (SEQ ID NO:438), ATDKPYLI (SEQ ID NO:439), ATDKPYLV (SEQ ID NO:440), ATDKPYVL (SEQ ID NO:441), ATDKPYVI (SEQ ID NO:442), ATDKPYVV (SEQ ID NO:443), ATDKPWIL (SEQ ID NO:444), ATDKPWII (SEQ ID NO:445), ATDKPWIV (SEQ ID NO:446), ATDKPWLL (SEQ ID NO:447), ATDKPWLI (SEQ ID NO:448), ATDKPWLV (SEQ ID NO:449), ATDKPWVL (SEQ ID NO:450), ATDKPWVI (SEQ ID NO:451), ATDKPWVV (SEQ ID NO:452), ATDRPYIL (SEQ ID NO:453), ATDRPYII (SEQ ID NO:454), ATDRPYIV (SEQ ID NO:455), ATDRPYLL (SEQ ID NO:456), ATDRPYLI (SEQ ID NO:457), ATDRPYLV (SEQ ID NO:458), ATDRPYVL (SEQ ID NO:459), ATDRPYVI (SEQ ID NO:460), ATDRPYVV (SEQ ID NO:461), ATDRPWIL (SEQ ID NO:462), ATDRPWII (SEQ ID NO:463), ATDRPWIV (SEQ ID NO:464), ATDRPWLL (SEQ ID NO:465), ATDRPWLI (SEQ ID NO:466), ATDRPWLV (SEQ ID NO:467), ATDRPWVL (SEQ ID NO:468), ATDRPWVI (SEQ ID NO:469), ATDRPWVV (SEQ ID NO:470), ATEKPYIL (SEQ ID NO:471), ATEKPYII (SEQ ID NO:472), ATEKPYIV (SEQ ID NO:473), ATEKPYLL (SEQ ID NO:474), ATEKPYLI (SEQ ID NO:475), ATEKPYLV (SEQ ID NO:476), ATEKPYVL (SEQ ID NO:477), ATEKPYVI (SEQ ID NO:478), ATEKPYVV (SEQ ID NO:479), ATEKPWIL (SEQ ID NO:480), ATEKPWII (SEQ ID NO:481), ATEKPWIV (SEQ ID NO:482), ATEKPWLL (SEQ ID NO:483), ATEKPWLI (SEQ ID NO:484), ATEKPWLV (SEQ ID NO:485), ATEKPWVL (SEQ ID NO:486), ATEKPWVI (SEQ ID NO:487), ATEKPWVV (SEQ ID NO:488), ATERPYIL (SEQ ID NO:489), ATERPYII (SEQ ID NO:490), ATERPYIV (SEQ ID NO:491), ATERPYLL (SEQ ID NO:492), ATERPYLI (SEQ ID NO:493), ATERPYLV (SEQ ID NO:494), ATERPYVL (SEQ ID NO:495), ATERPYVI (SEQ ID NO:496), ATERPYVV (SEQ ID NO:497), ATERPWIL (SEQ ID NO:498), ATERPWII (SEQ ID NO:499), ATERPWIV (SEQ ID NO:500), ATERPWLL (SEQ ID NO:501), ATERPWLI (SEQ ID NO:502), ATERPWLV (SEQ ID NO:503), ATERPWVL (SEQ ID NO:504), ATERPWVI (SEQ ID NO:505), ATERPWVV (SEQ ID NO:506), LSDKPYIL (SEQ ID NO:507), LSDKPYII (SEQ ID NO:508), LSDKPYIV (SEQ ID NO:509), LSDKPYLL (SEQ ID NO:510), LSDKPYLI (SEQ ID NO:511), LSDKPYLV (SEQ ID NO:512), LSDKPYVL (SEQ ID NO:513), LSDKPYVI (SEQ ID NO:514), LSDKPYVV (SEQ ID NO:515), LSDKPWIL (SEQ ID NO:516), LSDKPWII (SEQ ID NO:517), LSDKPWIV (SEQ ID NO:518), LSDKPWLL (SEQ ID NO:519), LSDKPWLI (SEQ ID NO:520), LSDKPWLV (SEQ ID NO:521), LSDKPWVL (SEQ ID NO:522), LSDKPWVI (SEQ ID NO:523), LSDKPWVV (SEQ ID NO:524), LSDRPYIL (SEQ ID NO:525), LSDRPYII (SEQ ID NO:526), LSDRPYIV (SEQ ID NO:527), LSDRPYLL (SEQ ID NO:528), LSDRPYLI (SEQ ID NO:529), LSDRPYLV (SEQ ID NO:530), LSDRPYVL (SEQ ID NO:531), LSDRPYVI (SEQ ID NO:532), LSDRPYVV (SEQ ID NO:533), LSDRPWIL (SEQ ID NO:534), LSDRPWII (SEQ ID NO:535), LSDRPWIV (SEQ ID NO:536), LSDRPWLL (SEQ ID NO:537), LSDRPWLI (SEQ ID NO:538), LSDRPWLV (SEQ ID NO:539), LSDRPWVL (SEQ ID NO:540), LSDRPWVI (SEQ ID NO:541), LSDRPWVV (SEQ ID NO:542), LSEKPYIL (SEQ ID NO:543), LSEKPYII (SEQ ID NO:544), LSEKPYIV (SEQ ID NO:545), LSEKPYLL (SEQ ID NO:546), LSEKPYLI (SEQ ID NO:547), LSEKPYLV (SEQ ID NO:548), LSEKPYVL (SEQ ID NO:549), LSEKPYVI (SEQ ID NO:550), LSEKPYVV (SEQ ID NO:551), LSEKPWIL (SEQ ID NO:552), LSEKPWII (SEQ ID NO:553), LSEKPWIV (SEQ ID NO:554), LSEKPWLL (SEQ ID NO:555), LSEKPWLI (SEQ ID NO:556), LSEKPWLV (SEQ ID NO:557), LSEKPWVL (SEQ ID NO:558), LSEKPWVI (SEQ ID NO:559), LSEKPWVV (SEQ ID NO:560), LSERPYIL (SEQ ID NO:561), LSERPYII (SEQ ID NO:562), LSERPYIV (SEQ ID NO:563), LSERPYLL (SEQ ID NO:564), LSERPYLI (SEQ ID NO:565), LSERPYLV (SEQ ID NO:566), LSERPYVL (SEQ ID NO:567), LSERPYVI (SEQ ID NO:568), LSERPYVV (SEQ ID NO:569), LSERPWIL (SEQ ID NO:570), LSERPWII (SEQ ID NO:571), LSERPWIV (SEQ ID NO:572), LSERPWLL (SEQ ID NO:573), LSERPWLI (SEQ ID NO:574), LSERPWLV (SEQ ID NO:575), LSERPWVL (SEQ ID NO:576), LSERPWVI (SEQ ID NO:577), LSERPWVV (SEQ ID NO:578), LTDKPYIL (SEQ ID NO:579), LTDKPYII (SEQ ID NO:580), LTDKPYIV (SEQ ID NO:581), LTDKPYLL (SEQ ID NO:582), LTDKPYLI (SEQ ID NO:583), LTDKPYLV (SEQ ID NO:584), LTDKPYVL (SEQ ID NO:585), LTDKPYVI (SEQ ID NO:586), LTDKPYVV (SEQ ID NO:587), LTDKPWIL (SEQ ID NO:588), LTDKPWII (SEQ ID NO:589), LTDKPWIV (SEQ ID NO:590), LTDKPWLL (SEQ ID NO:591), LTDKPWLI (SEQ ID NO:592), LTDKPWLV (SEQ ID NO:593), LTDKPWVL (SEQ ID NO:594), LTDKPWVI (SEQ ID NO:595), LTDKPWVV (SEQ ID NO:596), LTDRPYIL (SEQ ID NO:597), LTDRPYII (SEQ ID NO:598), LTDRPYIV (SEQ ID NO:599), LTDRPYLL (SEQ ID NO:600), LTDRPYLI (SEQ ID NO:601), LTDRPYLV (SEQ ID NO:602), LTDRPYVL (SEQ ID NO:603), LTDRPYVI (SEQ ID NO:604), LTDRPYVV (SEQ ID NO:605), LTDRPWIL (SEQ ID NO:606), LTDRPWII (SEQ ID NO:607), LTDRPWIV (SEQ ID NO:608), LTDRPWLL (SEQ ID NO:609), LTDRPWLI (SEQ ID NO:610), LTDRPWLV (SEQ ID NO:611), LTDRPWVL (SEQ ID NO:612), LTDRPWVI (SEQ ID NO:613), LTDRPWVV (SEQ ID NO:614), LTEKPYIL (SEQ ID NO:615), LTEKPYII (SEQ ID NO:616), LTEKPYIV (SEQ ID NO:617), LTEKPYLL (SEQ ID NO:618), LTEKPYLI (SEQ ID NO:619), LTEKPYLV (SEQ ID NO:620), LTEKPYVL (SEQ ID NO:621), LTEKPYVI (SEQ ID NO:622), LTEKPYVV (SEQ ID NO:623), LTEKPWIL (SEQ ID NO:624), LTEKPWII (SEQ ID NO:625), LTEKPWIV (SEQ ID NO:626), LTEKPWLL (SEQ ID NO:627), LTEKPWLI (SEQ ID NO:628), LTEKPWLV (SEQ ID NO:629), LTEKPWVL (SEQ ID NO:630), LTEKPWVI (SEQ ID NO:631), LTEKPWVV (SEQ ID NO:632), LTERPYIL (SEQ ID NO:633), LTERPYII (SEQ ID NO:634), LTERPYIV (SEQ ID NO:635), LTERPYLL (SEQ ID NO:636), LTERPYLI (SEQ ID NO:637), LTERPYLV (SEQ ID NO:638), LTERPYVL (SEQ ID NO:639), LTERPYVI (SEQ ID NO:640), LTERPYVV (SEQ ID NO:641), LTERPWIL (SEQ ID NO:642), LTERPWII (SEQ ID NO:643), LTERPWIV (SEQ ID NO:644), LTERPWLL (SEQ ID NO:645), LTERPWLI (SEQ ID NO:646), LTERPWLV (SEQ ID NO:647), LTERPWVL (SEQ ID NO:648), LTERPWVI (SEQ ID NO:649), LTERPWVV (SEQ ID NO:650), ISDKPYIL (SEQ ID NO:651), ISDKPYII (SEQ ID NO:652), ISDKPYIV (SEQ ID NO:653), ISDKPYLL (SEQ ID NO:654), ISDKPYLI (SEQ ID NO:655), ISDKPYLV (SEQ ID NO:656), ISDKPYVL (SEQ ID NO:657), ISDKPYVI (SEQ ID NO:658), ISDKPYVV (SEQ ID NO:659), ISDKPWIL (SEQ ID NO:660), ISDKPWII (SEQ ID NO:661), ISDKPWIV (SEQ ID NO:662), ISDKPWLL (SEQ ID NO:663), ISDKPWLI (SEQ ID NO:664), ISDKPWLV (SEQ ID NO:665), ISDKPWVL (SEQ ID NO:666), ISDKPWVI (SEQ ID NO:667), ISDKPWVV (SEQ ID NO:668), ISDRPYIL (SEQ ID NO:669), ISDRPYII (SEQ ID NO:670), ISDRPYIV (SEQ ID NO:671), ISDRPYLL (SEQ ID NO:672), ISDRPYLI (SEQ ID NO:673), ISDRPYLV (SEQ ID NO:674), ISDRPYVL (SEQ ID NO:675), ISDRPYVI (SEQ ID NO:676), ISDRPYVV (SEQ ID NO:677), ISDRPWIL (SEQ ID NO:678), ISDRPWII (SEQ ID NO:679), ISDRPWIV (SEQ ID NO:680), ISDRPWLL (SEQ ID NO:681), ISDRPWLI (SEQ ID NO:682), ISDRPWLV (SEQ ID NO:683), ISDRPWVL (SEQ ID NO:684), ISDRPWVI (SEQ ID NO:685), ISDRPWVV (SEQ ID NO:686), ISEKPYIL (SEQ ID NO:687), ISEKPYII (SEQ ID NO:688), ISEKPYIV (SEQ ID NO:689), ISEKPYLL (SEQ ID NO:690), ISEKPYLI (SEQ ID NO:691), ISEKPYLV (SEQ ID NO:692), ISEKPYVL (SEQ ID NO:693), ISEKPYVI (SEQ ID NO:694), ISEKPYVV (SEQ ID NO:695), ISEKPWIL (SEQ ID NO:696), ISEKPWII (SEQ ID NO:697), ISEKPWIV (SEQ ID NO:698), ISEKPWLL (SEQ ID NO:699), ISEKPWLI (SEQ ID NO:700), ISEKPWLV (SEQ ID NO:701), ISEKPWVL (SEQ ID NO:702), ISEKPWVI (SEQ ID NO:703), ISEKPWVV (SEQ ID NO:704), ISERPYIL (SEQ ID NO:705), ISERPYII (SEQ ID NO:706), ISERPYIV (SEQ ID NO:707), ISERPYLL (SEQ ID NO:708), ISERPYLI (SEQ ID NO:709), ISERPYLV (SEQ ID NO:710), ISERPYVL (SEQ ID NO:711), ISERPYVI (SEQ ID NO:712), ISERPYVV (SEQ ID NO:713), ISERPWIL (SEQ ID NO:714), ISERPWII (SEQ ID NO:715), ISERPWIV (SEQ ID NO:716), ISERPWLL (SEQ ID NO:717), ISERPWLI (SEQ ID NO:718), ISERPWLV (SEQ ID NO:719), ISERPWVL (SEQ ID NO:720), ISERPWVI (SEQ ID NO:721), ISERPWVV (SEQ ID NO:722), ITDKPYIL (SEQ ID NO:723), ITDKPYII (SEQ ID NO:724), ITDKPYIV (SEQ ID NO:725), ITDKPYLL (SEQ ID NO:726), ITDKPYLI (SEQ ID NO:727), ITDKPYLV (SEQ ID NO:728), ITDKPYVL (SEQ ID NO:729), ITDKPYVI (SEQ ID NO:730), ITDKPYVV (SEQ ID NO:731), ITDKPWIL (SEQ ID NO:732), ITDKPWII (SEQ ID NO:733), ITDKPWIV (SEQ ID NO:734), ITDKPWLL (SEQ ID NO:735), ITDKPWLI (SEQ ID NO:736), ITDKPWLV (SEQ ID NO:737), ITDKPWVL (SEQ ID NO:738), ITDKPWVI (SEQ ID NO:739), ITDKPWVV (SEQ ID NO:740), ITDRPYIL (SEQ ID NO:741), ITDRPYII (SEQ ID NO:742), ITDRPYIV (SEQ ID NO:743), ITDRPYLL (SEQ ID NO:744), ITDRPYLI (SEQ ID NO:745), ITDRPYLV (SEQ ID NO:746), ITDRPYVL (SEQ ID NO:747), ITDRPYVI (SEQ ID NO:748), ITDRPYVV (SEQ ID NO:749), ITDRPWIL (SEQ ID NO:750), ITDRPWII (SEQ ID NO:751), ITDRPWIV (SEQ ID NO:752), ITDRPWLL (SEQ ID NO:753), ITDRPWLI (SEQ ID NO:754), ITDRPWLV (SEQ ID NO:755), ITDRPWVL (SEQ ID NO:756), ITDRPWVI (SEQ ID NO:757), ITDRPWVV (SEQ ID NO:758), ITEKPYIL (SEQ ID NO:759), ITEKPYII (SEQ ID NO:760), ITEKPYIV (SEQ ID NO:761), ITEKPYLL (SEQ ID NO:762), ITEKPYLI (SEQ ID NO:763), ITEKPYLV (SEQ ID NO:764), ITEKPYVL (SEQ ID NO:765), ITEKPYVI (SEQ ID NO:766), ITEKPYVV (SEQ ID NO:767), ITEKPWIL (SEQ ID NO:768), ITEKPWII (SEQ ID NO:769), ITEKPWIV (SEQ ID NO:770), ITEKPWLL (SEQ ID NO:771), ITEKPWLI (SEQ ID NO:772), ITEKPWLV (SEQ ID NO:773), ITEKPWVL (SEQ ID NO:774), ITEKPWVI (SEQ ID NO:775), ITEKPWVV (SEQ ID NO:776), ITERPYIL (SEQ ID NO:777), ITERPYII (SEQ ID NO:778), ITERPYIV (SEQ ID NO:779), ITERPYLL (SEQ ID NO:780), ITERPYLI (SEQ ID NO:781), ITERPYLV (SEQ ID NO:782), ITERPYVL (SEQ ID NO:783), ITERPYVI (SEQ ID NO:784), ITERPYVV (SEQ ID NO:785), ITERPWIL (SEQ ID NO:786), ITERPWII (SEQ ID NO:787), ITERPWIV (SEQ ID NO:788), ITERPWLL (SEQ ID NO:789), ITERPWLI (SEQ ID NO:790), ITERPWLV (SEQ ID NO:791), ITERPWVL (SEQ ID NO:792), ITERPWVI (SEQ ID NO:793), ITERPWVV (SEQ ID NO:794), VSDKPYIL (SEQ ID NO:795), VSDKPYII (SEQ ID NO:796), VSDKPYIV (SEQ ID NO:797), VSDKPYLL (SEQ ID NO:798), VSDKPYLI (SEQ ID NO:799), VSDKPYLV (SEQ ID NO:800), VSDKPYVL (SEQ ID NO:801), VSDKPYVI (SEQ ID NO:802), VSDKPYVV (SEQ ID NO:803), VSDKPWIL (SEQ ID NO:804), VSDKPWII (SEQ ID NO:805), VSDKPWIV (SEQ ID NO:806), VSDKPWLL (SEQ ID NO:807), VSDKPWLI (SEQ ID NO:808), VSDKPWLV (SEQ ID NO:809), VSDKPWVL (SEQ ID NO:810), VSDKPWVI (SEQ ID NO:811), VSDKPWVV (SEQ ID NO:812), VSDRPYIL (SEQ ID NO:813), VSDRPYII (SEQ ID NO:814), VSDRPYIV (SEQ ID NO:815), VSDRPYLL (SEQ ID NO:816), VSDRPYLI (SEQ ID NO:817), VSDRPYLV (SEQ ID NO:818), VSDRPYVL (SEQ ID NO:819), VSDRPYVI (SEQ ID NO:820), VSDRPYVV (SEQ ID NO:821), VSDRPWIL (SEQ ID NO:822), VSDRPWII (SEQ ID NO:823), VSDRPWIV (SEQ ID NO:824), VSDRPWLL (SEQ ID NO:825), VSDRPWLI (SEQ ID NO:826), VSDRPWLV (SEQ ID NO:827), VSDRPWVL (SEQ ID NO:828), VSDRPWVI (SEQ ID NO:829), VSDRPWVV (SEQ ID NO:830), VSEKPYIL (SEQ ID NO:831), VSEKPYII (SEQ ID NO:832), VSEKPYIV (SEQ ID NO:833), VSEKPYLL (SEQ ID NO:834), VSEKPYLI (SEQ ID NO:835), VSEKPYLV (SEQ ID NO:836), VSEKPYVL (SEQ ID NO:837), VSEKPYVI (SEQ ID NO:838), VSEKPYVV (SEQ ID NO:839), VSEKPWIL (SEQ ID NO:840), VSEKPWII (SEQ ID NO:841), VSEKPWIV (SEQ ID NO:842), VSEKPWLL (SEQ ID NO:843), VSEKPWLI (SEQ ID NO:844), VSEKPWLV (SEQ ID NO:845), VSEKPWVL (SEQ ID NO:846), VSEKPWVI (SEQ ID NO:847), VSEKPWVV (SEQ ID NO:848), VSERPYIL (SEQ ID NO:849), VSERPYII (SEQ ID NO:850), VSERPYIV (SEQ ID NO:851), VSERPYLL (SEQ ID NO:852), VSERPYLI (SEQ ID NO:853), VSERPYLV (SEQ ID NO:854), VSERPYVL (SEQ ID NO:855), VSERPYVI (SEQ ID NO:856), VSERPYVV (SEQ ID NO:857), VSERPWIL (SEQ ID NO:858), VSERPWII (SEQ ID NO:859), VSERPWIV (SEQ ID NO:860), VSERPWLL (SEQ ID NO:861), VSERPWLI (SEQ ID NO:862), VSERPWLV (SEQ ID NO:863), VSERPWVL (SEQ ID NO:864), VSERPWVI (SEQ ID NO:865), VSERPWVV (SEQ ID NO:866), VTDKPYIL (SEQ ID NO:867), VTDKPYII (SEQ ID NO:868), VTDKPYIV (SEQ ID NO:869), VTDKPYLL (SEQ ID NO:870), VTDKPYLI (SEQ ID NO:871), VTDKPYLV (SEQ ID NO:872), VTDKPYVL (SEQ ID NO:873), VTDKPYVI (SEQ ID NO:874), VTDKPYVV (SEQ ID NO:875), VTDKPWIL (SEQ ID NO:876), VTDKPWII (SEQ ID NO:877), VTDKPWIV (SEQ ID NO:878), VTDKPWLL (SEQ ID NO:879), VTDKPWLI (SEQ ID NO:880), VTDKPWLV (SEQ ID NO:881), VTDKPWVL (SEQ ID NO:882), VTDKPWVI (SEQ ID NO:883), VTDKPWVV (SEQ ID NO:884), VTDRPYIL (SEQ ID NO:885), VTDRPYII (SEQ ID NO:886), VTDRPYIV (SEQ ID NO:887), VTDRPYLL (SEQ ID NO:888), VTDRPYLI (SEQ ID NO:889), VTDRPYLV (SEQ ID NO:890), VTDRPYVL (SEQ ID NO:891), VTDRPYVI (SEQ ID NO:892), VTDRPYVV (SEQ ID NO:893), VTDRPWIL (SEQ ID NO:894), VTDRPWII (SEQ ID NO:895), VTDRPWIV (SEQ ID NO:896), VTDRPWLL (SEQ ID NO:897), VTDRPWLI (SEQ ID NO:898), VTDRPWLV (SEQ ID NO:899), VTDRPWVL (SEQ ID NO:900), VTDRPWVI (SEQ ID NO:901), VTDRPWVV (SEQ ID NO:902), VTEKPYIL (SEQ ID NO:903), VTEKPYII (SEQ ID NO:904), VTEKPYIV (SEQ ID NO:905), VTEKPYLL (SEQ ID NO:906), VTEKPYLI (SEQ ID NO:907), VTEKPYLV (SEQ ID NO:908), VTEKPYVL (SEQ ID NO:909), VTEKPYVI (SEQ ID NO:910), VTEKPYVV (SEQ ID NO:911), VTEKPWIL (SEQ ID NO:912), VTEKPWII (SEQ ID NO:913), VTEKPWIV (SEQ ID NO:914), VTEKPWLL (SEQ ID NO:915), VTEKPWLI (SEQ ID NO:916), VTEKPWLV (SEQ ID NO:917), VTEKPWVL (SEQ ID NO:918), VTEKPWVI (SEQ ID NO:919), VTEKPWVV (SEQ ID NO:920), VTERPYIL (SEQ ID NO:921), VTERPYII (SEQ ID NO:922), VTERPYIV (SEQ ID NO:923), VTERPYLL (SEQ ID NO:924), VTERPYLI (SEQ ID NO:925), VTERPYLV (SEQ ID NO:926), VTERPYVL (SEQ ID NO:927), VTERPYVI (SEQ ID NO:928), VTERPYVV (SEQ ID NO:929), VTERPWIL (SEQ ID NO:930), VTERPWII (SEQ ID NO:931), VTERPWIV (SEQ ID NO:932), VTERPWLL (SEQ ID NO:933), VTERPWLI (SEQ ID NO:934), VTERPWLV (SEQ ID NO:935), VTERPWVL (SEQ ID NO:936), VTERPWVI (SEQ ID NO:937), and VTERPWVV (SEQ ID NO:938).

In some specific embodiments, the polypeptide for use in compositions according to the present invention consists of or comprises an amino acid sequence derived from Alpha-actinin-1, such as a sequence selected from ASDKPYIL, AGDKNYIL, AGDKNYIT, AGDKSYIT, ADGKPYIV, and AEDKDFIT.

In some specific embodiments, the polypeptide for use in compositions according to the present invention consists of or comprises an amino acid sequence derived from Alpha-actinin-2, such as a sequence selected from ASDKPYIL, AADKPYIL, AGDKNYIT, ATDKPYIL, AGDKPYIT, ASEKPYIL, ADGKPYVT, AGDKPYIL, ASDKPNIL, ASDKPYIT, AADKPFIL, ASDKAYIT, AGDKAYIT, ANGKPFIT, and AGDKNFIT.

In some specific embodiments, the polypeptide for use in compositions according to the present invention consists of or comprises an amino acid sequence derived from Alpha-actinin-3, such as a sequence selected from ASDKPYIL, AADKPYIL, ASDKAYIT, ASDKSYIT, ASDKTYIT, ASDKNYIT, AGDKNYIL, AGDKSYIT, AGDKNYIT, AGDKKYIT, and AGDKNYIS.

In some specific embodiments, the polypeptide for use in compositions according to the present invention consists of or comprises an amino acid sequence derived from Alpha-actinin-4, such as a sequence selected from ASDKPYIL, AGDKPYIL, AADKNYIT, AGDKNYIM, AGDKNYIT, AADKNFIM, AADKNFIT, AGDKGIRS, and AGDKNFIT.

The present invention further relates to compositions comprising the polypeptides of the invention. In some embodiments the compositions of the invention is capable of promoting satiety or for reducing feed intake in a subject upon consumption.

In some embodiments in the compositions for use in compositions according to the present invention the amount of said polypeptide in the composition is less than about 10 g, such as less than 9 g, 8 g, 7 g, 6 g, 5 g, 4 g, 3 g, 2 g, 1 g, 900 mg, 800 mg, 700 mg, 600 mg, 500 mg, 400 mg, 300 mg, 200 mg, 150 mg, 100 mg, 90 mg, 80 mg, 70 mg, 60 mg, 50 mg, 40 mg, 30 mg, 25 mg, 20 mg, 15 mg, 10 mg, or 5 mg.

In some embodiments in the compositions of the invention the amount of said polypeptide in the composition is at least about 5 mg, such as at least about 10 mg, 15 mg, 20 mg, 25 mg, 30 mg, 40 mg, 50 mg, 60 mg, 70 mg, 80 mg, 90 mg, 100 mg, 150 mg, 200 mg, 300 mg, 400 mg, 500 mg, 600 mg, 700 mg, 800 mg, 900 mg, 1 g, 2 g, 3 g, 4 g, 5 g, 6 g, 7 g, 8 g, 9 g, or 10 g.

In some embodiments in the compositions of the invention the energy content derived through the process of cellular respiration is less than 50 kilojoules (kJ), such as less than 40 kJ, such as less than 30 kJ, such as less than 20 kJ, such as less than 10 kJ, such as less than 5000 Joules (J), such as less than 1000 J, such as less than 900 J, such as less than 800 J, such as less than 700 J, such as less than 600 J, such as less than 500 J, such as less than 400 J, such as less than 300 J, such as less than 200 J, such as less than 100 J, such as less than 50 J.

In some embodiments the compositions of the invention is a food composition.

In some embodiments the compositions of the invention is a fermented composition.

In some embodiments the compositions of the invention is a dairy product.

In some embodiments the compositions of the invention is a pharmaceutical composition.

In some embodiments the compositions of the invention is a nutritional composition.

In some embodiments the compositions of the invention is an oral dosage form. In some embodiments the oral dosage form is selected from the group comprising tablets, capsules, caplets, slurries, sachets, suspensions, chewing gum, and powder formulation that may be dissolved in a liquid. In some embodiments the oral dosage form is a suspension. In some embodiments the oral dosage form is a powder formulation that may be dissolved in a liquid. In some embodiments the liquid is water, milk, juice, or yogurt.

Another aspect of the invention related to a composition comprising i) a gastrointestinal peptide hormone and ii) a protease inhibitor, such as potato proteinase inhibitor II (PI2), such as PI2 derived from a potato protein extract.

In some embodiments the gastrointestinal peptide hormone is any one peptide described above, or a peptide selected from the list consisting of Cholecystokinin (CCK), Gastrin, Secretin, Vasoactive Intestinal Peptide (VIP), Glucose-dependent insulinotropic peptide (GIP), Glucagon-like Peptide 1 and 2 (GLP-1 and -2), Bombesin, Chromogranin A, Glucagon, Insulin, Leptin, Neuropeptide Y, Neurotensin, Neuromedin, Pancreatic Polypeptide, PYY, Amylin, Oxyntomodulin, Xexin, Motilin, Grehlin, and Somatostatin, and bioactive analogues or variants of any one of these peptide hormones.

In some embodiments the potato proteinase inhibitor II (PI2) is derived from a potato protein extract.

In some embodiments PI2 is derived from a protein extract from potato, such as a side-stream from the production of potato starch.

In some embodiments PI2 is a potato protein extract present in said composition in an amount higher than 2% of total protein, such as higher than 3, 4, 5, 7, 8, 9, 10, 15, 20, 25, 30, 35, 40, 45, or 50% of total protein.

In some embodiments PI2 is from a potato protein extract with a content of trypsin and chymotrypsin inhibitors higher than 10% of total protein, such as higher than 20, 25, 30, 35, 40, 45, or 50% of total protein of said potato protein extract.

In some embodiments PI2 is from a potato protein extract with a content of carboxypeptidase inhibitors higher than 1, such 2, 3, 4, 5, 6, 7, 8, 9, 10, 12, 14, 16, 18, 20, 25, 30, or 35% of total protein of said potato protein extract.

In some embodiments PI2 is prepared from heat treated or ethanol extracted potato protein extract.

In some embodiments PI2 is derived from potato protein extract obtained by filtration (>10 kDa) of potato juice from starch processing prior to heat-treatment.

In some embodiments PI2 is derived from potato protein extract prepared from both heat-treated potato protein extract (PE, ‘Protamylasse’) and potato protein isolate (PPI) obtained by filtration (>10 kDa) of potato juice from starch processing prior to heat-treatment.

In some embodiments PI2 is present in said composition in an amount of at least about 5 mg, such as 10, 15, 20, 25, 30, 35, 40, 45, 50, 55, 60, 65, 70, 75, 80, 85, 90, 95, or 100 mg.

In some embodiments the composition according to the invention is capable of promoting satiety or for reducing feed intake in a subject upon consumption.

In some embodiments the amount of said polypeptide in the composition is less than about 10 g, such as less than 9 g, 8 g, 7 g, 6 g, 5 g, 4 g, 3 g, 2 g, 1 g, 900 mg, 800 mg, 700 mg, 600 mg, 500 mg, 400 mg, 300 mg, 200 mg, 150 mg, 100 mg, 90 mg, 80 mg, 70 mg, 60 mg, 50 mg, 40 mg, 30 mg, 25 mg, 20 mg, 15 mg, 10 mg, or 5 mg.

In some embodiments the amount of said polypeptide in the composition is at least about 5 mg, such as at least about 10 mg, 15 mg, 20 mg, 25 mg, 30 mg, 40 mg, 50 mg, 60 mg, 70 mg, 80 mg, 90 mg, 100 mg, 150 mg, 200 mg, 300 mg, 400 mg, 500 mg, 600 mg, 700 mg, 800 mg, 900 mg, 1 g, 2 g, 3 g, 4 g, 5 g, 6 g, 7 g, 8 g, 9 g, or 10 g.

In some embodiments in the composition according to the invention the energy content derived through the process of cellular respiration is less than 50 kilojoules (kJ), such as less than 40 kJ, such as less than 30 kJ, such as less than 20 kJ, such as less than 10 kJ, such as less than 5000 Joules (J), such as less than 1000 J, such as less than 900 J, such as less than 800 J, such as less than 700 J, such as less than 600 J, such as less than 500 J, such as less than 400 J, such as less than 300 J, such as less than 200 J, such as less than 100 J, such as less than 50 J.

In some embodiments the composition is a food composition.

In some embodiments the composition is a fermented composition.

In some embodiments the composition is a dairy product.

In some embodiments the composition is a pharmaceutical composition.

In some embodiments the composition is a nutritional composition.

In some embodiments the composition is an oral dosage form.

In some embodiments in the composition according to the invention the oral dosage form is selected from the group comprising tablets, capsules, caplets, slurries, sachets, suspensions, chewing gum, and powder formulation that may be dissolved in a liquid.

In some embodiments in the composition according to the invention the oral dosage form is a suspension.

In some embodiments in the composition according to the invention the oral dosage form is a powder formulation that may be dissolved in a liquid. In some embodiments the liquid is water, milk, juice, or yogurt.

In some embodiments the composition according to the invention further comprises a protein substrate for any one protease, such as an endoproteinase, such as trypsin or chymotrypsin, and an exoproteinase, such as carboxypeptidases, such as Carboxypeptidase A and B.

In some embodiments the composition according to the invention further comprises protein substrate for any one protease, which is as a crude protein hydrolysate, such as present in a ratio relative to said polypeptide higher than 1:1, such as higher than 2:1, 3:1, 4:1, 5:1, 6:1, 7:1, 8:1, 9:1, 10:1, 15:1, 20:1, 25:1, 30:1, 35:1, 40:1, 50:1, 100:1, 1000:1, 10000:1, 100000:1, 1000000:1, 10000000:1, 100000000:1, or 1000000000:1 as measured in weight of protein. In some embodiments the protein substrate is derived from an animal, such as meat, such as bovine heart, such as from blood or plasma, or from a milk product, such as whey, from a plant, such as cereals, such as wheat, legumes, soy, rice, nuts, seeds, from micro or macroalgae, such as Spirulina, or from a microorganism, such as bacteria, such as Bacillus sp. Or from fungi, such as yeast, such as Saccharomyces sp.

### Example 1

Assays:

Ca²⁺ Flux Assay/Calcium Response Assay:

Elevation of intracellular calcium level was measured using the fluorescent calcium chelating dye Fluo-4 AM (ThermoFischer Scientific, Denmark). Briefly, cells were grown as a monolayer in 96-well tissue culture plates (Sarstedt, Germany) to near confluence in appropriate growth medium as described in the cell culture section. Prior to the start of the assay, the cells were incubated with 1.5 μM Fluo-4 AM in complete culture media mixed 1:1 with Hank's balanced salt solution (HBSS, ThermoFischer Scientific, Denmark) containing 25 mM HEPES (pH 7.4), 1% BSA (Sigma-Aldrich, Denmark), 2% ink (Soluro GMBH, Germany), 0.01% Pluronic F-127 (Sigma-Aldrich, Denmark) and 1 mM Probenecide (Sigma-Aldrich) for 60 minutes at 37° C.

All test compounds were dissolved in water, and then diluted in 1×HBSS containing 25 mM HEPES (pH 7.4), 1% BSA and 2% ink. Without any removal of excess Fluo-4 AM, test compounds were added directly into the wells and fluorescence were measured using instrument settings for excitation at 488 nm and emission at 525 nm in a microtiter plate reader (SpectraMax M5, Molecular Devices, USA).

Cell Culture:

Cell culture media, Dulbecco's phosphate-buffered saline, pH 7.4 (DPBS), glutamine, trypsin-EDTA and antibiotics were obtained from ThermoFischer Scientific (Denmark). Fetal bovine serum and all other chemicals were purchased from Sigma-Aldrich (Denmark), unless otherwise stated.

Murine intestinal enteroendocrine L-cell lines that expresses the proglucagon gene and secretes GLP-1 in vitro were used. Cells were grown in DMEM containing 1 g/L D-glucose, 10% fetal bovine serum, 2 mM glutamine, 1% penicillin/streptomycin/neomycin and cultured in a humidified incubator in 95% air and 5% CO2 at 37° C.

Other murine intestinal cell lines were cultured in Dulbecco's modified Eagle's medium (DMEM) supplemented with 2 mM glutamine, 2.5 g/L glucose, 20 mM HEPES, 60 nM sodium selenite, 5 μg/ml transferrin, 5 μg/ml insulin, 50 nM dexamethasone, 10 nM EGF, 1 nM triiodothyronine, 2% fetal bovine serum and 1% penicillin/streptomycin/neomycin at 37° C. in 5% CO2-95% air atmosphere.

Human intestinal cell lines were cultured in McCoy's modified 5A medium supplemented with 10% fetal bovine serum and 1% penicillin/streptomycin/neomycin at 37° C. and 5% CO2 in a humidified incubator.

Cells were routinely sub-cultivated 1:3 and given new media every second day.

Determination of GLP-1 Levels:

GLP-1 levels were determined using a sandwich enzyme-linked immunoabsorbant assay (ELISA). The primary antibody to GLP-1 [2.5 μg/ml mouse monoclonal (HYB 147-06) in 0.05M bicarbonate/carbonate buffer; BioPorto Diagnostics A/S, Gentofte, Denmark) was coated on a flat-bottom 96-well plate (Sarstedt, Nümbrecht, Germany) for at least 24 hours at 4° C. This primary antibody is specific for the amidated C-terminus of the peptide and reacts with GLP-1 (7-36), GLP-1 (9-36) and GLP-1 (1-36), but not with GLP-1 (7-37). After blocking the plate using a PBS buffer containing 4% w/v BSA (Sigma-Aldrich, Denmark) and 0.1% v/v Tween 20 (Sigma-Aldrich) for 1 hour at room temperature, the plate was washed four times with PBS buffer containing 0.1% v/v Tween 20. A standard curve with GLP-1 peptide [human GLP-1 (7-36), Sigma-Aldrich, Denmark) concentrations ranging from 0 pg/ml to 1000 pg/ml was prepared in PBS buffer containing 0.5% BSA and 0.05% Tween 20, and samples were diluted if necessary. Samples and standards were added to the microtiter plate and incubated with the primary antibody for two hours at room temperature. Subsequently, the plate was washed four times, and the wells were incubated with a secondary biotinylated antibody to GLP-1 [1 μg/ml; mouse monoclonal (ABS 033-01), BioPorto Diagnostics A/S, Gentofte, Denmark) for two hours at room temperature. After another washing step, samples were incubated with streptavidin-horseradish peroxidase (1:200, Dako A/S, Denmark) for 45 minutes followed by an incubation with TMB solution (containing 3,3′,5,′5-tetramethylbenzidine and H2O2, SMS-gruppen, Denmark). The reaction was stopped by adding H2SO4 (0.2M), and the absorbance of the yellow end product was measured at 450 nm on a microtiter plate spectrophotometer (Spectra Max M5, Molecular Devices, USA). The concentrations of the samples were determined by interpolation to the concentrations of the standard solutions.

Cells (˜5×10\hat{ }5 per sample) were incubated for up to 90 min in Dulbeccos Modified Eagle Medium (DMEM) containing 5.56 mM glucose in absence or presence of different amounts (weight/volume) of protein hydrolysate (pig heart). Supernatant was filtered through 0.45 micron filters and assayed for content of GLP-1 as described in ELISA protocol. Data are mean+SEM from quadruplicate samples.

### Preparation of Bioactive Peptides by Enzymatic Digestion of Meat

Minced meat is diluted 1-10 times with distilled water, adjusted to pH 1-3 with hydrochloric acid, and incubated with 0.01-10% pepsin (w/w) at 4-40° C. for ½-12 h with adequate mixing. Insoluble material is removed by centrifugation at 100-1000×g for 3-30 min, and supernatant is neutralized with NaOH. Using sterile conditions, low molecular weight peptides in supernatant are recovered by tangential ultrafiltration at 4-40° C. for ½-12 h, and excess water is evaporated at 25-50° C. for up to 12 h. The concentrated dialysate is tested for bioactivity with cells and used for further purification by HPLC.

Purification and Identification of Bioactive Peptide ASDKPYIL

Upconcentrated dialysates were fractionated on preparative C18 columns using buffer B: 20 mM phosphate buffer pH 8.25/10% ACN and a gradient of 0-40% in buffer A: 60% ACN in same buffer. Fractions were tested for bioactivity and further purified by isocratic elution using EVO C18 columns with 4.5% ACN in 0.1% FA isocratic for 30 min. Fractions were subject to MS characterization, where a dominating peak with m/z 453.75 (+2) was observed. Extracted ions chromatograms show this peak to be present in all active fractions. De novo sequencing of 453.75 peak gives [A]SDKPY[I,L][I,L]. N-terminal A is calculated from parent ion -A7. I and L are not resolved by MS because of equal molecular weights. Search of protein sequences gives only ASDKPYIL as match. ASDKPYIL is only found in alpha-actinin-2, a major muscle protein.

Stability of Peptides Ex Vivo.

Peptides are degraded by proteases in the gastrointestinal tract. However the speed of this degradation depends on the sequence of the peptide. In order to measure stability of the ASDKPYIL peptide series and to compare with e.g. RRKPYIL, 10 or 50 mg (wet weight) of mouse or rat intestinal tissue (distal ileum) was equilibrated in V-bottom 24 well plates in 800 μl HBSS, 25 mM HEPES, pH 7.4 at 37° C. with shaking at 350 rpm. Identical amounts of different peptides (final concentration of 1 μg/ml) were added to the intestinal pieces and incubation continued. At various time points, 100 μl aliquots were removed and diluted into whey protein hydrolysate (final concentration of 10 mg/ml) to non-specifically compete protease activity. Peptide solutions were then diluted and tested for bioactivity (FIG. 8). Peptides incubated under same conditions but in absence of intestine served as controls (no degradation). Determination of EC₅₀ for stimulation of cells allowed calculation of recovered peptide (FIG. 9), assuming simple inactivation by the tissue.

### Example 2

1) Structure-activity relationship and stability (SAR)
a. Extended versions
b. Substituted versions

2) In vivo studies in mice
c. Acute effects on feed intake (satiety)
d. Long-term effects on weight may be determined

Based on structural modelling studies of DC7-2 and NTR-1 interactions, peptides being octapeptides, heptapeptides, hexapeptides, or pentapeptides to exhibit increased potency due to increased binding may be predicted.

Comparison with SAR studies using synthetic peptides, peptides with increased potency and stability may be both predicted and observed.

Assays:

Synthetic Peptides:

Based on the sequence of the natural hormone Neurotensin (QLYENKPRRPYIL), the bioactive Neurotensin fragment NT(8-13)(RRPYIL) and the identified bioactive octapeptide DC7-2 (ASDKPYIL), synthetic peptides with systematic substitutions of N-terminal amino acids of the octapeptide (X-SDKPYIL), the heptapeptide (X-DKPYIL), the hexapeptide (X-KPYIL) and the pentapeptide (X-PYIL) were synthesised using standard techniques (Schafer-N, Denmark). All peptides were dissolved in pure HPLC-grade water and stored at −20° C.

Stability of Peptides:

Concentration Determination:

Protein concentration of synthetic peptides (Schafer-N, Denmark), NT (Sigma-Aldrich, Denmark) and NT (8-13)(Sigma-Aldrich, Denmark) were determined by measuring absorbance at 280 nm in Costar® 96-well UV-transparent plates (Corning, Sigma-Aldrich, Denmark). Each peptide was measured in 4 different concentrations by dilution in Hank's balanced salt solution (HBSS, ThermoFischer Scientific, Denmark) containing 25 mM HEPES (pH 7.4) (Sigma-Aldrich, Denmark). For stability assays, all peptides were diluted to 3×10⁻⁵ M in HBSS; 25 mM HEPES (Ph 7.4) and stored at +4° C.

Intestine Homogenate:

Small intestines from 20 Swiss-Webster males were homogenized in 350 ml Dulbecco's phosphate-buffered saline (PBS) (pH 7.4) (ThermoFischer Scientific, Denmark) with a IKA® basic 18 Ultra-Turrax tissue homogenizer set a speed 5 followed by filtration using 100 μm nylon mesh filter. Protein concentration was 6 mg/ml using the bicinconinic acid assay (ThermoFischer Scientific, Denmark) and bovine serum albumin as standard. The intestine homogenate was diluted 10 times in HBSS containing 25 mM HEPES (pH 7.4), and further diluted 30×, 90×, 270×, 810× or 2430× before incubation with peptides. All solutions were prewarmed to 37° C. before mixing with peptide solutions.

Peptides were incubated at 10⁻⁵ M with dilutions of small intestine homogenate at 37° C. for 90 minutes with shaking. Reactions were stopped by addition of 1 M phosphoric acid (final 0.4 M, pH˜1.2). Each peptide incubation mix was then neutralized with NaOH to pH 7.2-7.4 and immediately tested for activity in intestinal cells. Control for zero degradation, i.e. addition of 1 M phosphoric acid prior to addition of intestine homogenate, was included for each peptide.

Fetal Bovine Serum:

All peptides were incubated at 10⁻⁵ M with Fetal Bovine Serum (FBS; final concentration of 66.7%) (Sigma Aldrich, Denmark) at 37° C. for 3 hours. The peptide degradation was terminated using 1 M phosphoric acid (final 0.4 M, pH˜1.2) and neutralized to pH 7.2-7.4 with NaOH before testing activity in intestinal cells. As for small intestine homogenates, a zero degradation control was included for each peptide.

Kinetic Studies of Selected Peptides:

DC7-2, NT, DKPYIL and NT-(8-13) (final concentration of 10⁻⁶ M) were incubated either with FBS or with 270× diluted intestinal homogenate at 37° C. for various time points with shaking. Degradation was stopped with 1 M phosphoric acid and the samples were subsequently neutralized and immediately tested with intestinal cells as described above. Control for zero degradation was included for each peptide as above.

Study of Hexapeptides:

The 20 hexapeptides with systematic N-terminal substitutions (X-KPYIL) (Schafer-N, Denmark) and NT (8-13) was incubated at 10⁻⁶ M in either FBS for 10 minutes or with 270× diluted intestinal homogenate for 30 minutes at 37° C. with shaking. The degradation was stopped with 1 M phosphoric acid. Peptide solutions were neutralized with NaOH (pH 7.2-7.4), diluted and immediately tested for bioactivity using murine intestinal cells. Determination of EC50 for stimulation of cells allowed calculation of recovered peptide.

Systematic substitutions of N-terminal amino acids in octapeptide ASDKPYIL and their importance for activity and stability. Sequence, activity and stability of DC7-2 is italicized.

| | | | | | Stability |
|---|---|---|---|---|---|
| | | Cell signaling | | Stability | in |
| | | activity | | in serum | intestine |
| Peptide | | (EC50, nM) | | (activity | (activity |
| ID | Sequence | Mean | SEM | remaining)¹⁾ | remaining)²⁾ |
| 36055 | Y SDKPYIL (SEQ ID NO: 939) | 5.92E−09 | 5.53E−10 | 0.0491 | 3.0 |
| 36054 | W SDKPYIL (SEQ ID NO: 940) | 8.87E−09 | 7.94E−10 | 0.0426 | 3.6 |
| 36053 | V SDKPYIL (SEQ ID NO: 795) | 5.15E−09 | 5.32E−10 | 0.0488 | 5.6 |
| 36052 | T SDKPYIL (SEQ ID NO: 941) | 5.63E−09 | 5.67E−10 | 0.0535 | 8.4 |
| 36051 | S SDKPYIL (SEQ ID NO: 942) | 4.27E−09 | 3.92E−10 | 0.0609 | 19.5 |
| 36050 | R SDKPYIL (SEQ ID NO: 943) | 4.01E−09 | 4.43E−10 | 0.0735 | 2.6 |
| 36049 | Q SDKPYIL (SEQ ID NO: 944) | 4.01E−09 | 4.51E−10 | 0.0742 | 8.8 |
| 36048 | P SDKPYIL (SEQ ID NO: 945) | 4.14E−09 | 4.54E−10 | 0.0772 | 4.6 |
| 36047 | N SDKPYIL (SEQ ID NO: 946) | 4.41E−09 | 4.28E−10 | 0.0872 | 3.9 |
| 36046 | M SDKPYIL (SEQ ID NO: 947) | 4.88E−09 | 5.01E−10 | 0.0762 | 3.8 |
| 36045 | L SDKPYIL (SEQ ID NO: 507) | 8.06E−09 | 8.25E−10 | 0.0917 | 7.9 |
| 36044 | K SDKPYIL (SEQ ID NO: 948) | 1.07E−08 | 1.05E−09 | 0.0527 | 6.6 |
| 36043 | I SDKPYIL (SEQ ID NO: 651) | 6.87E−09 | 7.41E−10 | 0.0956 | 6.0 |
| 36042 | H SDKPYIL (SEQ ID NO: 949) | 3.63E−09 | 3.24E−10 | 0.0980 | 6.4 |
| 36041 | G SDKPYIL (SEQ ID NO: 950) | 4.07E−09 | 5.07E−10 | 0.0912 | 10.3 |
| 36040 | F SDKPYIL (SEQ ID NO: 951) | 3.85E−09 | 4.95E−10 | 0.0944 | 3.4 |
| 36039 | E SDKPYIL (SEQ ID NO: 952) | 4.82E−09 | 5.76E−10 | 0.0964 | 23.0 |
| 36038 | D SDKPYIL (SEQ ID NO: 953) | 6.15E−09 | 6.85E−10 | 0.0947 | 29.9 |
| 36037 | C SDKPYIL (SEQ ID NO: 954) | 5.44E−09 | 6.25E−10 | 0.0963 | 14.0 |
| 36036 | A SDKPYIL (SEQ ID NO: 6) | 3.51E−09 | 4.75E−10 | 0.0988 | 6.0 |

Notes for Tables
¹⁾Stability in serum is expressed as fraction of peptide activity left after 10 min of incubation in serum at 37° C. compared with undigested sample as described in Examples.
²⁾Stability in intestine is expressed as % activity left after 30 min incubation in intestine homogenate at 37° C. as described in Examples.

Systematic substitutions of N-terminal amino acid in heptapeptide SDKPYIL and their importance for activity and stability. Sequence, activity and stability of peptide contained in DC7-2 is italicized.

| | | Cell signaling | | | Stability |
|---|---|---|---|---|---|
| | | activity | | Stability | in |
| Peptide | | (EC50, nM) | | in serum | intestine |
| ID | Sequence | Mean | STD | (t½, min) | (t½, min) |
| 36035 | Y DKPYIL (SEQ ID NO: 955) | 6.83E−09 | 5.97E−10 | 0.0531 | 3.2 |
| 36034 | W DKPYIL (SEQ ID NO: 956) | 1.41E−08 | 1.18E−09 | 0.0427 | 6.3 |
| 36033 | V DKPYIL (SEQ ID NO: 957) | 4.94E−09 | 4.75E−10 | 0.0528 | 4.4 |
| 36032 | T DKPYIL (SEQ ID NO: 292) | 5.61E−09 | 5.33E−10 | 0.0593 | 13.3 |
| 36031 | S DKPYIL (SEQ ID NO: 7) | 5.00E−09 | 4.88E−10 | 0.0587 | 10.6 |
| 36030 | R DKPYIL (SEQ ID NO: 958) | 4.68E−09 | 4.77E−10 | 0.0597 | 6.9 |
| 36029 | Q DKPYIL (SEQ ID NO: 959) | 4.97E−09 | 4.86E−10 | 0.0620 | 11.5 |
| 36028 | P DKPYIL (SEQ ID NO: 960) | 4.67E−09 | 4.64E−10 | 0.0558 | 10.9 |
| 36027 | N DKPYIL (SEQ ID NO: 961) | 5.92E−09 | 5.21E−10 | 0.0580 | 40.5 |
| 36026 | M DKPYIL (SEQ ID NO: 962) | 6.08E−09 | 5.69E−10 | 0.0601 | 7.1 |
| 36025 | L DKPYIL (SEQ ID NO: 963) | 6.41E−09 | 9.98E−10 | 0.0969 | 3.9 |
| 36024 | K DKPYIL (SEQ ID NO: 964) | 1.12E−08 | 1.61E−09 | 0.0910 | 6.9 |
| 36023 | I DKPYIL (SEQ ID NO: 965) | 4.77E−09 | 8.27E−10 | 0.0928 | 2.8 |
| 36022 | H DKPYIL (SEQ ID NO: 966) | 2.65E−09 | 3.53E−10 | 0.0932 | 4.7 |
| 36021 | G DKPYIL (SEQ ID NO: 967) | 2.91E−09 | 3.86E−10 | 0.0920 | 4.5 |
| 36020 | F DKPYIL (SEQ ID NO: 968) | 9.52E−09 | 1.38E−09 | 0.0901 | 2.4 |
| 36019 | E DKPYIL (SEQ ID NO: 969) | 3.96E−09 | 6.89E−10 | 0.0846 | 17.2 |
| 36018 | D DKPYIL (SEQ ID NO: 970) | 1.05E−08 | 1.47E−09 | 0.0419 | 44.3 |
| 36017 | C DKPYIL (SEQ ID NO: 971) | 7.72E−09 | 1.17E−O9 | 0.0407 | 9.4 |
| 36016 | A DKPYIL (SEQ ID NO: 972) | 3.52E−09 | 6.43E−10 | 0.0952 | 2.5 |

Systematic substitutions of N-terminal amino acid in hexapeptide DKPYIL and their importance for activity and stability. Sequence, activity and stability of peptide contained in DC7-2 is italicized.

| | | Cell signaling | | | Stability |
|---|---|---|---|---|---|
| | | activity | | Stability | in |
| Peptide | | (EC50, nM) | | in serum | intestine |
| ID | Sequence | Mean | STD | (t½, min) | (t½, min) |
| 35995 | Y KPYIL (SEQ ID NO: 973) | 3.34E−09 | 5.33E−10 | 0.0346 | 0.1 |
| 35994 | W KPYIL (SEQ ID NO: 974) | 5.58E−09 | 8.57E−10 | 0.0334 | 0.2 |
| 35993 | V KPYIL (SEQ ID NO: 975) | 1.17E−09 | 1.73E−10 | 0.0375 | 0.2 |
| 35992 | T KPYIL (SEQ ID NO: 976) | 1.16E−09 | 1.72E−10 | 0.0424 | 0.3 |
| 35991 | S KPYIL (SEQ ID NO: 977) | 1.15E−09 | 1.70E−10 | 0.0477 | 1.3 |
| 35990 | R KPYIL (SEQ ID NO: 150) | 4.40E−10 | 6.59E−11 | 0.0480 | 0.1 |
| 35989 | Q KPYIL (SEQ ID NO: 978) | 3.78E−10 | 5.67E−11 | 0.0495 | 0.8 |
| 35988 | P KPYIL (SEQ ID NO: 979) | 2.32E−10 | 3.53E−11 | 0.0550 | 0.2 |
| 35987 | N KPYIL (SEQ ID NO: 980) | 5.00E−10 | 7.42E−11 | 0.0709 | 1.0 |
| 35986 | M KPYIL (SEQ ID NO: 981) | 3.88E−10 | 5.82E−11 | 0.0504 | 0.1 |
| 35985 | L KPYIL (SEQ ID NO: 982) | 3.30E−10 | 4.60E−11 | 0.0310 | 0.1 |
| 35984 | K KPYIL (SEQ ID NO: 43) | 2.64E−10 | 3.72E−11 | 0.0403 | 0.1 |
| 35983 | I KPYIL (SEQ ID NO: 983) | 2.40E−10 | 3.37E−11 | 0.0315 | 0.0 |
| 35982 | H KPYIL (SEQ ID NO: 984) | 2.71E−10 | 3.82E−11 | 0.0363 | 0.1 |
| 35981 | G KPYIL (SEQ ID NO: 184) | 3.64E−10 | 5.05E−11 | 0.0353 | 0.1 |
| 35980 | F KPYIL (SEQ ID NO: 985) | 3.15E−10 | 4.39E−11 | 0.0365 | 0.1 |
| 35979 | E KPYIL (SEQ ID NO: 114) | 5.65E−10 | 7.80E−11 | 0.0478 | 0.6 |
| 35978 | D KPYIL (SEQ ID NO: 8) | 8.03E−10 | 1.11E−10 | 0.0623 | 2.2 |
| 35977 | C KPYIL (SEQ ID NO: 986) | 1.11E−09 | 1.53E−10 | 0.0477 | 2.6 |
| 35976 | A KPYIL (SEQ ID NO: 987) | 2.51E−10 | 3.50E−11 | 0.0454 | 0.1 |

Systematic substitutions of N-terminal amino acid in pentapeptide KPYIL and their importance for activity and stability. Sequence, activity and stability of peptide contained in DC7-2 is italicized.

| | | Cell signaling | | | Stability |
|---|---|---|---|---|---|
| | | activity | | Stability | in |
| Peptide | | (EC50, nM) | | in serum | intestine |
| ID | Sequence | Mean | STD | (t½, min) | (t½, min) |
| 36015 | Y PYIL (SEQ ID NO: 988) | 1.40E−07 | 1.01E−08 | 0.0091 | 2.0 |
| 36014 | W PYIL (SEQ ID NO: 989) | 1.33E−07 | 1.32E−08 | 0.0066 | 0.2 |
| 36013 | V PYIL (SEQ ID NO: 990) | 3.78E−08 | 2.71E−09 | 0.0094 | 0.3 |
| 36012 | T PYIL (SEQ ID NO: 991) | 4.36E−08 | 3.13E−09 | 0.0163 | 2.9 |
| 36011 | S PYIL (SEQ ID NO: 992) | 2.25E−08 | 1.62E−09 | 0.0241 | 0.0 |
| 36010 | R PYIL (SEQ ID NO: 40) | 1.18E−09 | 1.00E−10 | 0.0176 | 1.1 |
| 36009 | Q PYIL (SEQ ID NO: 993) | 2.05E−08 | 1.47E−09 | 0.0372 | 0.2 |
| 36008 | P PYIL (SEQ ID NO: 994) | 9.61E−09 | 6.98E−10 | 0.0197 | 2.6 |
| 36007 | N PYIL (SEQ ID NO: 995) | 3.93E−08 | 2.81E−09 | 0.0148 | 0.4 |
| 36006 | M PYIL (SEQ ID NO: 996) | 1.62E−08 | 1.17E−09 | 0.0034 | 0.2 |
| 36005 | L PYIL (SEQ ID NO: 997) | 4.10E−08 | 4.52E−09 | 0.0373 | 0.1 |
| 36004 | K PYIL (SEQ ID NO: 9) | 7.71E−09 | 8.77E−10 | 0.0124 | 0.2 |
| 36003 | 1 PYIL (SEQ ID NO: 998) | 2.38E−08 | 2.63E−09 | 0.0132 | 0.2 |
| 36002 | H PYIL (SEQ ID NO: 999) | 3.56E−08 | 3.94E−09 | 0.0366 | 0.1 |
| 36001 | G PYIL (SEQ ID NO: 1000) | 1.74E−08 | 1.94E−09 | 0.0125 | 0.3 |
| 36000 | F PYIL (SEQ ID NO: 1001) | 1.95E−08 | 2.17E−09 | 0.0343 | 2.4 |
| 35999 | E PYIL (SEQ ID NO: 1002) | 1.02E−07 | 1.12E−08 | 0.0405 | 3.6 |
| 35998 | D PYIL (SEQ ID NO: 1003) | 1.58E−07 | 1.74E−08 | 0.0352 | 12.5 |
| 35997 | C PYIL (SEQ ID NO: 1004) | 6.99E−08 | 7.73E−09 | 0.0349 | 0.2 |
| 35996 | A PYIL (SEQ ID NO: 1005) | 1.18E−08 | 1.31E−09 | 0.0036 | 0.4 |

In conclusion, the results demonstrates that octa- and heptapeptides are more stable, and that the N-terminal aa in the hexapeptide has a significant implication on the stability.

As compared to the hexapeptide of a natural hormone, neurotensin (8-13) (NT with the sequence RRPYIL), one specific peptide of the present invention DKPYIL is nearly 100 times more stable in serum and around 100-1000× more stable in intestine homogenate.

In Vivo Studies

Acute effects of DC7-2 on satiety is shown in FIG. 12, 17-20.

### Example 3

Although DC7-2 is 100-1000 times more stable than the active fragment of neurotensin in a rodent intestine model of degradation, it is still degraded by a combination of intestine homogenate and pancreatin in vitro.

Protection of DC7-2 could be provided by a combination of protease inhibitors (as a potato protein extract) and crude protein hydrolysate (from bovine heart), neither of which alone were effective for prevention of degradation.

Protease activity in the small intestine and pancreas was modelled in vitro by combining a homogenate of mouse intestine (5 mg of protein per ml) and pancreatin (6.7 mg of protein per ml, Sigma P7545 from porcine intestine) and incubating with DC7-2 in absence or presence of i) potato protein isolate (PP) obtained by filtration from commercial production of starch and ii) crude protein hydrolysates. Whereas PP alone could provide partial protection, whereas crude protein hydrolysate alone provided little or no protection, the combination of the two was able to fully protect DC7-2 in the in vitro model. (FIG. 21).

Swiss-Webster mice (25-35 g bodyweight) were fasted 3-6 h and then gavaged with 1.0 ml of PP (potato protein 100 mg/ml), DC7 crude protein hydrolysate from bovine heart (400 mg/ml), DC7-2 (10 mg/ml), or the combination. All proteins/peptides were dissolved in water and adjusted to pH 7.0. Mice were gavaged again after 20 min with 0.25 ml of 40% (w/w) poly(ethylene glycol), MW 8 kDa containing 0.2% phenol red. Mice were sacrificed after another 20 min and stomachs homogenized in water for measurement of phenol red by absorbance after adjusting to alkaline pH with NaOH. Data are mean+SEM. Number of mice per group were 9 (PP), 10 (DC7), 23 (DC7-2), 11 (PP+DC7+DC7-2). (FIG. 22).

### Example 4

In Vitro Protection Assay

SIGMAFAST™ Protease Inhibitor Cocktail tablet (S8830, Sigma-Aldrich, Denmark), trypsin inhibitor (P9253, Sigma-Aldrich, Denmark), DPP IV inhibitor Sitagliptin (Januvia®, Merck Sharp and Dohme Ltd, Great Britain), carboxypeptidase inhibitor (CO279, Sigma-Aldrich, Denmark), ACE inhibitor captopril (C4042, Sigma-Aldrich, Denmark), potato protein and protein extract (KMC A.m.b.a, Denmark) were dissolved in deionized water and diluted in HBSS containing 100 mM HEPES (pH 7.4). DC7-2 was mixed with either inhibitors or 10% (w/v) potato protein products or 10% (w/v) potato protein in 40% (w/v) hydrolyzed bovine whey protein (Lacprodan DI-3065, Arla Food Ingredients, Denmark) or buffer and incubated with 0.16 g/L intestine homogenate at 37° C. for 30 min. Enzymes were inactivated by adding 1 M phosphoric acid to a final concentration of 0.4 M. Effect of inhibitors were determined by comparison to controls for zero degradation, with addition of 1 M phosphoric acid to 0.4 M prior to addition of intestine homogenate. All digests were neutralized to pH 7.2-7.4 by NaOH/HEPES/HBSS buffer before performing the calcium response assay as described above.

In Vivo Protection Assay

Swiss Webster mice (Janvier Laboratories, France) were housed in a reverse 12-hour light-dark cycle [12:00 p.m-12:00 a.m, light period] and had free access to feed and water. Before the experiment, mice were fasted for 3 hours without access to water. 500 μl of 10 mg/ml DC7-2 (Casio ApS, Denmark) mixed with 10% (w/v) potato protein (KMC A.m.b.a, Denmark) and 40% hydrolyzed bovine heart protein (DC7, Danish Crown Ingredients, Denmark) was orally administrated. After 50 minutes, mice were terminated by cervical dislocation and stomach and intestine divided into 3 equal sized pieces were immediately removed and homogenized in 5 ml 0.4 M phosphoric acid to deactivate enzymes and stop degradation of DC7-2. Samples were centrifuged at 14.500 rpm for 5 minutes followed by neutralization to pH 7.2-7.4 with NaOH/HEPES/HBSS and then immediately used for determination of DC7-2 activity. Recovered activity was compared to the amount of DC7-2 in the gavage mixture.

Gastric Emptying Studies in Mice

Gastric emptying was assessed in male Swiss Webster mice (Janvier Laboratories, France) after gavage with 250 μl of 0.2% phenol red (MERCK, 7241), which is not absorbed from the stomach but disappears only by emptying into the small intestine, with the slight modification of using 40% polyethylene glycol (PEG, P2139, Sigma-Aldrich, Denmark) as thickener. At t=−20 min, mice were gavaged with 500 ul of DC7-2 dissolved in water, in 10% potato protein, in 40% DC7 or a combination hereof or with protein solutions alone as control. At t=0 min, mice were gavaged with 250 μl of 0.2% phenol red in PEG. Mice were sacrificed 20 min after oral administration of phenol red solution. Stomachs were dissected and homogenized in 5 ml of deionized water, centrifuged at 10.000×g at room temperature for 10 minutes, and 100 μl supernatant was mixed with 100 μl of 1M NaOH before absorbance was measured at 560 nm. Gastric content of phenol red was calculated as percent of total gavage load (kinetic experiment) or after subtraction of vehicle alone for dose-response curves.

Feed Intake Studies in Mice

Female Swiss Webster mice (Janvier Laboratories, France) were fed a standard diet with ad libitum access to feed and water. The experiments were performed in a temperature-controlled room maintained at 23±2° C. with a reverse 12-hour light-dark cycle [11:00 p.m-11:00 a.m, light period]. Mice were single-housed for 1-day acclimation period with free access to feed and water. Animals were randomly assigned to three groups (n=24 per group) and fasted for 6 hours before beginning of experiments. Just before the beginning of the dark period, mice were gavaged with either water, vehicle alone (10% potato protein mixed with 40% DC7) or 5 mg DC7-2 in vehicle. After oral administration, the diet was made available and food consumption was measured every hour for the subsequent 6 hours. Data was plotted as accumulative feed intake (g/mouse).

Results:

Enzymatic Activities in Intestine Homogenate and Pancreatin

Enzyme specific substrates and inhibitors were applied to detect the presence of particular gastrointestinal and pancreatic proteases and peptidases in our intestinal homogenate preparation and a solution of commercially available porcine pancreatin. As expected, dipeptidyl peptidase IV (DPP IV) (FIG. 23A) activity was observed in intestine homogenate. Sitagliptin, a DPP IV inhibitor dose-dependently inhibited DPP IV activity in nanomolar range (EC50˜20 μg/L) while a mixture of protease inhibitors with broad specificity for inhibition of serine, cysteine and metalloproteases only exhibited dose-dependent DPP IV inhibitory effects at high concentrations (FIG. 23A). Intestinal and pancreatic trypsin activity was inhibited in a dose-dependent manner by two specific trypsin inhibitors from soybean and chicken egg white, respectively (FIG. 23B). The rate of substrate conversion in pancreatin was significantly higher than in intestine homogenate, reflecting different amounts of trypsin in the two preparations (FIG. 23B).

Besides trypsin, intestinal homogenate and pancreatin also contained chymotrypsin activity, although the amount of chymotrypsin was relatively low in intestine homogenate (FIG. 23C). A solution of KMC Potato Protein purified from potato tuber dose-dependently suppressed chymotrypsin activity, whereas bovine heart hydrolysate had no effect (FIG. 23C). Potato tubers accumulate chymotrypsin inhibitors, carboxypeptidase inhibitors, and Potato Kunitz inhibitor-1, a potent inhibitor of the animal pancreatic proteinase trypsin. KMC Potato Protein contains acid precipitated proteins isolated from potato juice during potato starch production, whereas KMC Potato Extract is the supernatant from the acid precipitation.

Pancreatin, but not intestine homogenate, contained detectable levels of carboxypeptidase activity, which was efficiently suppressed by a commercial carboxypeptidase inhibitor (CPI) from Potato tuber (FIG. 23D). Furthermore, solutions of KMC Potato Protein (PP), KMC Potato Extract (PE) and an up-concentrated ethanol extracted version hereof (PEEE) could also inhibit carboxypeptidase activity, although with lower potency than CPI (FIG. 23D).

Protection of DC7-2 from Degradation

To increase DC7-2 stability in biological matrices containing peptidases without employing strategies of targeted delivery methods or peptide modifications, we examined the effect of specific enzyme inhibitors and high concentrations of unspecific peptides on DC7-2 stability in in vitro degradation assays using a combination of intestine homogenate and pancreatin. Although DC7-2 was resistant to trypsin, we included trypsin inhibitors since trypsin may act indirectly on DC7-2 stability by its major role in the activation of proteolytic proenzymes such as chymotrypsin, elastase, and carboxypeptidase A in the duodenum.

A commercially available broad range protease inhibitor cocktail designed to inhibit serine, cysteine, aspartic and metalloproteases had partial protective effect on DC7-2 (FIG. 24A). A partial effect was also observed for the DPP IV inhibitor, sitagliptin, supporting our speculations of DC7-2 being DPP IV sensitive (FIG. 24A). Only small protective effects were observed with a specific trypsin inhibitor in correlation with DC7-2 being resistant to trypsin, but also indicating that trypsin in our preparation of intestine and pancreatin homogenate already had activated downstream effector proteases (FIG. 24A). The ACE inhibitor, captopril, did not protect DC7-2, indicating that ACE is not involved in DC7-2 degradation (FIG. 24A), although previous studies have implicated ACE in NT degradation. The best protection was obtained by carboxypeptidase inhibitors, highlighting the importance of L8 for DC7-2 activity (FIG. 24A).

Since KMC Potato Protein had inhibitory effects on chymotrypsin and carboxypeptidase activities, two different KMC Potato products were investigated for protective effects. Both potato products exhibited partial protection of DC7-2 from degradation by gastrointestinal and pancreatic peptidases (FIG. 24B).

Next, we investigated if better protection of DC7-2 could be obtained by including unspecific peptides from protein hydrolysates in order to keep proteases and peptidases not inhibited by potato protein busy digesting other peptides than DC7-2, thereby indirectly protecting DC7-2. Whey hydrolysate alone could not provide any protection of DC7-2, whereas KMC Potato Protein partially suppressed degradation as observed before (FIG. 24C). Interestingly, no degradation and hence complete protection of DC7-2 was obtained by a combination of 40% hydrolyzed whey protein with 10% KMC Potato Protein (FIG. 24C). Other protein hydrolysates had the same effect as whey protein hydrolysate in combination with potato proteins.

Oral Administration of DC7-2 Reduces Gastric Emptying and Feed Intake in Mice

Since we previously have shown that intraperitoneal administration of DC7-2 reduces feed intake and delays gastric emptying in mice, we tested, first, if DC7-2 had effects on gastric emptying after oral administration. Mice were administered with DC7-2 with or without protection by a 2-step gavage method introducing DC7-2 at 20 minutes prior to a gavage mixture containing phenol red to assess gastric emptying. Gastric emptying was dramatically slower in mice treated with DC7-2 protected by KMC Potato Protein and irrelevant peptides from bovine heart hydrolysate (FIG. 25A). KMC Potato Protein alone had no effect, whereas DC7 alone reduced gastric emptying by a factor of two and the combination of DC7 and KMC Potato Protein resulted in a further decrease corresponding to an additive effect of the two components (FIG. 25A), which we regard as a general protein effect on gastric emptying. A small but significant effect on gastric emptying was observed with DC7-2 without protection compared to water-gavaged controls (FIG. 25A, P=0.000023). An intermediate delay in gastric emptying was observed when combining DC7-2 with either KMC Potato Protein or DC7, supporting our finding of partial protection using just one of these two components (FIG. 25A, FIG. 24C). A dose-dependent relationship between phenol red in the stomachs and the dose of DC7-2 protected in DC7 and KMC Potato Protein with a calculated EC50 value of 20 mg per kg body weight was obtained (FIG. 25B). In contrast, the EC50 value of intraperitoneal administration of DC7-2 was 3.3 mg per kg bodyweight, indicating that DC7-2 was not fully protected from degradation in the complex biological matrix of an in vivo setting.

Secondly, it was tested if oral administration of DC7-2 had any effects on feed intake. Groups of mice received DC7-2 in vehicle (KMC Potato Protein+DC7), vehicle alone for comparison or water by gavage and feed intake was measured every hour during the subsequent 6 hours. As observed with intraperitoneal administration, the rate of feed intake was only temporarily reduced by DC7-2. After approximately 2 hours, the effect was reverted, although the overall feed intake remained significantly decreased at all time points after oral administration of 5 mg DC7-2, documenting that mice did not compensate for the lower amounts of feed consumed initially. Vehicle alone also resulted in a temporarily reduction in feed intake, although not to the same extent as in combination with DC7-2, showing that protein in general affects feed intake supporting our results from gastric emptying since a correlation between gastric emptying and feed intake have been shown in previous studies (FIG. 25C).

Finally, the in vivo stability of DC7-2 mixed with KMC Potato Protein and DC7 was determined. At 50 minutes after oral administration of 5 mg of DC7-2, mice were terminated by cervical dislocation and activity of DC7-2 in stomach and intestine were determined by calcium response. Compared to the amount of DC7-2 in the gavage mixture, only 9% DC7-2 was recovered from the gastrointestinal tract, distributed with 6% in the stomach, 0.69% in the first part of the intestine, 1.35% in the middle part and 0.66% in the last part (FIG. 25D). Using the same amounts of DC7-2 and components for protection, a significant delay in gastric emptying and reduced feed intake was observed (FIGS. 25A and C), suggesting that an even better in vivo effect after oral administrated can be achieved if protection of DC7-2 is further optimized.

In our study, specific carboxypeptidase inhibitors offered the best protection in agreement with DC7-2 being carboxypeptidase sensitive and the C-terminal L8 being important for activity. The DPP IV inhibitor, sitagliptin, provided partial protection, so did the SIGMAFAST inhibitor cocktail with broad protease specificity, documenting that DC7-2 was sensitive to DPP IV, but also to other proteases. Irrelevant peptides from hydrolysates were used as substrates for enzymatic hydrolysis by unknown proteases. A combination of irrelevant protein with a solution of potato protein containing chymotrypsin and carboxypeptidase inhibitors resulted in an almost complete protection of DC7-2. The protective effect was shown to be independent of the sources of hydrolysate in our ex vivo assay.

### Example 5

C-Terminal Modifications and Extensions.

Removal of C-terminal leucine resulted in almost complete loss of receptor activation as shown above. Modifying the C-terminus by amidation of the carboxylate, which is commonly used for stabilization of the C-terminus, also resulted in nearly completely loss of agonist activity with a shift in EC50 of more than three orders of magnitude (FIG. 26a).

We then decided to extend the C-terminus with one or two of the amino acids that occur naturally in the a-actinin-2 sequence (FIG. 26b). Consistent with the inactivity of the amide, extension with A resulted in an ˜1200-fold loss of potency compared to DC7-2. Surprisingly, extension with two amino acids (AE) almost restored activity with only a 10-fold loss in potency compared to DC7-2.

Since all modifications of the DC7-2 structure thus far have pointed to great flexibility at the N-terminus, we had a biotinylated version prepared for binding studies. However, N-terminal biotinylation reduced potency 10-fold (FIG. 26a). We also did swaps of D and S in the heptapeptide or replaced D with S in the hexapeptide (FIG. 26c). Whereas the former had no consequences for potency (8 vs 10 nM for the two heptapeptides), replacing D with S in the hexapeptide resulted in three-fold improvement of activity.

1.-33. (canceled)

34. A composition comprising i) a polypeptide comprising the amino acid sequence
(formula III, SEQ ID NO: 3)
AA1-AA2-AA3-AA4-AA5-AA6-AA7-AA8,
wherein AA1 is an optional amino acid selected from A, L, I, and V; AA2 is an optional amino acid selected from S, T, G, A, N, E and D; AA3 is an optional amino acid selected from D, E, and G; AA4 is K; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; and ii) a protease inhibitor, wherein the composition preferably is an oral dosage form.

35. The composition according to claim 34, wherein the polypeptide comprises the amino acid sequence
(formula I, SEQ ID NO: 1)
AA1-AA2-AA3-AA4-AA5-AA6-AA7-AA8-E*
or
(formula II, SEQ ID NO: 2)
R1-AA1-AA2-AA3-AA4-AA5-AA6-AA7-AA8-E*-R2
wherein AA1 is an optional amino acid selected from A, L, I, and V; AA2 is an optional amino acid selected from S, T, G, A, N, E and D; AA3 is an optional amino acid selected from D, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; R1 defines the N-term (—NH2) or a protection group; wherein E* is C-terminal extension with 1-10 of any amino acids, and preferably wherein E* is 1-10 amino acids of the naturally occurring amino acid sequence of α-actinin-2, and in particular wherein E* is selected from A, AE, AEE, AEEL (SEQ ID NO:1016), AEELR (SEQ ID NO:1017), AEELRR (SEQ ID NO:1018), AEELRRE (SEQ ID NO:1019), AEELRREL (SEQ ID NO:1020), AEELRRELP (SEQ ID NO:1021), and AEELRRELPP (SEQ ID NO:1022); R2 defines the C-term (—COOH).

36. (canceled)

37. The composition according to claim 34, wherein the proteinase inhibitor is pI2, which is a potato protein extract present in said composition in an amount higher than 2%, 3%, 4%, 5%, 7%, 8%, 9%, 10%, 15%, 20%, 25%, 30%, 35%, 40%, 45%, or 50% of total protein, preferably a potato protein extract with a content of trypsin and chymotrypsin inhibitors higher than 10% of total protein, such as higher than 20%, 25%, 30%, 35%, 40%, 45%, or 50% of total protein of said potato protein extract and/or a potato protein extract with a content of carboxypeptidase inhibitors higher than 1%, such 2%, 3%, 4%, 5%, 6%, 7%, 8%, 9%, 10%, 12%, 14%, 16%, 18%, 20%, 25%, 30%, or 35% of total protein of said potato protein extract.

38.-47. (canceled)

48. The composition according to claim 34, wherein the composition is a food composition or a fermented composition or a dairy product or a pharmaceutical composition or a nutritional composition.

49.-53. (canceled)

54. The composition according to claim 34, which is an oral dosage form selected from the group comprising tablets, capsules, caplets, slurries, sachets, suspensions, chewing gum, and powder formulation that may be dissolved in a liquid, such as water, milk, juice, or yogurt.

55.-57. (canceled)

58. The composition according to claim 34, which composition further comprises a protein substrate for any one protease, such as an endoproteinase, such as trypsin or chymotrypsin, and an exoproteinase, such as carboxypeptidases, such as Carboxypeptidase A and B.

59. (canceled)

60. The composition according to claim 58, which protein substrate is derived from an animal, such as from meat, such as bovine heart, such as from blood or plasma, or from a milk product, such as whey, from a plant, such as from cereals, such as wheat, legumes, soy, rice, nuts, seeds, from micro or macroalgae, such as Spirulina, or from a microorganism, such as bacteria, such as Bacillus sp., or from fungi, such as yeast, such as Saccharomyces sp.

61.-63. (canceled)

64. A method of promoting satiety or for reducing feed intake or for preventing or reducing the incidence of obesity or for reducing or treating cardiovascular diseases, atherosclerosis, hypertension, hepatosteatosis, cancer and/or diabetes in a subject in need thereof, comprising enteral administering to the subject in need thereof the composition as defined in claim 34.

65.-66. (canceled)

67. The methods according to claim 64, wherein said polypeptide does not comprise or consist of any one of the sequences
(SEQ ID NO: 5)
AVTEKKYILYDFSVTS,
(SEQ ID NO: 38)
PRRPYIL,
(SEQ ID NO: 39)
RRPYIL,
(SEQ ID NO: 40)
RPYIL,
(SEQ ID NO: 41)
RRPWIL,
(SEQ ID NO: 42)
KRPYIL,
(SEQ ID NO: 43)
KKPYIL,
(SEQ ID NO: 9)
Adamantoyl-KPYIL,
(SEQ ID NO: 44)
H-Lys-psi(CH₂NH)Lys-Pro-Tyr-Ile-Leu-OH

68.-73. (canceled)

74. The composition according to claim 34, wherein the proteinase inhibitor is proteinase inhibitor II (PI2).

75. The composition according to claim 34, wherein the proteinase inhibitor is proteinase inhibitor II (PI2) derived from a potato protein extract.

76. The composition according to claim 34, wherein AA1 is A and/or wherein AA2 is S and/or wherein AA3 is D and/or wherein AA5 is P and/or wherein AA6 is Y and/or wherein AA7 is I and/or wherein AA8 is L.

77. The composition according to claim 34, wherein the polypeptide is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

78. The composition according to claim 34, wherein the polypeptide comprises an amino acid sequence selected from the group consisting of ASDKPYILA (SEQ ID NO:1006), ASDKPYILAE (SEQ ID NO:1007), ASDKPYILAEE (SEQ ID NO:1008), ASDKPYILAEEL (SEQ ID NO:1009), ASDKPYILAEELR (SEQ ID NO:1010), ASDKPYILAEELRR (SEQ ID NO:1011), ASDKPYILAEELRRE (SEQ ID NO:1012), ASDKPYILAEELRREL (SEQ NO:1013), ASDKPYILAEELRRELP (SEQ 1D NO:1014), and ASDKPYILAEELRRELPP (SEQ ID NO:1015).
